(19) United States
(12) Patent Application Publication
(10) Pub. No.: US 2007/0059699 A1
(43) Pub. Date: Mar. 15, 2007

(54) Methods for detecting toxic and non-toxic cyanobacteria

(76) Inventors: Kaarina Sivonen, Helsinki (FI); Anne Rantala, Helsinki (FI); Leo Rouhianinen, Helsinki (FI); David Fewer, Helsinki (FI); Pirjo Rajaniemi, Helsinki (FI); Annick Wilmotte, Tiliff (BE); Christophe Boutte, Le Charnbon Feugerolles (FR); Stana Grubisic, Liege (BE); Pierre Balthasart, Liege (BE); Gianluca De Bellis, Segrate Milano (IT); Ermanno Rizzi, Segrate Milano (IT); Andrea Frosini, Segrate Milano (IT); Bianca Castiglioni, Segrate Milano (IT); Stefano Ventura, Bologna Bo (IT); Maria Angela Mugnai, Prato Po (IT)

Correspondence Address: John Dodds, 1707 N St N W, Washington, DC, 20036, US

(21) Appl. No.: 10/557,426

(22) PCT Filed: May 21, 2004
(86) PCT No.: PCT/FI04/00310

§ 371 Date: Nov. 6, 2006

(30) Foreign Application Priority Data
May 21, 2003 (FI) 20030771

(51) Int. Cl. C12Q 1/68; C12P 21/06; C07H 21/04; C12N 1/21; C07K 14/195

(52) U.S. Cl. 435/6; 435/69.1; 435/320.1; 435/252.3; 530/350; 536/23.7

(57) ABSTRACT

This invention is related to a method for detecting toxic and non-toxic cyanobacteria. The method comprises that nucleic acid from a biological sample is brought into contact with an oligonucleotide designed to be specific for the mcy gene, in particular mcyE and/or mcyD, and with an oligonucleotide designed to be specific for 16SrDNA, and the presence or absence of toxic cyanobacteria is detected by a suitable molecular biology method. The invention is related also to oligonucleotides used in the method.

This invention relates to a method for detecting toxic and non-toxic cyanobacteria. This invention relates also to oligonucleotides, which can be used in the detection method.

### BACKGROUND OF THE INVENTION

Cyanobacteria produce a wide variety of bioactive compounds. Many of these are potent toxins, which cause health problems for animals and humans when producer organisms occur in masses in lakes and water reservoirs (Sivonen and Jones, 1999). Most well known of the cyanobacterial toxins are the hepatotoxic heptapeptides, microcystins. The general structure of microcystins is cyclo(-D-Ala-X-D-MeAsp-Z-Adda-D-Glu-Mdha-), where X and Z are variable L-amino acids, D-MeAsp is D-erythro-β-methylaspartic acid, Mdha is N-methyldehydroalanine and Adda is 3-amino-9-methoxy-2,6,8-trimethyl-10-phenyldeca-4,6-dienoic acid. More than 65 structurally different microcystins are known (Sivonen and Jones, 1999). Most common variants have L-leucine and L-arginine in the positions of X and Z, respectively, and demethylated forms are also frequently found. Toxicity of microcystins is caused by the inhibition of protein phosphatases 1 and 2A (MacKintosh et al., 1990). The level of inhibition varies depending on the structure, but the Adda and D-Glu moieties, which are almost invariable in microcystins, are essential for the inhibition (Goldberg et al., 1995) and hence for the toxicity.

Microcystins have been found predominantly in cyanobacteria of three platonic, bloom-forming genera, Anabaena, Microcystis and Planktothrix (Sivonen and Jones, 1999). Not all members of these genera make microcystins and both toxic and non-toxic strains occur in the same species. Toxic and non-toxic strains of Anabaena, Microcystis or Planktothrix cannot be separated based on the classical morphological taxonomy or ribosomal gene sequencing (Lyra et al., 2001). On the other hand, one stain may produce different microcystins and also other peptides simultaneously (Sivonen et al., 1992; Fujii et al., 1996; Fastner et al., 2001.

Peptide synthetase genes were shown to be required for the synthesis of microcystins (Dittmann et al., 1997). Recently, the gene clusters encoding microcystin synthetase were sequenced and characterized from the unicellular Microcystis aeruginosa (Nishizawa et al., 2000; Tillet et al., 2000) and from the filamentous Planktothrix agardhii (Christiansen et al., 2003). It was demonstrated that the microcystins biosynthesis is a combination of peptide and polyketide synthesis (Nishizawa et al., 2000; Tillet et al., 2000).

The microcystin synthetase gene region spans about 55 kb, and includes genes for peptide synthetases (mcyA, -B, -C), polyketide synthases (mcyD), mixed peptide synthetase and polyketide synthases (mcyE, -G), and tailoring enzymes Tirett. et al. (2000), Nishizawa et al. (2000).

Microcystin producers among the filamentous, nitrogen-fixing genus, Anabaena, are found in North America, in France and in Northern Europe, where they frequently develop massive growth in lakes and reservoirs (Sivonen and Jones, 1999). The bioactive peptides produced by Anabaena 90 have been characterized: three microcystins (MCYST-LR, MCYST-RR and D-Asp-MCYST-LR; Sivonen et al., 1992), two seven-residue depsipeptides (anabaenopeptilide 90A and 90B), and three six-residue peptides having an ureido linkage (anabaenopeptins A, B and C; Fujii et al., 1996). However, the microcystin synthetase gene region from Anabaena has not been sequenced.

Based on the sequence data available, various DNA probes and primers have been designed and used to discriminate between toxic microcystin-producing and non-toxic non-microcystin producing genotypes by hybridization and PCR However, the existing primers deduced from Microcystis mcy genes, reliably identify potential microcystin-producers only in Microcystis and fail to amplify mcy sequences from part of microcystin containing strains of other genera. There is therefore a great need for oligonucleotides, which could be used as probes and primers in detecting toxic cyanobacteria also in genera other than Microcystis. Such oligonucleotides should discriminate between toxic microcystin-producing and non-toxic non-microcystin producing genotypes in various molecular biology methods, such oligonucleotides should be specific to the studied cyanobacteria genera and the oligonucleotides should be able to discriminate the most important or dominating microcystin producing cyanobacteria genera from one another.

It would be also of advantage if non-toxic cyanobacteria could be identified.

### SUMMARY

It is the aim of the present invention to eliminate the problems associated with the prior art.

One object of this invention is to provide a method for the detection of toxic cyanobacteria.

In this invention it has been surprisingly found that by designing oligonucleotides to be specific for mcyE gene of the microcystin synthetase gene region, it is possible to detect cyanobacteria from all of the most potent toxin producing cyanobacteria genera. In addition it is possible to identify which cyanobacterial genus produces the toxin.

In particular, the oligonucleotides are designed to be specific for a region of mcyE gene responsible for adding Adda and D-glutamate to the immature synthesis product.

More specifically, the oligonucleotides are designed to be specific for a region of mcyE gene region catalyzing a peptide synthesis between Adda-D-glutamate and dehydroalanine and to the adenylating region. It is assumed that the step of adding Adda-D-glutamate-dipeptide is decisive for toxicity of the product. However, it is surprising that oligonucleotides designed to be specific for this region are genus specific and at the same time capable of identifying cyanobacteria from all other toxin-producing genera. Oligonucleotides of this invention can identify toxin producers at least among Anabaena, Microcystis, Planktothrix, Nostoc and Nodularia genera.

In this invention the whole microcystin synthetase gene region from Anabaena was sequenced. Before this invention it had not been possible to compare the sequences of microcystin synthetase gene region from the main microcystin-producing cyanobacteria genera.

The oligonucleotides of this invention can be used in detecting toxin-producing cyanobacteria by using various molecular biology methods. Such methods are for example hybridization, PCR, reverse transcriptase PCR, QRT-PCR, LCR, LDR and minisequencing.

These methods can be combined with a microarray method. In a preferred detection method ligase detection reaction (LDR) is used together with a microarray method. Another preferred detection method is quantitative PCR (QRT-PCR).

Furthermore, the oligonucleotides of this invention can be used in detecting toxin-producing cyanobacteria together with a detection method using oligonucleotides designed to be specific for any other mcy gene, such as mcyA or mcyD gene.

One highly preferred embodiment of this invention is the use of the oligonucleotides of this invention together with oligonucleotides designed to be specific for 16S rRNA gene. Cyanobacterial genera can be identified based on the 16S rRNA gene. When oligonucleotides designed to be specific for mcyE (or some other mcy gene, such as mcyD) and for 16S rRNA gene are used together for example in the microarray method, it is possible to detect and identify both toxin- and non-toxin-producing genera. It is of great advantage that the oligonucleotides designed to be specific for mcyE and for 16S rRNA gene can be used under the same conditions. The LDR can be carried out under the same conditions and the hybridization in microarray on the same slide. This makes the monitoring of non-toxin cyanobacteria- and toxin-producing cyanobacteria technically easy and much more useful.

The detection method of the present invention can also be combined with a detection method measuring microcystin concentration, cell number, cell density or biomass. For example, mcyE copy number can be determined together with microcystin concentration and cell density and the main putative microcystin producers can be indicated.

One object of this invention are fragments of mcyE gene which are responsible for adding Adda and D-glutamate to the immature synthesis product in microcystin synthesis. In particular, the fragments are responsible for adding Adda-D-glutate dipeptide to dehydroalanine. Such fragments are or are located in the sequences selected from the group comprising SEQ ID NO. 1 to SEQ ID NO: 34 as shown in FIG. 19 A to H or comprising sequences SEQ ID NO: 35 to SEQ ID NO: 39 as shown in FIG. 15 A to C.

One object of this invention are furthermore oligonucleotides designed to be specific for any of the above mentioned fragments of mcyE gene, in particular for sequences selected from the group comprising SEQ ID NO. 1 to SEQ ID NO: 34 as shown in FIG. 19 A to H or sequences SEQ ID NO: 35 to SEQ ID NO: 39 as shown in FIG. 15 A to C or for fragments of said sequences.

Preferred oligonucleotides are primers mcyE-F2 (SEQ ID Nos: 64), AnamcyE-12R (SEQ ID NO: 65) and MicmcyE-R8 (SEQ ID NO:66) which can be used for example in amplifying target (or sample) nucleic acid by PCR.

Preferred oligonucleotides are discriminating probes of SEQ ID NO: 40 to SEQ ID NO: 45 and common probes of SEQ ID NO: 46 to SEQ ID NO: 51, which can be used for example in the ligase detection reaction.

One object of this invention is furthermore the mcyE gene from the Anabaena genus encoding the amino acid sequence of SEQ ID NO: 67 or a sequence having at least 80% identity, preferably 90%, more preferably 95% identity to said sequence, or a fragment of said sequence having polymorphic sites which make possible of designing oligonucleotides to be specific for the fragment.

One further object of this invention is mcyE gene from Anabaena genus having the nucleic acid sequence SEQ ID NO: 68 or a sequence having at least 80% identity, preferably 90%, more preferably 95% identity to said sequence, or a fragment of said sequence having polymorphic sites which make possible of designing oligonucleotides to be specific for the fragment.

One object of this invention is furthermore the mcyD gene from the Anabaena genus encoding the amino acid sequence of SEQ ID NO: 69 or a sequence having at least 80% identity, preferably 90%, more preferably 95% identity to said sequence, or a fragment of said sequence having polymorphic sites which make possible of designing oligonucleotides to be specific for the fragment.

One further object of this invention is mcyD gene from Anabaena genus having the nucleic acid sequence SEQ ID NO: 70 a sequence having at least 80% identity, preferably 90%, more preferably 95% identity to said sequence, or a fragment of said sequence having polymorphic sites which make possible of designing oligonucleotides to be specific for the fragment.

One object of this invention are fragments of mcyD gene. Such fragments are or are located in the sequences selected from the group comprising SEQ ID NO. 131 to SEQ ID NO: 149 as shown in FIG. 38 A to F.

One further object of this invention are oligonucleotides which can be used as discriminating probes and which are selected from the group comprising SEQ ID NO: 71 to SEQ ID NO: 90, and common probes which are selected from the group comprising SEQ ID NO: 91 to SEQ ID NO: 110. These primers and probes can be used for example in the ligation detection reaction.

Still further object of this invention is a kit for the detection of toxic cyanobacteria by the microarray method. The kit preferably comprises

discriminating and common probes designed to be specific for mcyE gene;

DNA or RNA zip and complementary zip codes assigned to be specific for selected cyanobacterial genera.

One still further object of this invention is a kit for detection of toxic cyanobacteria by hybridization. The kit preferably comprises

primers designed to be specific for the mcyE gene;

probes designed to be specific for selected cyanobacterial genera.

In the kit may be used alternatively or in addition probes and primers designed to be specific for mcyD gene or other mcy gene.

According to a highly preferred embodiment the kit comprises in addition to probes and primers designed to be specific for mcy gene (such as mcyE and/or mcyD) also probes and primers designed to be specific for 16 S rRNA gene.

Other features, aspects and advantages of the present invention will become apparent from the following description and appended claims.

### BRIEF DESCRIPTION OF THE FIGURES

FIG. 1. The microcystin synthetase gene cluster of Anabaena strain 90, biosynthetic model for the formation of microcystin-LR and the general structure of microcystins. The symbols for the domains are: A, adenylation; C, condensation; T, thiolation; NMT, N-methyltransferase; EP, epimerase; TE, thioesterase; KS, β-ketoacyl synthase; AT, acyltransferase; CM, C-methyltransferase; DH, dehydratase; KR, β-ketoacyl reductase; ACP, acyl carrier protein; AMT aminotransferase. Adda is 3-amino-9-methoxy-2,6,8-trimethyl-10-phenyldeca-4,6-dienoic acid, X and Z are variable amino acids. The arrows point to three methyl groups, which are putatively introduced by the C-methyltransferase domains. The way of cyclization of the microcystin precursor is shown with an arrow on the right of the picture.

FIG. 2. A. Comparison of the putative C-methyltransferase domains in McyG, McyD and McyE of Anabaena 90 with three bacterial C-methyltranferase domains in the region of the conserved motifs:

1. (VIL)(LV)(DE)(VI)G(GC)G(TP)G; 2. (PG)(QT)(FYA)DA(IVY(FI)(CVL) and 3. LL(RK)PGG(RIL)(LI)(LFIV)(IL) (Kagan and Clarke, 1994). EpoE is the polyketide synthase in epothilone biosynthesis of Sorangium cellulosum (AF217189), HMWP1 is the high-molecular-weight-protein in yersiniabactin biosynthesis coded by irp1 of Yersinia enterocolitica (Y12527) and ECUbiE is Escherichia coli C-methyltransferase, UbiE (P27851). Residues in bold letters (in the boxed areas) are identical to the consensus amino acids of the motifs. Amino acids (outside of the boxed areas), which are identical in at least five of the six sequences, are shaded.

B. Alignment of the aminotransferase domain of Anabaena 90 McyE, AmcyEamt, with other known aminotransferase domains and with two aminotransferases of Escherichia coli. McyEamt and PmcyEamt are from mcyE of Microcystis aeruginosa PCC7806 (AF183408) and of Planktothrix agardhii CYA126 (AJ441056), respectively. ItuAamt is from itrin synthetase of Bacillus subtilis RB14 (AB050629) and MycAamt from mycosubtilin synthetase of Bacillus subtilis ATCC6633 (AF184956). ECGSA is glutamate-1-semialdehyde aminotransferase (F90648) and EcArgD is ArgD, acetylornithine aminotransferase (P18335). The conserved pyridoxal-5′-phosphate-binding residues (Mehta et al., 1993), an aspartate and a lysine, are marked with the asterisks. Amino acids, which are the same in at least five of the seven proteins, are shaded.

FIG. 3. Motif sequence alignments of (A) dehydratase (DH) and (B) ketoreductase (KR) domains of Anabaena 90 microcystin synthetase, AMCD-DH2, AMCD-DH3, AMCG-KR1, AMCG-KR2 and AMCD-KR3, with rifamycin synthase, RifE-DH10 and RifE-KR10 (Amycolatopsis mediterranei; AF040570), and rapamycin synthase, RapA-DH4, RapB-DH10, RapA-KR4 and RapB-KR10, (Streptomyces hygroscopicus; X86780). The conserved residues of (A) the active site motif H(X)₃G(X)₄P (Aparicio et al., 1996) and of (B) the NAD cofactor binding site, GXGXX(G/A)(X)₃(G/A), (Scrutton et al., 1990) are marked with asterisks. Amino acids which are invariant in all proteins, are in bold letters (A) and (B). The numbers of the domains refer to the module of the particular synthase.

FIG. 4. Comparison of the motifs in acyltransferase (AT) domains of the microcystin synthetases with the consensus sequences of malonyl and methylmalonyl loading AT domains described by Ikeda et al. (1999). AT domains (AT1-AT4) are from Anabaena 90, AMcyG, AMcyD and AMcyE, from Microcystis aeruginosa, MMcyG, MMcyD and MmcyE (AF183408) and from Planktothrix agardhii, PMcyG, PMcyD and PmcyE (AJ441056). Bold letters indicate the amino acids, which are significantly specific to malonyl loading domains, and underlined, bold letters point out the residues, which are specific to methylmalonyl loading domains. Serines of the active site are marked with an asterisk.

FIG. 5. Alignments of the β-ketoacyl synthase (KS) (A) and acyl carrier protein (ACP) (1B), domains of Anabaena 90 microcystin synthetase with the KS and ACP domains of rapamycin synthase, RapA-KS1, RapA-ACP1 and RapC-ACP11 (Streptomyces hygroscopicus, X86780) and of rifamycin synthase, RifA-KS1 and RifA-ACP1 (Amycolatopsis mediterranei, AF040570) near the active sites. (A) AMCG-KS, AMCD-KS1, AMCD-KS2 and AMCEKS are from the KS domains of Anabaena 90 McyG, McyD and McyE, respectively. An asterisk marks the active site cysteines. The identical amino acids are in bold letters. The two histidine residues, which are invariant in PKS and fatty acid synthases (Aparicio et al., 1996) are underlined. (B) AMCG-ACP, ACD-ACP1, AMCD-ACP2 are from the ACP domains of Anabaena 90 McyG, McyD and McyE. The active site motif, which frequently is LG×DS, is underlined. The serine residues, which bind phospho-pantetheine, are indicated by an asterisk.

FIG. 6. The general structure of microcystins and nodularin. Microcystin is a cyclic peptide containing seven amino acids D-Ala-X-D-MeAsp-Z-Adda-D-Glu-Mdha, where X and Z represent variable L-amino acids, D-Me-Asp is D-erythro-β-methylaspartic acid, Mdha is N-methyldehydroalanine, and Adda is the β-amino acid, 3-amino-9-methoxy-2,6,8-trimethyl-10-phenyldeca-4,6-dienoic acid. Nodularin differs from microcystins by lacking the amino acids D-Ala and X, and having N-methyldehydrobutyrine Mdhb) in place of Mdha. The dashed line indicates the two amino acids absent in nodularins.

FIG. 7. Congruence between the 16S rRNA and rpoC1 data set and the microcystin synthetase gene data set. (A) A maximum-likelihood tree based on the 16S rRNA and rpoC1 data set (−1 nL-8004.26493). Branch lengths are proportional to sequence change. Maximum likelihood and maximum parsimony bootstrap values from 1000 bootstrap replicates are given above and below the line respectively. (B) A maximum-likelihood tree based on the mcyA, mcyD and mcyE data set (−1 nL=8781.50660). Branch lengths are proportional to sequence change. Maximum likelihood and maximum parsimony bootstrap values from 1000 bootstrap replicates are given above and below the line respectively.

FIG. 8. A maximum-likelihood tree based on the 16S rRNA gene showing the sporadic distribution of cyanobacterial genera known to produce microcystins. Strains of the genera Planktothrix, Microcystis , Anabaena and Nostoc produce microcystins while strains of the genus Nodularia produce nodularins. Toxic strains are indicated by bold font.

FIG. 9. Cycle treshold (Ct) values obtained by microcystin synthetase E (mcyE) quantitative real-time PCR (QRT-PCR) with external A) Anabaena standard strains of Anabaena 90 (O), Anabaena 315 (□), and Anabaena 202A1 (Δ) as well as with B) those of Microcystis strains Microcystis GL 260735 (O), Microcystis PCC 7806 (□), and Microcystis PCC 7941 (Δ) as a function of mcyE copy numbers. Error bars, which are almost hidden by the symbols, give the standard deviation for three independent amplifications.

FIG. 10. Microcystin concentration (x) (μg 1⁻¹) determined with ELISA and Anabaena as well as Microcystis microcystin synthetase E (mcyE) copy numbers (copies ml⁻¹) obtained with quantitative real-time PCR using Lake Tuusulanjärvi water samples collected during summer 1999. Gene mcyE copy numbers were calculated with the external standards of Anabaena 202A1 (▪), Anabaena 315 (□), Microcystis PCC 7806 (∘) and Microcystis PCC 7941 (●).

FIG. 11. Microcystin concentration (X) (μg 1⁻¹) determined with ELISA and Anabaena as well as Microcystis microcystin synthetase E (mcyE) copy numbers (copies ml⁻¹) obtained with quantitative real-time PCR using lake water samples collected from different water depths of four Lake Hiidenvesi basins on 15 Aug. 2001. Gene mcyE copy numbers were calculated with the external standards of Anabaena 202A1 (▪), Anabaena 315 (□), Microcystis PCC 7806 (∘) and Microcystis PCC 7941 (●).

FIG. 12. The cell numbers of the most dominant cyanobacterial genera in Lake Tuusulanjärvi in 1999 by light microscopy. The most dominant cyanobacterial genera were Anabaena (□), Microcystis (O) and Aphanizomenon (Δ).

FIG. 13. The cell numbers of the most dominant cyanobacterial genera in Lake Hiidenvesi on 15 Aug. 2001 by light microscopy. The most dominant cyanobacterial genera were Anabaena (□), Microcystis (O) and Aphanizomenon (Δ. The samples were taken from different water depths at the four basins of Lake Hiidenvesi.

FIG. 14. Clusters of group-specific mcyE gene consensus sequences.

FIG. 15. A; B, C. 800 bp consensus sequence of mcyE from Anabaena, Microcystis, Nodularia, Nostoc, Oscillatoria/Planktothrix (SEQ ID NOs 35 to 39).

FIG. 16. The principle of the DNA-chip (Microarray) method.

FIG. 17. Deposition scheme of the mcyE probes. Deposition scheme obtained using a non-contact dispensing system. Each zip code was spotted ten times. The deposition quality of the Zip Code oligonucleotides on the slides has been checked by means of hybridisations with Cy3 labelled poly(dT) complementary to the poly(dA)₁₀ sequence of each Zip Code.

FIG. 18. Hybridization results obtained using PCR amplified mcyE gene coming either from pure strains or from environmental samples as template in LDR.

FIG. 19 A-H. Alignment of 800 bp of nucleic acid sequences from 30 strains (+4 consensus sequences) from Anabaena, Microcystis, Nodularia, Nostoc, and Oscillatoria/Planktothrix genera (SEQ ID NOs 1 to 34).

FIG. 20. List of polymorhism positions, group-specific probes (discriminating probes SEQ ID NOs 40 to 45 and common probes 46 to 51) and their correspondent Zip Codes and complementary Zip Codes SEQ D NOs 52 to 57 and 58 to 63.

FIG. 21. Amino acid sequence encoded by Anabaena mcyE gene (SEQ ID NO 67).

FIG. 22 A-D. Nucleic acid sequence of Anabaena mcyE gene (SEQ ID NO 68).

FIG. 23 A, B. Amino acid sequence encoded by Anabaena mcyD gene (SEQ ID NO 69).

FIG. 24 A-D. Nucleic acid sequence of Anabaena mcyD gene (SEQ ID NO 70).

FIG. 25A. The cyanobacterial phylogenetic tree constructed using the NJ algorithm, according to a central database of processed sequences. ARB cyanobacterial 16S rRNA gene database we used contained 281 sequences from public databases and 59 from this study.

FIG. 25B. Updated ARB tree with Snowella sequences.

FIG. 25C. Updated ARB tree with subclustering of Anabaena and Aphanizomenon groups.

FIG. 26. Main features of LDR method coupled to a Universal Microarray.

Panel A: After the hybridization of a discriminating probe and a common probe to the target sequence (16s rRNA gene), ligation occurs only if there is perfect complementarity at the junction between the two probes. The reaction is thermally cycled.

Panel B: The LDR product is hybridized to an addressable Universal Microarray, where unique Zip code sequences have been spotted.

FIG. 27 A. Deposition scheme obtained using a contact dispensing system. Each Zip code was spotted four times, except universal Zip code (twelve times) and the Zip code corresponding to hybridization control (eight times). The deposition quality of the Zip Code oligonucleotides on the slides has been checked by means of hybridisations with Cy3 labelled poly(dT) complementary to the poly(da)₁₀ sequence of each Zip Code.

FIG. 27B. Deposition scheme of Universal Array for the detection of toxic and non-toxic cyanobacteria. The Universal Array is made of 8 subarray per slide. Each subarray is made of 208 spots including zipcodes for hybridization control, cyanobacterial universal probes, 16S rRNA gene specific probe, mcyE specific probe and empty spot as a negative control. Each specific zip code for the recognition of cyanobacteria universal probe, 16S RNA gene probe and mcyE gene probe is spotted in quadruplicate. The LDR positive control (zipcode no 63) is replicated 6 times, while the hybridization positive control (zipcode no 66) is replicated 8 times.

FIG. 28. Some results obtained using as LDR template PCR amplified 16S rRNA gene coming either from pure strains (both axenic and isolated in this study) or from cloned rDNA sequences.

Panel A: Aphanizomenon sp. 202; Panel B: Calothrix marchica Bai 71-96; Panel C: Leptolyngbya OBB19S12; Panel D: Lyngbya OBB32S04; Panel E: Microcystis 1BB 38S; Panel F: Nodularin sp. PCC73104/1; Panel G: Plankthotrix 1LT27S08; Panel H: Spirulina subsalsa PCC6313; Panel I: Synechococcus Heg 74-30; Panel J: Woronichinia OES46; Panel K: Cylindrospermum stagnale PCC7417; Panel L: Synechocystis PCC 6905; Panel M: Nostoc sp. 152; Panel N: Anabaena; Panel O: Cyanothece PCC 7418.

FIG. 29. Hybridization results obtained using LDR artificial mixes with unbalanced amounts of PCR products derived from the following cyanobacterial samples: Aphanizomenon sp. 202, Microcystis OBB 34S, Sprirulina subsalsa PCC6313, Calothrix sp. PCC7714, Woronichinia OES46 clone. Different ratios have been used: 100:1, 50:1, 100:5, 50:5, in which Aphanizomenon sp. 202 and Microcystis OBB 34S have been the more concentrated samples.

Panel A: Unbalanced 100:1 LDR mix, Panel B: 50:1 LDR mix; Panel C: 100:5 LDR mix; Panel D: 50:5 LDR mix; Panel E: unbalanced LDR mix performed with 500 fmol of the amplicon derived from Microcystis OBB 34S and 5 fmol of the PCR fragment obtained from Woronichinia OES46 clone.

FIG. 30A. Comparison of the results obtained using two LDR unbalanced mixes 100:1 (100 fmol of Microcystis OBB 34S and 1 fmol each of Spirulina, Woronichinia and Calothrix).

Panel A: The LDR unbalanced mix was prepared using 4U of Pfu DNA ligase.

Panel B: 8U of the enzyme was added-in the same LDR unbalanced mix described above.

FIG. 30B. 16S and mcyE detection onto universal Array. Example of quantification.

FIG. 31. Linear correlation between signal intensity and template concentration

FIG. 32. List of the group-specific 16S rRNA gene probes and their correspondent Complementary zip codes (SEQ ID NOs 111 to 130) (discriminating probes SEQ ID NOs 71 to 90, common probes SEQ ID NOs 91 to 110).

FIG. 33A, B. Cyanobacterial strains used to validate the LDR probes.

FIG. 34. Clones of 16S rRNA gene libraries obtained from environmental samples and used in the LDR reaction.

FIG. 35. PCR amplification from genomic DNA using 16S cyano primers and mcyE primers; primer F=mcyE-F2 and primer R=mcyE-R4; amplification protocol: 1×(3′, 95° C.), 30×(30″, 94° C.; 30″, 56° C.; 1′, 72° C.), 1×(10′, 72° C.).

FIG. 36. Ligation Detection Reaction for toxic and non-toxic cyanobacteria recognition.

FIG. 37. Hybridization on DNA chip.

FIG. 38 A to F mcyD sequence fragments from different cyanobacteria genera (SEQ ID Nos 131-149). In SEQ ED Nos 137, 138 and 139 N is T.

FIG. 39. List of the group-specific 16S rRNA gene probes (discriminating probes SEQ ID NOs 150 to 156) (common probes SEQ ID NOs 157 to 163) and C-zip Code sequences (SEQ ID Nos 164 to 170).

### DETAILED DESCRIPTION OF THE INVENTION

Definitions

By “nucleic acid from a biological sample” is in this invention meant any target or sample nucleic acid, which originates from an environmental sample, such as water, soil cyanobacterial bloom, cyanobacterial culture, mixed population of cyanobacteria and other microbes etc. Nucleic acid is usually DNA, but in can be also RNA. The nucleic acid is usually extracted from the sample by conventional means known for the skilled artisan, but may also be liberated by repeated freeze-thawing to disrupt cellular integrity, or cells are used directly from the sample.

These techniques may also comprise the step of amplifying the nucleic acid before analysis. Amplification techniques are known to those of skill in the art and include, but are not limited to cloning, polymerase chain reaction (PCR), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P. M. et al., 1988, Bio/Technology 6:1197).

The oligonucleotides of this invention are brought into contact with the target or sample nucleic acid under suitable conditions, which depend on the chosen molecular biology method, such as hybridization, PCR, LDR etc.

By “an oligonucleotide designed to be specific for the mcyE gene” it is meant that by using nucleic acid sequence data from several cyanobacterial genera and from several species of the genera, an oligonucleotide is designed to be specific for the mcyE gene of the microcystin synthetase operon. The length of an oligonucleotide may be 10 to 150 nucleotides depending on the detection method used. An oligonucleotide for hybridization is at least 20 bp, for PCR at least 10 bp and for LDR at least 15 bp.

Any probe or primer can be prepared according to methods well known in the art and described, e.g., in Sambrook, J. Fritsch, E. F., and Maniatis, T. (1989 (Molecular Cloning: A Laboratory Manual, Cold Spring Harbor Laboratory Press, Cold Spring Harbor, N.Y. For example, discrete fragments of the DNA can be prepared and cloned using restriction enzymes. Alternatively, probes and primers can be prepared using the Polymerase Chain Reaction CPCR) using primers having an appropriate sequence.

Primers and probes (RNA, DNA) described herein may be labeled with any detectable reporter or signal moiety including, but not limited to radioisotopes, enzymes, antigens, antibodies, spectrophotometric reagents, chemiluminescent reagents, fluorescent and any other light producing chemicals. Additionally, these probes may be modified without changing the substance of their purpose by terminal addition of nucleotides designed to incorporate restriction sites or other useful sequences.

These probes may also be modified by the addition of a capture moiety (including, but not limited to para-magnetic particles, biotin, fluorescein, dioxigenin, antigens, antibodies) or attached to the walls of microtiter trays to assist in the solid phase capture and purification of these probes and any DNA or RNA hybridized to these probes. Fluorescein may be used as a signal moiety as well as a capture moiety, the latter by interacting with an anti-fluorescein antibody.

By “a fragment of the mcyE gene” is meant principally any fragment of the mcyE gene which makes it possible to prepare oligonucleotides capable of identifying the mcyE gene from all of the microcystin producing genera and on the other hand is capable of discriminating different cyanobacterial genera from each other. The fragment is preferably related to the region of mcyE gene responsible for adding Adda and D-glutamate to the immature synthesis product. In particular, the fragment is related to the region catalyzing a peptide synthesis between Adda-D-glutamate and dehydroalanine and to the adenylating region. More specifically, the fragment is related to the region encoding the end part of the adenylation domain, the phospho-pantetheine binding site and the beginning of the domain which catalyses a peptide bond between D-glutamate and dehydroalanine. The length of the fragment may be between about 500 to 1000 nucleotides, which makes the alignment of nucleic acid sequence data from several cyanobacterial genera and species moderate to handle.

Examples of suitable fragments are the sequences of SEQ ID NO. 1 to SEQ ID NO: 34 as shown in FIG. 19 A to H or the consensus sequences SEQ ID NO: 35 to SEQ ID NO: 39 as shown in FIG. 15 A to C.

By “a fragment of the mcyD gene” is meant principally any fragment of the mcyD gene which makes it possible to prepare oligonucleotides capable of identifying the mcyD gene from all of the microcystin producing genera and on the other hand is capable of discriminating different cyanobacterial genera from each other.

Examples of suitable mcyD fragments are the sequences of SEQ ID NO. 131 to SEQ ID NO: 149 as shown in FIG. 38 A to F.

By “a suitable molecular biology method” is meant the chosen molecular biology method suitable for the purposes of detecting toxic cyanobacteria. The method may be selected from the group comprising hybridization, PCR, QRT-PCR, LCR, LDR and minisequencing.

PCR refers to the method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications. With PCR, it is possible to amplify a single copy of a specific target sequence in genomic DNA to a level detectable by the device and systems of the present invention.

PCR oligonucleotide primers or probes may be derived from either strand of the duplex DNA. The primers or probes may consist of the bases A, G, C, or T or analogs and they may be degenerated at one or more chosen nucleotide position(s). The primers or probes may be of any suitable length and may be selected anywhere within the DNA sequences from selected sequences which are suitable. In order to produce primers to a mcyE PCR, the mcyE gene(s) is typically examined using a computer algorithm, which starts at the 5′ or at the 3′ end of the nucleotide sequence. Typical algorithms will then identify oligomers in pairs of defined length that are unique to the gene, have a GC content within a range suitable for hybridization, and lack predicted secondary structure that may interfere with hybridization. The number of oligonucleotide pairs may range from two to one million.

Minisequencing reaction refers to a type of single base extension sequencing reaction using sequence terminators. In certain embodiments, minisequencing reactions are performed in the substantial absence of free single nucleotides, to minimize or prevent polymerization of nucleic acid beyond the single nucleotide sequenced by the sequence terminator. In certain embodiments, sequence terminators are labeled with fluorescent dyes, so that each nucleotide (A, G, T, or C) is identifiable by the color of the fluorescent label.

QRT-PCR or quantitative real-time PCR method involve measuring the amount of amplification product formed during an amplification process. Fluorogenic nuclease assays are one specific example of a real time quantitation method that can be used to detect and quantitate transcripts of present invention. In general such assays continuously measure PCR product accumulation using a dual-labeled fluorogenic oligonucleotide probe, an approach frequently referred to in the literature simply as the “TaqMan” method. The probe used in such assays is typically a short (ca. 20-25 bases) polynucleotide that is labeled with two different fluorescent dyes. The 5″ terminus of the probe is typically attached to a reporter dye and the 3″ terminus is attached to a quenching dye, although the dyes can be attached at other locations on the probe as well. For measuring a mcyE transcript, the probe is designed to have at least substantial sequence complementarity with a probe binding site on a mcyE transcript. Upstream and downstream PCR primers that bind to regions that flank mcyE are also added to the reaction mixture for use in amplifying the mcyE polynucleotide. When the probe is intact, energy transfer between the two fluorophors occurs and the quencher quenches emission from the reporter. During the extension phase of PCR, the probe is cleaved by the 5″ nuclease activity of a nucleic acid polymerase such as Taq polymerase, thereby releasing the reporter dye from the polynucleotide-quencher complex and resulting in an increase of reporter emission intensity that can be measured by an appropriate detection system.

Hybridization is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. For example stringent hybridization conditions are defined in Sambrook et al. 1989.

Ligation Detection Reaction LDR is based on the discriminative properties of the DNA ligation reaction. It requires the design of two probes specific for each target sequence, as described by Barany and co-workers (1999). One oligonucleotide brings a fluorescent label or other detection label and the other a unique sequence named complementary Zip Code (cZip Code). Ligated fragments, obtained in the presence of a proper template by the action of a DNA ligase, are addressed to the location on the microarray where the Zip Code sequence has been spotted. Such an array is therefore “Universal” being unrelated to a specific molecular analysis.

When two complementary pairs of probe elements are utilized, the process is referred to as the ligase chain reaction which achieves exponential amplification of target sequences (F. Barany, “The Ligase Chain Reaction (LCR) in a PCR World,” PCR Methods and Applications, 1:5-16 (1991)).

As used herein “Arrays” or “Microarrays” refers to an array of distinct polynucleotides or oligonucleotides synthesized on a substrate, such as paper, nylon or other type of membrane, filter, chip, glass slide, or any other suitable solid support. The microarray can be prepared and used according to the methods described, for example in Lockhart, D. J. et al. (1996; Nat. Biotech. 14: 1675-1680) and Schena, M. et al. (1996; Proc. Natl. Acad. Sci. 93: 10614-10619).

The microarray or detection kit is preferably composed of a large number of unique, single-stranded nucleic acid sequences, usually either synthetic antisense oligonucleotides or fragments of cDNAs, fixed to a solid support. The oligonucleotides are preferably about 6-60 nucleotides in length, more preferably 15-30 nucleotides in length, and most preferably about 20-25 nucleotides in length. For a certain type of microarray or detection kit, it may be preferable to use oligonucleotides that are only 7-20 nucleotides in length The microarray or detection kit may contain oligonucleotides that cover the known 5′, or 3′, sequence, sequential oligonucleotides which cover the full length sequence; or unique oligonucleotides selected from particular areas along the length of the sequence. Polynucleotides used in the microarray or detection kit may be oligonucleotides that are specific to a moyE gene or genes of interest.

The nucleotide sequence data can be aligned and clustered according to their phylogenetic lineages so that “group-specific” consensus sequences are yielded: Anabaena, Microcystis, Nodularia, Nostoc, Oscillatoria/Planktothrix. Then, “group-specific” probes can be designed using a suitable database, such as ARB database named “robe design”. Among the set of probes, discriminating probes with 3′ position unique to each group in order to obtain ligase discrimination can be selected. After hybridization of a discriminating probe and a common probe to the target sequence, ligation occurs only if there is perfect complementarity at the junction between the two oligos. Common probes are designed immediately 3′ to the discriminating oligo from the group-specific consensus and the detection is made by microarray method.

Zip code sequences can be selected randomly from those described by Chen and co-workers, 2000. Each Zip code is randomly assigned to a single cyanobacterial group. Each common probe is synthesized to have the complementary Zip code (cZip code) affixed to its 3′ end.

Examples of discriminating probes are SEQ ID NO: 40 to SEQ ID NO: 45 and of common probes SEQ ID NO: 46 to SEQ ID NO: 51 designed to be specific for mcyE gene.

Examples of LDR zip codes are zip codes SEQ ID NO: 52 to SEQ ID NO:57.

Furthermore, examples of discriminating probes are SEQ ID NO: 71 to SEQ ID NO: 90 and of common probes SEQ ID NO: 91 to SEQ ID NO: 110 designed to be specific for 16S rRNA gene.

The method of the present invention can be used to detect toxic cyanobacteria at least from the genera Anabaena, Microcystis, Planktothrix, Nostoc and Nodularia.

The method can be combined if desired with a detection method using oligonucleotides designed specific for any other mcy genes or for 16S rRNA gene. A method based on 16S rRNA gene detection is in particular useful, if non-toxic cyanobacteria should be identified in addition to toxic cyanobacteria, when for example the condition of environment is monitored.

The method of this invention can be combined also with methods determining microcystin concentration, cell density, cell number, biomass, biovolume, chlorophyll-a, total RNA/DNA concentrations etc.

A kit for the detection of toxic cyanobacteria by microarray method preferably comprises

discriminating and common probes designed to be specific for the mcyE gene;

DNA or RNA zip and complementary zip codes assigned to be specific for certain cyanobacteria genera.

A kit for the detection of toxic cyanobacteria by hybridization preferably comprises

primers designed to be specific for the mcyE gene;

probes designed to be specific for certain cyanobacteria genera.

In the kit can be in addition to primers or probes designed to be specific for the mcyE and/or mcyD gene also primers or probes designed to be specific for 16 S rDNA.

In this invention we have identified and characterized the genes for the biosynthesis of hepatotoxins, microcystins from the filamentous, nitrogen fixing cyanobacterium Anabaena strain 90. Microcystin synthetase genes are now known from three different cyanobacterial genera, Anabaena, Microcystis and Planktothrix, which are the main producers of the microcystins. The arrangement of the genes is different between these species. The order of the domains, which are coded by two sets of the genes, is co-linear with the hypothetical sequence of the enzymatic reactions for microcystin biosynthesis only in Anabaena 90.

These genes provide extensive sequence information for the design of primers to be used in PCR-based methods for the sensitive detection, identification and quantification of producers of hepatotoxic microcystins and nodularins.

Identifying the most potent microcystin producer in a lake could be valuable knowledge e.g. in designing lake restoration strategies. In connection of this invention we identified the microcystin producing genera and quantified the microcystin synthetase gene E (mcyE) copy numbers in two lakes (Lake Tuusulanjärvi and Lake Hiidenvesi) by quantitative real-time PCR. Microcystin concentrations and cyanobacterial cell densities of these lakes were also determined. The main microcystin producer in Lake Tuusulanjärvi was Microcystis sp., since average Microcystis mcyE copy numbers were over 30 times more abundant than those of Anabaena. Lake Hiidenvesi seemed to contain both nontoxic and toxic Anabaena as well as toxic Microcystis strains. Microcystin concentrations of Lake Tuusulanjärvi and Lake Hiidenvesi correlated positively with Microcystis mcyE copy numbers.

mcyE sequences from Anabaena, Microcystis, Nodularia, Nostoc and Oscillatoria/Planktothrix were used for detecting polymorphic positions useful for detecting cyanobacterial strains using several different biomolecular techniques. These unique features were used for designing probes for cyanobacterial detection and identification by LDR in combination with a microarray.

The molecular classification of cyanobacteria is based on 16S rRNA gene sequences obtained from pure cultures (Wilmotte & Herdmann, 2001). Using this molecular information, several techniques can be used to determine the cyanobacterial composition of an environmental sample. The most widely used method is the 16S rRNA gene amplification with cyanobacterial specific PCR primers, cloning, sequencing and phylogenetic reconstruction (Giovannoni et al., 1988). This strategy is very time consuming and therefore is not suited to large scale screenings. Recently, DGGE and TGGE have been widely applied to molecular ecological research (Muyzer, 1999). However, the excision of bands, reamplification and sequencing are necessary to obtain a precise diversity analysis.

Oligonucleotide microarrays (microchips) have a major role in genomics and have gained wide attention in molecular diagnostics. Microarray technology has a great potential in environmental diagnostics. In fact, the DNA microarray technology has already been applied for microbial diversity detection. Microarrays have been used for quantitation of target microbial populations for environmental analysis (Guschin et al., 1997).

Rudi and coworkers (2000) designed a small cyanobacterial specific microarray for Microcystis, Planktothrix, Anabaena, Aphanizomenon, Nostoc and Phormidiun.

DNA microarray and the magnetic-capture hybridization technique have been combined to form a new technology named MAG-microarray. Bacterial magnetic particles (B3 MPs) on a MAG-microarray have been used for the identification of cyanobacterial DNA (Matsunaga et al., 2001). Genus-specific oligonucleotides probes for the detection of Anabaena spp., Microcystis spp., Nostoc spp., Oscillatoria spp. and Synechococcus spp. have been designed from the variable region of the cyanobacterial 16S rRNA gene of 148 strains. These probes have been immobilized on BMPs via streptavidin-biotin conjugation and employed for magnetic-capture hybridization against digoxigenin-labeled cyanobacterial 16SrRNA gene. Bacterial magnetic particles have been magnetically concentrated, spotted in a microwell on MAG-microarray and detected. The entire process of hybridization and detection has been automatically performed and all the five cyanobacterial genera have been successfully discriminated.

Recently, we have presented a Universal DNA Array approach to discriminate some groups of bacteria (Busti et al., 2002). This procedure, based on the discriminative properties of the DNA ligation reaction, requires the design of two probes specific for each target sequence, as described by Gerry and co-workers (1999). One oligonucleotide brings a fluorescent label and the other a unique sequence named complementary Zip Code (cZip Code). Ligated fragments, obtained in presence of a proper template by the action of a DNA ligase, are addressed to the location on the microarray where the Zip Code sequence has been spotted. Such an array is therefore “Universal” being unrelated to a specific molecular analysis.

Here we present the Universal DNA Array approach applied to the detection of cyanobacterial diversity. We designed probes specific for 19 different cyanobacterial groups (phylogenetic lineages including Anabaena/Aphanizomenon, Calothrix, Cylindrospermopsis, Cylindrospermum, Gloeothece, Halotolerants, Leptolyngbya, Lyngbya, Microcystis, Nodularia, Nostoc, Oscillatoria/Planktothrix, Phormidium, Prochlorococcus, Spirulina, Synechococcus, Synechocystis, Trichodesmium, Woronichinia) identified from the phylogenetic tree obtained from the ARB database constructed in this study.

13 axenic strains from culture collection, 38 isolated culture strains and 44 clonal fragments recovered from environmental samples were used for validation purposes with excellent results demonstrating a high discriminative power. The proposed approach is extremely sensitive (down to 1 fmol of PCR amplified 16S gene region are detectable) allowing for the analysis of unbalanced environmental samples. LDR coupled to Universal Microarray performed on PCR samples containing 100:1 ratios of different amplicons yielded the correct identification of the starting strains. This approach is therefore amenable to the analysis of complex environmental samples.

The Universal array was used for the detection of toxic and non-toxic cyanobacteria by using probes designed to detect both the 16 rRNA and mcyE genes. In the presence of the proper DNA template of both 16S rRNA and mcyE genes, the Universal Array functioned very well: only group specific spots, universal spots and the spots corresponding to the hybridization control showed positive.

Genes Coding for the Synthesis of Microcystins in Anabaena

The Order of the Genes in the Microcystin Synthetase Gene Cluster is Different in the Cyanobacterial Species

The arrangement of the genes is different in the gene clusters of microcystin biosynthesis from the strains of three species. In Anabaena strain 90, Microcystis aeruginosa (Tillett et al., is 2000; Nishizawa et al., 2000) and in Planktothrix agardhii CYA126 (Christiansen et al., 2003) the NRPS genes, mcyA, mcyB and mcyC have the same order, but the organization of the other genes is different. In Anabaena strain 90 and in M. aeruginosa the mcy-genes are in two clusters, which are transcribed in opposite directions, whereas in P. agardhii they are in one cluster transcribed in the same direction (except mcyT, which was not found in Anabaena and Microcystis). The arrangement of the genes from mcyD to mcyH in Microcystis is almost identical in Planktothrix (mcyF is missing in Planktothrix), but it differs from the order in Anabaena. In Planktothrix, compared to Microcystis, the part containing mcyD, mcyE, mcyF, mcyG, mcyH, mcyI and mcyJ is reversed. In this rearrangement, mcyF and mcyI were lost from the cluster and mcyJ was relocated after mcyG

The Biosynthesis of Microcystins

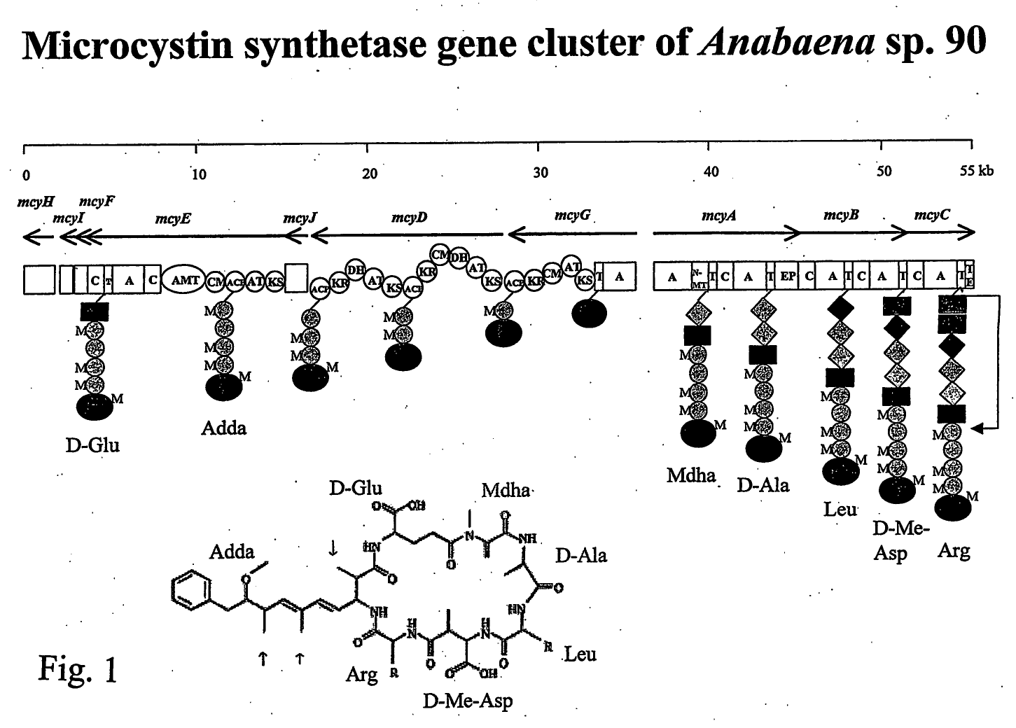

In Anabaena, the order of the domains coded by the genes in the two sets is co-linear with the hypothetical sequence of the enzymatic reactions for microcystin biosynthesis (FIG. 1). The progression of the biosynthetic reactions follows the order of the functions coded first by mcyG and continuing with the activities coded by mcyD, mcyJ, mcyE, mcyF, mcyI, mcyA, mcyB and mcyC.

Phenyl acetate is the assumed starting unit in the biosynthesis of Adda (Moore et al., 1991). It is activated by the adenylating domain identified in the N-terminus of McyG, and transferred onto the subsequent thiolation (phosphopantetheine binding) site. Polyketide synthesis reactions are followed (FIG. 1). All four extension units are malonyl-CoA molecules according to the substrate specificity of the AT domains (FIG. 4). In McyG there is a KS domain to catalyse the first condensation reaction between phenylacetate and malonyl-CoA.

The reductive reactions needed to fashion the polyketide chain are putatively catalysed by KR and DH domains of McyD and McyE. The KR domain of McyG is in the right position to reduce the carbonyl group of the putative starter molecule. The methyltransferase domains of McyG, McyD and mcyE are the obvious candidates to introduce three methyl groups into the carbon frame of Adda. It was recently verified with a knockout mutant (Christiansen et al., 2003) that the incorporation of the fourth methyl, which is seen in the methoxy group of Adda, is catalysed by McyJ. The amino transferase domain of mcyE most likely adds the amino group, which participates in the peptide bond with the glutamate residue.

There are two condensation domains of peptide synthetases in McyE. The first one logically catalyses the peptide bond between Adda and glutamate, which is activated by the adenylation domain of McyE. The signature sequence, which was also determined as DPRHSGVVG for mcyE of both M. aeruginosa and P. agardhii, has no precedents in the databases (Table 2). The synthetases of other peptides, which contain glutamyl residues are known for bacitracin, fengycin and surfactin (accession numbers: AF007865, AF023464, AF087452 and D13262). In these compounds the standard α-carboxyl of glutamate is part of the peptide bond, while in microcystins it is the γ-carboxyl. This is analogous to the activation of aspartate/methylaspartate by the second adenylation domain of McyB. The β-carboxyl of aspartate/methylaspartate instead of the α-carboxyl is engaged in the peptide bond formation. This must have impact on the compositions of the glutamate and aspartate/methylaspartate binding pockets in the adenylation domains.

McyA has two adenylation domains for the activation of serine and alanine, respectively. The signature sequences of these domains have models and are almost identical in Anabaena 90, M. aeruginosa and P. agardhii (Table 2). The dehydration of serine supposedly takes place after the activation by adenylation and is catalysed by McyI, which is similar to phosphoglycerate dehydrogenases.

There is only one, internal, condensation domain in McyA, which most likely links dehydroserine and D-alanine. The bond between glutamate and dehydroserine is putatively catalysed by the C-terminal condensation domain of McyE. There is a methyltransferase domain in the first module of McyA for N-methylation of dehydroserine. The epimerase domain at the C-terminus of McyA converts L-alanine to the D-form.

Two modules of McyB and one module of McyC logically activate, and add three residues to the nascent peptide chain: L-leucine or L-arginine, methylaspartate or aspartate and L-arginine, respectively (FIG. 1). The amino acids activated by the adenylation domains of McyC and by the first module of McyB (McyB-1) vary most frequently in microcystins. M. aeruginosa PCC7806 and M. aeruginosa K-139 produce mainly Mcyst-LR, and the substrate specificity conferring sequences in McyB-1 of these strains are identical with the signature sequence for leucine (Table 2). M. aeruginosa UV027 and P. agardhii CYA126 produce mostly Mcyst-RR, which is also produced by Anabaena 90 together with Mcyst-LR. Their signature sequences in McyB-1 are different and have no precedents in the databases (Table 2). In M. aeruginosa UV027 the specificity codes of McyB-1 and McyC are almost identical (DVWTIGAVE/DWTIGAVD) and match with the codes of McyC from M. aeruginosa K-139 and M. aeruginosa PCC7806, respectively (Table 2). Accordingly McyB-1 of M. aeruginosa UV027 and McyC activate arginine.

There is no epimerase domain in McyB of Anabaena 90 or in the other sequenced versions of McyB, though in microcystins, the aspartyl or methylaspartyl moiety is in the D-form. The epimerization in this position and in the glutamyl residue is putatively catalysed by McyF, which in a BLAST search was similar to aspartate racemases, and was shown by Nishizawa et al., (2001) to complement a D-glutamate deficient mutant of Escherichia coli. The C-terminal thiosterase domain of McyC, as generally in nonribosomal peptide synthesis, (Kohli et al., 2001) catalyzes the final step in microcystin biosynthesis, the cyclization of the linear peptide (FIG. 1).

McyH is probably not needed for the synthesis of microcystins but it may participate in the transport of microcystins.

In connection of this invention we obtained DNA sequences of three microcystin synthetase genes: mcyA, mcyE and mcyD. The mcyA gene fragment encodes part of the condensation domain, which catalyses a condensation reaction to form a peptide bond between the growing peptide and D-alanine. The fragment of the mcyE gene codes for a partial adenylation domain and a phospho-pantetheine-binding site, the region, which activates glutamic acid. The region of the mcyD gene encodes parts of both the β-ketoacyl synthase and the acyltransferase domains. We sampled representative producers of microcystins and nodularins (Table 1) in the genera Anabaena, Microcystis, Planktothrix, Nostoc, and Nodularia. Individual topologies generated from mcyA, mcyE and mcyD were rooted with homologues identified in BLAST searches. These topologies were congruent with one another (data not shown) and thus the data from all three genes were concatenated in order to increase the amount of information available in phylogenetic analyses.

Phylogenetic Evidence for the Early Evolution of Microcystin Synthesis

In order to investigate the role of horizontal gene transfer in the distribution of microcystin synthetase genes amongst cyanobacteria we assembled a data set comprised of 16S rRNA and rpoC1 sequences from the same set of taxa. These genes are conserved and widely used as tools for phylogenetic classification. No incongruence between the 16S rRNA and rpoC1 topologies could be found and the sequence data of these two genes was concatenated. We analysed these two data sets separately with maximum parsimony and maximum likelihood optimisation criteria. Bootstrap analyses were conducted to measure the stability of the observed phylogenetic patterns and revealed two well-supported topologies (FIG. 7). The two maximum-likelihood topologies were perfectly congruent (FIG. 7). The bootstrap support for the monophyly of the genera Anabaena, Nodularia and Nostoc was lower in the microcystin synthetase gene data set than in the 16S rRNA and rpoC1 data set (FIG. 7). Likewise the bootstrap support for the monophyly of the genera Planktothrix and Microcystis was lower in the 16S rRNA and rpoC1 data set than in the microcystin gene data set (FIG. 7). However, no conflicting nodes received bootstrap support above 45% in any analysis. Individual trees generated from mcyA (26 taxa), mcyE (30 taxa) and mcyD (19 taxa) all consistently supported the reciprocal monophyly of each genus (data not shown). In no instance was support for a lateral transfer recovered. The high degree of congruence between the microcystin synthetase gene data set and 16S rRNA and rpoC1 data set is consistent with an ancient origin of microcystins (FIG. 7). This indicates that the phylogenetic marker genes and the microcystin synthetase genes have co-evolved for the entire length of the evolutionary history of this toxin. The sporadic distribution of microcystin synthetase genes in modern cyanobacteria suggests that the ability to produce the toxin has been lost repeatedly in the more derived lineages of cyanobacteria. Microcystins are one of the few known natural examples of combined polyketide synthase and peptide synthetase systems. Little is known about the evolution of these mixed polyketide and peptide synthetases and it is unclear whether the combination of these two systems is of recent origin. Congruence between the polyketide and peptide portions of the gene cluster as well as the 16S rRNA and rpoC1 data set demonstrates that the combination of these two systems is an ancient collaboration in the production of this toxin. Our results do not rule out the possibility that parts of the sequences of the microcystin synthetase gene cluster are of more recent origin. Indeed, the existence of many microcystin variants implies a fast evolution of certain gene domains.

Similarities in the chemical structures and biological action of microcystins and nodularins indicate that these compounds are closely related (Sivonen and Jones, 1999). However, the exact relationship between nodularins and microcystins remains ambiguous. Recent studies have suggested that the genes encoding microcystin synthetase have evolved from the genes encoding nodularin synthetase (Christiansen, 2003). Our data rejects the idea that nodularin synthesis predates microcystin synthesis (Christiansen, 2003 or that nodularin synthetase genes are a sister group to microcystin synthetases genes (Moffitt et al. 2001). Instead, our results suggest that nodularin synthetase genes are derived from microcystin synthetase genes and that nodularins should now be regarded as structural variants of microcystins. It is anticipated here that nodularin synthetase genes were formed from the ancestral microcystin synthetase gene set through a relatively recent deletion of the last mcyA module and the first mcyB module and by mutation changing the substrate specificity coded by the first module of mcyA. This finding is consistent with the production of nodularins by a single cyanobacterial genus and the limited structural variation of nodularins in comparison to microcystins Sivonen and Jones, 1999). Microcystins are commonly believed to have evolved in response to grazing pressure by zooplankton (DeMott et. al. 1991). Fossils of filamentous akinete-forming cyanobacteria are dated to 2000 million years ago (Amard et al., 1997).

This means that the Anabaena, Nostoc, and Nodularia genera and thus, the common ancestor of microcystin producing cyanobacteria are at least this old. Molecular clocks set a divergence time of 1576 million years ago for the crown eukaryotic lineages (Heckman, D. S. et al., 2001). Metazoans such as copepods and cladocerans are often envisaged as target organisms of microcystins (DeMott and Moxter, 1991). However, microcystin production predates all metazoans. If microcystins evolved as a chemical defense against zooplankton then the targets of the toxin must have been the early branching eukaryotes (Moon-van der Staay, S-Y. et al., 2001 and Brocks et al., 1999).

Protozoans are an underappreciated component of the zooplankton and may have been overlooked as the likely targets for the evolution of chemical defense in this case. It is not clear that microcystins evolved as a chemical defense and other proposed functions for microcystins include siderophobic scavenging of trace metals such as iron (Utkilen and Gjolme, 1995) and a role in signalling and gene regulation (Dittmann et al, 2001).

Microcystins and nodularins are highly toxic to eukaryotic cells and pose a serious health risk to water users. Also the genera Arthrospira and Aphanizomenon are commonly used in health food supplements (Gilroy et al., 2000). Our study demonstrates that the ability to make microcystins has been lost repeatedly throughout the diversification of cyanobacteria. This means that toxin-producing strains may be found unexpectedly.

Quantification of Microcystin Synthetase E Copy Numbers of Microcystis and an Anabaena in Lakes by Quantitative Real-Time-PCR

In this invention a novel method to indicate the main putative microcystin producer of a lake is provided. The dominant putative microcystin producer was Microcystis in Lake Tuusulanjärvi and in the Basin of Kiihkelyksenselkä of Lake Hiidenvesi based on mcyE copy number quantification. This method enables to study in situ the responses of environmental factors on the growth of microcystin producing genera and could be used to observe the possible changes in cyanobacterial assemblages prior, during, and after lake restoration in order to find out, if the genus targeted lake restoration succeeded.

The Main Microcystin Producers

In Lake Tuusulanjärvi Microcystis spp. was the main putative microcystin producer, since average Microcystis mcyE copy numbers were clearly higher than those of Anabaena and thus, this result was in agreement with the higher cell numbers of Microcystis observed compared to those of Anabaena. Microcystin concentrations or hepatotoxicities have also presiously correlated positively with Microcystis spp. biomass in Lake Tuusulanjärvi (Ekman-Ekebom et al. 1992, Lahti et al. 1997). Microcystis spp. were also the main putative microcystin producers in the Basin of Kiihkelyksenselkä of Lake Hiidenvesi, although Anabaena cell numbers were higher than those of Microcystis. This indicates that majority of the Anabaena cells were nontoxic and Microcystis cells toxic in this basin. In the Basins of Mustionselkä, Nummelanselkä and Kirkkojärvi of Lake Hiidenvesi the main microcystin producer could not be assessed, since in the Basins of Mustionselkä and Nummelanselkä, the Anabaena and Microcystis mcyE copy numbers were quite similar and in the Basin of Kirkkojärvi the Anabaena and Microcystis mcyE copy numbers were below the detection limit. The low mcyE copy numbers detected in Kirkkojävi were in agreement with the low microcystin concentrations measured from this basin. Microcystin concentration correlated positively with Microcystis mcyE copy numbers with all studied samples whereas no significant correlation was found between microcystin concentrations and Microcystis and Anabaena cell numbers with all studied samples. Therefore, with microscope analysis it is not possible to determine reliably the most potent microcystin producer of a lake. Gene mcyE copy numbers, microcystin concentrations, and cyanobacterial cell densities were lower in Lake Hiidenvesi than in Lake Tuusulanjärvi. In Lake Tuusulanjärvi and in surface water of the Basins Nummelanselkä and Kiihkelyksenselkä of Lake Hiidenvesi WHO microcystin concentration guideline value for drinking water quality, 1 μg 1⁻¹, (Falconer et al., 1999.) was exceeded.

Microcystis and Anabaena mcyE copy numbers were one to over 200 times higher than the cell numbers observed with microscopy in Lake Tuusulanjärvi and Lake Hiidenvesi. In Lake Tuusulanjärvi Microcystis mcyE copy numbers increased after August in contrast to the cell density, which decreased. The explanation could be that after August cells had more genome copies or that the DNA of the lysed cells was present in the lake water and followed through the cell concentration and DNA extraction processes to the final DNA sample. Additional explanations for the high mcyE copy number and cell density ratio might be that the cell numbers detected with microscope were too low or the genome sizes of the external standard strains were underestimated. Even with the knowledge that cyanobacteria may have several genome copies in a cell (Becker, et al. 2002, Herdman et al., 1979, Labarre et al., 1989), it seems that the obtained mcyE copy numbers were too high. The genome sizes estimated for the Anabaena standard strains were 5.15 Mb according to the published data of Anabaena PCC 6309 and PCC 7122 (Castenholz, 2001). These Anabaena strains are nontoxic (Lyra, et al. 2001) and lack the microcystin synthetase genes, the sizes of which are not more than 53 or 55 kb (Christiansen et al., 2003, Nishizawa et al., 2000 and Nishizawa et al. 1999 and Trlnett et al. 2000 and Example 1). For Microcystis standard strains the genome size of 4.70 Mb was used according to the genome size of one of the external standard strains, Microcystis PCC 7941 (Castenholz, 2001).

In general, nontoxic strains do not contain mcy genes (Neilan et al., 1999 and Tillett et al. 2001). However, some strains may have fragments of microcystin synthetase genes or mutations within these genes (Kaebernick et al. 2001, Neilan et al. 1999 and Tillett et al. 2001). These strains can be amplified with may primers, although they are not able to produce toxins. However, the significant positive correlation between Microcystis mcyE copy numbers and microcystin concentration indicated that such nontoxic strains were probably not present in Lake Tuusulanjärvi and in Lake Hiidenvesi.

Amplification efficiency. Microcystis mcyE QRT-PCR amplification efficiencies with Lake Tuusulanjärvi water samples (0.78-0.99) were similar to those of Microcystis standards (0.86-0.94) and those of Anabaena standards (0.96-0.99), which is a prerequisite for correct mcyE copy number quantification of the lake water samples. These similar QRT-PCR amplification efficiencies also ensured that no PCR-inhibiting contaminants were present in the Lake Tuusulanjärvi DNA samples. However, Anabaena mcyE QRT-PCR amplification efficiencies with Lake Tuusulanjärvi water samples were higher than one. This result can be explained by competition for primer annealing sites between primers and homologous sequences (Becker et al. 2000, Suzuki et al. 1996, Wawrik et al. 2002) and this competition may lead to suppression of the target DNA (Suzuki et al. 1996). This phenomenon has been shown to occur not only in conventional PCR (Suzuki et al. 1996) but also in QRT-PCR (Becker et al. 2000, Wawrik et al. 2002), although quantification is achieved during the early logarithmic phase of the amplification (Heid et al., 1996). Anabaena and Microcystis mcyE sequences are homologous (Example 2). Since in Lake Tuusulanjärvi the concentration of competing Microcystis mcyE genes was higher than that of Anabaena mcyE genes, it is possible that the Anabaena mcyE copy numbers were underestimated. In addition, the mcyE-F2 forward primer amplified Anabaena as well as Microcystis sequences and increased the amount of competing homologous sequences.

Detection range of mcyE copy number quantification. The mcyE QRT-PCR amplification was log-linear in a range of three to four orders of magnitude. With high DNA template concentration, 6.6×10⁶ mcyE copies in a reaction, amplification was inhibited with the DNAs of Anabaena 90, Anabaena 202A1, Microcystis GL 260735, and Microcystis PCC 7941 strains, since obtained Ct values were lower than they should have been according to the regression equation or Ct values could not be detected at all. The inlubition was probably caused by contaminants that co-extracted with DNA during the DNA extraction and purification as shown previously (Wintzingerode et al. 1997). The lowest detection limit of Anabaena and Microcystis mcyE QRT-PCR amplification was 660 mcyE copies in a reaction. The error of the Ct values in QRT-PCR has been shown to be higher with low DNA template concentrations than with high template concentrations (Grüntzig et al. 2001). However, in this study the lowest mcyE copy number concentrations of the external standards had the same CV % as the other concentrations, 0.1-3.6%.

The utilization of the mcyE copy number results. In this study, putative microcystin producing Anabaena and Microcystis were detected in both studied lakes. In Lake Tuusulanjärvi and in the Basin of Kiihkelyksenselka of Lake Hildenvesi the dominant putative microcystin producer was Microcystis based on mcyE quantification. Reduction of nutrient loading and resuspension (Boers et al. 1991, Chorus and 1999, Reynolds, 1997) could be successfiul strategies to decrease the density of Microcystis, since these may decrease nitrogen as well as phosphorus concentrations of the water. In addition, lower nutrient concentrations could favor the growth of nontoxic Microcystis strains instead of toxic, since the biomass of nontoxic Microcystis strains has been demonstrated to be higher than that of toxic strains with low nutrient concentrations at the end of a laboratory experiment (Vezie et al. 2002). Lake Hiidenvesi seemed to have nontoxic and toxic Anabaena strains as well as toxic Microcystis strains. However, mcyE copy numbers should be monitored during the whole growth period in order to have a better understanding of the population dynamics of this lake. A reduction of the external phosphorus loading could affect the mass occurrences of nitrogen-fixing cyanobacteria negatively. It is however not known how the reduction of nitrogen fixlng-cyanobacteria would affect the growth of toxic Microcystis strains. At least, the presence of toxic Microcystis strains should be taken into account in land use management of the catchment area of Lake Hiidenvesi.

Oligonucleotides for Detection and Identification of Toxic Cyanobacteria

In this invention was developed the identification on mcyE gene region of polymorphisms specific for different toxic cyanobacterial groups identified from the phylogenetic tree obtained from 34 toxic cyanobacterial sequences. The polymorphic positions were used for designing probes for PCR, hybridization, primer extension, ligation and LDR. Probes for ligation have been used in combination with randomly chosen tag sequences appended 5′ to the so called common primers in order to be used in the universal array approach. Validation against different samples demonstrate the robustness of the proposed polymorphisn and probes.

Molecular Analysis of Cyanobacterial Diversity by Microarrays on “PCR-Amplified” 16SrRNA gene

We aimed at designing and testing a microarray based system for cyanobacterial diversity identification. We selected a molecular strategy based on the amplification of the 16S rRNA gene region using cyanobacteria specific primers (Edwards et al. 1989, Lepre et al. 2000) followed by group discrimination based on a multiplexed ligation detection reaction performed employing proper probes. Ligated fragments characteristics of each group were demultiplexed on a Universal array. This approach, originally proposed by Gerry et al (1999) has found several application. We used the ARB database including 281 public sequences belonging to the 19 phylogenetic lineages we decided to target (Anabaena/Aphanizomenon, Calothrix, Cylindrospermopsis, Cylindrospermum, Gloeothece, Halotolerants, Leptolyngbya, Lyngbya, Microcystis, Nodularia, Nostoc, Oscillatoria/Planktothrix, Phormidium, Prochlorococcus, Spirulina, Synechococcus, Synechocystis, Tnichodesmium, Woronichinia). Not all of these groups are present in the environmental samples from the lakes involved in the MIDI-CHIP project but all them were included in order to allow for future research studies. Sequences were clustered as shown in FIG. 25. For each group we calculated a consensus sequence with a cutoff of 75%. The resulting consensuses were aligned and group specific probes were searched along the entire 16S rRNA gene region. Following the LDR approach (FIG. 26) we identified two unique probes for every group (a common probe and a discriminating probe). Selected probes were tested against the set of sequences of the corresponding group in order to verify the perfect match, in particular around the site of ligation. Then probe sequences were tested against the remaining cyanobacterial sequences in order to verify their selectivity. Selected probes are spread all over the entire 16S amplicon. Selected common probes were then randomly combined to a set of cZipCode sequences previously proposed for the Universal array approach (Gerry et al 1999, Chen et al 2000). Potential cross hybridization was checked by BLAST analysis of each common and discriminating probe against all others. Probes were then synthesized, HPLC purified and tested by mass spectrometry. This stringent quality assurance procedure is mandatory to achieve expected results in LDR. Ordinary PCR quality probes yielded poor performance due to low phosphorilation or Cy3 labeling and exceedingly high failure sequences. Similar quality controls were performed on the 5′ amino-modified ZipCode sequences spotted by contact printing on Codelink Slides. We generated 8 subarrays per slide (96 spots per subarray including zipcodes for a hybridization control (eight spots at corners), cyanobacterial universal probes (12 spots in the middle and at corners) and 19 lineage-specific ZipCodes spotted in quadruplicate. Slides were batch-tested by hybridization using a labeled polyT probe matching the polyA tail appended in 5′ to every ZipCode probe. In order to validate the designed probes we run a blank (no template) LDR. No signals were detected demonstrating that no false ligation occurred (this problem is often encountered when performing minisequencing (Lindroos, 2002). Then 51 strains of known 16S rRNAsequence belonging to 13 phylogenetic groups (FIG. 33) were used to test the proposed system. FIG. 28 clearly illustrates LDR specificity when using 100 fmol of each single template independently reacted against the complete set of probes. Six out of 19 groups were not included in the test panel due to their unavailability but their corresponding LDR probes were present in the LDR mix and did not generate any false positive result. It should be noted that, although not identical, the LDR/Universal array efficiency was very similar among all probes. Comparing the intensity between the cyanobacterial universal probe and each lineage specific probe, we found a ratio very close to 1 for most groups. (Here a graph showing this comparison could be more clear that the following-description). Probes for Lyngbya, Nodularia, Anabaena and Cyanotizece (FIG. 28 D, F, N, O respectively) consistently yielded higher efficiency. However the similarity of results using very different sequences having very close thermodynamic properties is a distinctive feature of this approach. Hybridization based arrays Loy, 2002; Rudy K. 2000) depend heavily on local sequence characteristics. When hybridization is performed in high salt buffers in a single stringency condition, large variability in signal intensity can be expected (Loy, 2002). On the contrary, using the exquisite sequence specificity of the ligation reaction (Gerry, 1999) and the very high annealing temperatures required during cycling, a very homogeneous behaviour is found. Very little influence of the sequence context has been demonstrated. Our results in a different sequence context, the highly polymorphic HLA region (Consolandi, 2003) further confirm these findings. Another distinctive feature of the LDR approach is related to the excellent sensitivity gained by means of a cycling procedure based on thermostable ligases. We were able to detect down to 1 fmol (around 2 ng) of PCR amplified material thanks to the linear amplification gained through LDR. FIG. 31 show the results we obtained using a serial dilution of Planktothrix 16S amplicon from 100 fmol to 1. A good linear relationship was found plotting the signal intensity against the concentration in a log scale.

The Universal array was used for the detection of toxic and non-toxic cyanobacteria designed to detect both the 16 rRNA and mcyE gene ligated probes. The ligation detection reaction was carried out under the same conditions by using an oligo mix containing both the probes for 16S rRNA gene and the probes for the mcyE gene. Finally the hybridization was carried on the same Universal Array where the 16S rRNA LDR product and, mcyE LDR product were detected.

### EXAMPLES

### Example 1

Genes Coding for the Synthesis of Hepatotoxic Heptapeptides (Microcystins) in the Cyanobacterium Anabaena Strain 90

Bacterial Strains and Culture Conditions

The cyanobacterial strain Anabaena 90 was isolated from Lake Vesijärvi, Finland and purified axenic (Sivonen et al., 1992; Rouhiainen et al., 1995). It was shown to produce three microcystins (MCYST-LR, MCYST-RR and D-Asp-MCYST-LR (Sivonen et al., 1992). Anabaena strain 90 was grown in Z8 medium (Kotai, 1972) without nitrate at ˜22° C. with continuous illumination of 20-25 μmol m⁻²s⁻¹. Escherichia coli strain DH5 α, which was used as a host for DNA cloning and sequencing, was cultured in Luria Broth at 37° C.

DNA Manipulations, Sequencing, Screening and Mapping of Cosmids

Extraction of cyanobacterial DNA and the preparation of genomic library has been described earlier (Rouhiainen et al. 2000). The genomic library was screened by colony hybrdization (Sambrook et al., 1989). The probe labelled with [³²P]dCTP was a 2.5 kb fragment from mcyA of Microcystis aeruginosa provided by Dr. Elke Dittmann (Humboldt University, Berlin). A total of about 6,000 colonies were tested. The insert DNA of 29 positive cosmid clones was mapped with HindIII, EcoRI and SpeI. The ends of 18 inserts were sequenced with SP6 and T7 primers, and the cosmid clones for sequencing the microcystin synthetase genes were selected. DNA of the cosmid clones was digested with restriction enzymes BstEII, HindIII, EcoRI, ScaI, SpeI or XbaI and ligated to pBluescript SK(+). Nested deletions and other DNA manipulations were performed according to Sambrook et al., (1989). Sequencing was carried out mainly by the University of Chicago Cancer Research Center DNA Sequencing Facility. Gaps were filled by amplifying chromosondal DNA in PCR with DyNAzyme™ EXT Polymerase (Finnzymes), the sequencing reactions were done with the BigDye Terminator Cycle Sequencing Kit (Applied Biosystems) and analyzed on the ABI 310 Genetic Analyzer. The standard T3 and T7 primers and oligonucleotides derived from already determined sequences were employed.

Sequence Analysis

Analysis and comparisons of sequences were performed with the Sequence analysis software package, version 8.0, University of Wisconsin Genetics Computer Group and with EMBOSS (European Molecular Biology Open Software Suite). CAP program (http://bioweb.pasteur.fr/seganal/interfaces/cap.html) was used for sequence assembly. Sequence similarity searches in databases were done with BLAST through the website of the National Center for Biotechnology Information http://www.ncbi.nlmnih.gov/BLASI). Searches for conserved domains and motifs were accomplished with the CD-Search program (http://www.ncbi.nlm.nih.gov/Structure/cdd/cdd.sht and with the Motif Scan program (http://hits.isb-sib.ch/cgi-bin/PFSCAN?). Clustal W was applied for multiple sequence alignments (http://npsa-pbil.ibcp.fr/cgi-bin/npsa_automat.pl?page=npsa_clustalw.html).

Organization of the Microcystin Synthetase Genes

Microcystin synthetase genes in Anabaena strain 90 (mcyA-J) are organized in three putative operons (FIG. 1) with a total size of 55.4 Kb. The first operon (mcyA-mcyB-mcyC) is transcribed in the opposite direction compared to the second (mcyG-mcyD-mcyJ-mcyE-mcyF-mcyI) and the third operon (mcyH). The ORFs mcyA and mcyG are separated by 1275 bp; mcyI and mcyH by 297 bp (FIG. 1). The putative promoter regions were identified in front of mcyA (the −10 sequence, TAAATT, 315 bp and the −35 sequence, TTGTAT, 339 bp upstream from the translation start codon, ATG, of mcyA) and in front of mcyG (the −10 sequence, TATAAG, 145 or 223 bp and the −35 sequence, TTGACA, 172 or 250 bp upstream from the potential translation starts of mcyG). The promoter region was also identified before mcyH (the −10 sequence, TATAAA, 57 or 216 bp and the −35 sequence, TTGATA, 79 or 238 bp from the suggested translation initiation codons). Transcriptional starts prior to mcyD (distance 93 bp from mcyG), mcyE (37 or 95 bp from mcyJ), mcyF (42 bp from mcyE) and before mcyI (51 bp from mcyF) cannot be ruled out, although no transcription stop loops were identified following the preceding genes, and no Pribnow box could be identified in front of mcyD.

Characterization of the Peptide Synthetase Genes

In the first operon there are three open reading frames (ORFs) named mcyA, mcyB, and mcy. We suggest that the translation of mcyA starts with the ATG codon preceded (3 bp) by a potential ribosome binding site (RBS) GGAGAAG. The next ORF, mcyB, begins with an ATG codon 18 bp downstream from the previous stop codon (TAA) and 12 bp from a potential RBS AGAGGA. mcyC is overlapped by mcyB with one base pair. A putative RBS (ACGACAAG) is found 5 bp before the start codon ATG of mcyC. The lengths of mcyA, mcyB, and mcyC are 8364,6399 and 3852 bp and they encode polypeptides with predicted masses of 315,663, 243,072, and 146,877 Da, respectively. The sequence analysis of mcA, mcyB, and mcyC revealed a typical modular structure for nonribosomal peptide synthetase (NRPS) genes (Marahiel et al., 1997) (FIG. 1). mcyA contains two putative adenylation and thiolation domains, a condensation, an N-methyltransferase, and an epimerization domain. In mcyB there are two modules, both include condensation, adenylation, and thiolation domains. mcyC is composed of one module, containing a condensation, an adenylation, a thiolation, and a thioesterase domain (FIG. 1).

Identification of the Polyketide Synthase Genes

The second operon contains six ORFs named mcyG-mcyD-mcyJ-mcyE-mcyF-mcyL A suggested translation start codon (ATG) of mcyG is located 8 bp downstream of a probable RBS (ACAGGA) giving an ORF (7827 bp), which could code for a protein of 2609 amino acids with a predicted mass of 289,859 Da. Another possible initiation is at an ATG, 75 bp upstream from the previously proposed start and 5 bp after a putative RBS (AAGGCA). This ORF. (7905 bp) possibly encodes a protein of 2635 amino acids, 292,851 Da. The ORFs mcyG and mcyD are separated by 96 bp. The translation of mcyD starts probably at an ATG codon 6 bp after a potential RBS (GGAAGGAG), consequently the size of this large ORF is 11,607 bp, encoding 3869 amino acids. Following the stop codon TAG of mcyJ there are 36 bp prior to a presumed ATG initiation codon of mcyE, which is preceded (5 bp) by a possible RBS (GCGGACAA). An alternative ATG start codon for mcyE is 57 bp downstream from the previously proposed one and 3 bp from a possible RBS (AATGGAGG). The two versions (10,446 bp and 10,386 bp) of this large ORF, mcyE, could code for polypeptides of 3482 amino acids, 388,755 Da and 3462 amino acids, 386,501 Da, respectively. The ORF mcyD encodes a polypeptide of 3869 amino acids with the predicted mass of 430,216 Da. mcyD was identified as a polyketide synthase (PKS) gene, whereas mcyG and mcyE have a combined NRPS/PKS gene structure (FIG. 1).

The Additional Genes

We suggest that the ORF mcyJ is initiated with a GTG codon 59 bp downstream of the stop codon (TAA) of mcyD, and 5 bp from a putative Shine-Dalgarno sequence AGGAGAG. There is no ATG codon located nearby. Accordingly, mcyJ is predicted to be 930 bp in length.

A small ORF, mcyF, (756 bp), following mcyE, begins with an ATG codon 42 bp after the previous stop codon TAG and 6 bp from a putative RBS (GGAGAA). The distance between mcyF and the next ORF, mcyI, (1011 bp) is 54 bp, and an alleged RBS (AAGGTTAA) is found 6 bp upstream from the designated start codon ATG of mcyI. Downstream (295 bp) from the stop codon (TAA) of mcyI an ORF, mcyH, (1776 bp) was found. It presumably is initiated from the ATG codon 6 bp after a potential RBS (AAGATG). Another possible translation start codon (ATG) is found 159 bp downstream from the former one and 4 bp from a putative RBS (AGGCATGG). The sizes of these potential McyH polypeptides of 592 and 539 amino acids are 67,731 Da and 61,754 Da, respectively. mcyJ, mcyF and mcyI encode polypeptides of 310,252 and 337 amino acids with predicted masses of 35,812, 28,426, and 36,750 Da, respectively. McyF is similar to aspartate racemases, McyJ belongs to methyltransferases, and McyI is related to D-3-phosphoglycerate dehydrogenases. McyH contains a membrane spanning and an ATP-binding domain of ABC transporters. A BLAST search of McyH found 75% identity (in 589 aa) to NosG from Nostoc sp. GSV224 (AF204805) and 39% identity (in 543 aa) to the hypothetical ABC transporter ATP-binding protein SLL0182 of Synechocystis sp. PCC 6803 (Q55774).

Comparison of Microcystin Synthetase Genes

The microcystin synthetase genes were previously sequenced from M. aeruginosa strains PCC7806 (mcyA-mcyJ, Tillett et al., 2000), K-139 (mcyA-mcyI, Nishizawa et al., 2000) and UV027 (mcyA-mcyC, Raps et al., unpublished, accession no. AF458094), and from Planktothrix agardhii CYA126 (Christiansen et al., 2002). When Anabaena 90 sequences were compared to M. aeruginosa sequences, they revealed 65 to 75 (mcyJ 80%) percent identities at the amino acid level and 69 to 75 (mcyJ 79%) percent identities at the nucleotide level (Table 1). The arrangement of the microcystin synthetase genes from mcyD to mcyJ in Anabaena 90 is different from the organization in M. aeruginosa PCC7806, in M. aeruginosa K-139 (known from mcyD to mcyI) and in Planktothrix agardhii CYA126.

TABLE 1
Percentage identity of the microcystin synthetase genes/polypeptides from
Anabaena strain 90 with the genes/polypeptides sequenced from other
cyanobacteria and the mol % G + C of the genes.

| | mcy/Mcy^{a} | | | | | | | | | |
|---|---|---|---|---|---|---|---|---|---|---|
| | A | B | C | D | E | F | G | H | I | J |
| M. aeruginosa PCC7806 | 69/68 | 72/69 | 74/73 | 72/69 | 75/74 | 71/65 | 74/71 | 74/70 | 74/71 | 79/80 |
| mol % G + C | 41 | 39 | 37 | 40 | 39 | 38 | 38 | 35 | 40 | 39 |
| M. aeruginosa K-139 | 69/68 | 71/69 | 74/73 | 72/69 | 75/75 | 71/65 | 74/71 | 74/70 | 74/72 | |
| mol % G + C | 41 | 39 | 37 | 40 | 39 | 37 | 38 | 36 | 39 | |
| M. aeruginosa UV027 | 69/68 | 73/71 | 74/73 | | | | | | | |
| mol % G + C | 41 | 39 | 37 | | | | | | | |
| P. agardhii CYA126/8 | 67/66 | 72/70 | 80/79 | 77/73 | 78/77 | | 77/74 | 78/75 | | 81/82 |
| mol % G + C | 45 | 39 | 35 | 38 | 38 | | 38 | 35 | | 37 |
| Anabaena 90 mol % G + C | 41 | 38 | 37 | 40 | 38 | 34 | 39 | 36 | 38 | 39 |

^{a}References for the sequences: Microcystis aeruginosa PCC7806, Tillett et al., 2000; M. aeruginosa K-139, Nishizawa, et al., 2000; M. aeruginosa UV027, Raps et al., unpublished, AF458094; Planktothrix agardhii CYA126/8, Christiansen et al., 2003.

When the microcystin synthetase genes were compared to the anabaenopeptilide synthetase genes of Anabaena 90, the highest similarity, 54%, was between mcyC and apdD.

In the genome databases of Anabaena 7120 (http://www.kazusa.or.jp/cyano/Anabaena/search.html) and Nostoc punctiforme (http://www.igi.doe.gov/JGI_microbial/html/nostoc/nostoc_homeoage.html) no genes were found with more than 50% identity to the microcystin synthetase genes at the amino acid level. There are two sequences in the genome database of Anabaena/Nostoc 7120 named “microcystin synthetase B” on account of similarity to mcyB of Microcystis aeruginosa (AY034602): all2643 (ID:3312, 3309 bp) and a112647 (ID:3317, 3261 bp), (identity: 47.0%, positive: 65.5% and identity: 43.9%, positive: 61.9%, respectively). The matches of these sequences with mcyB of Anabaena 90 are 53% and 51% at the gene level. The translated peptides are 49%/66% and 43%/61% identical/similar, respectively.

The G+C content of the microcystin synthetase gene cluster (56 kb) from Anabaena 90 is 39%, is lower than the value, 43%, for the region of the anabaenopeptilide synthetase (39 kb) (Rouhiainen et al., 2000). These figures are in the limits of the mol % G+C values 43.9, 39.1 and 42.3 for the type strains Anabaena cylindrica (PCC 7122), Anabaena flos-aquae (1?CC 9332) and for the reference strain of Anabaena cluster 2 (PCC 7108), respectively (Rippka et al., 2001).

Substrate Specificity of the Adenylation Domains

The substrate specificity-conferring amino acids in the adenylation domains of the microcystin synthetases of Anabaena 90, P. agardhii CYA126, M. aerginosa PCC7806, K-139, and UV027 were assessed according to Stachelhaus et al., (1999) (Table 2). The substrate specificity codes of the modules McyA-1, McyA-2, McyB-2 and of the nonribosomal peptide synthetase (NRPS) modules in McyG and mcyE are identical or nearly identical in all the sequenced microcystin synthetases (able 2).

TABLE 2
Specificity-conferring amino acids (signature sequences) of the adenylation
domains in the microcystin synthetases from different cyanobacterial strains.

| | | Signature | | Activated | Reference |
|---|---|---|---|---|---|
| Module | Strain | sequence^{a} | Precedent SS | amino acid | template |
| McyA | Anabaena 90 | DVWHISLID | DVWHLSLID | Ser | SyrE (1, 2)^{b} |
| | M. aeruginosa 7806 | DVWHFSLID | DVWHFSLVD | | EntF, MycC (1, 23)^{b} |
| | M. aeruginosa K-139 | DVWHFSLID | | | |
| | M. aeruginosa UV027 | DVWHFSLID | | | |
| | P. agardhii CYA 126/8 | DVWHISLID | | | |
| McyA 2 | Anabaena 90 | DLFNNALTY | | | |
| | M. aeruginosa 7806 | DLFNNALTY | | | |
| | M. aeruginosa K-139 | DLFNNALTY | DLFNNALTY | Ala | BlmIX, MxA (4, 5)^{c} |
| | M. aeruginosa UV027 | DLFNNALTY | | | |
| | P. agardhii CYA 126/8 | DLFNNALSY | | | |
| McyB 1 | Anabaena 90 | DVWFFGLVD | | | |
| | M. aeruginosa 7806 | DAWFLGNVV | DAWFLGNVV | Leu | BacA, LicA, LicB, |
| | M. aeruginosa K-139 | DAWFLGNVV | | | SrFA (1)^{b} |
| | M. aeruginosa UV027 | DVWTIGAVE | | (Arg) | |
| | P. agardhii CYA 126/8 | DALFFGLVD | | | |
| MCyB 2 | Anabaena 90 | DARHVGIFV | | | |
| | M. aeruginosa 7806 | DARHVGIFV | | | |
| | M. aeruginosa K-139 | DABHVGIFV | no precedents | (Asp/MeAsp) | |
| | M. aeruginosa UV027 | DARHVGIFV | | | |
| | P. agardhii CYA 126/8 | DPRHVGIFI | | | |
| McyC | Anabaena 90 | DVWCFGLVD | | | |
| | M. aeruginosa 7806 | DVWTIGAVD | | | |
| | M. aeruginosa K-139 | DVWTIGAVE | no precedents | (Arg) | |
| | M. aeruginosa UV027 | DVWTIGAVD | | | |
| | P. agardhii CYA 126/8 | DPWGFGLVD | | | |
| McyG | Anabaena 90 | GAFWVAASG | | | |
| | M. aeruginosa 7806 | GAFWVAASG | no precedents | | |
| | M. aeruginosa K-139 | GAFWVAASG | | | |
| | P. agardhii CYA 126/8 | GAFWVAASG | | | |
| McyE | Anabaena 90 | DPRHSGVVG | | | |
| | M. aeruginosa 7806 | DPRHSGVVG | no precedents | (Glu) | |
| | M. aeruginosa K-139 | DPRHSGVVG | | | |
| | P. agardhii CYA 126/8 | DPRHSGVVG | | | |

^{a}Nine variable amino acids of the signature sequences determined as described by Stachelhaus et al., 1999.
Bold letters indicate the residues, which are identical with the amino acids of the signature sequence from Anabaena 90.
^{b}1. Stachelhaus et al., 1999, 2. Challis et al., 2000, 3. Duitman et al., 1999.
^{c}4. Du et al., 2000, 5. Silakowski et al., 2001.

There are, however, more differences in the specificity codes of variable amino acids activating McyB-1 and McyC module. The substrate specificity regions of the adenylation domains (corresponding amino acids 235-331 of GrsA, Stachelhaus et al., 1999) in McyA, McyB and in McyC from Anabaena 90, P. agardhii and from M aeruginosa were compared by using the algorithm of Smith and Waterman in the EMBOSS program package. The substrate specificity regions of McyA, of the second module of McyB (McyB-2) and of McyC are highly conserved. In Anabaena 90 and M. aeruginosa, the identity/similarity values are 80/90% for McyA, 86/92% for McyB-2 and 70/80% for McyC. Between Anabaena 90 and P. agardhii the identity/similarity for the substrate specificity region of McyC is higher, 85/88%, but lower for the second module of McyA, 73/83%. The substrate specificity region of McyB-1 is considerably less conserved between Anabaena 90 and M. aeruginosa PCC7806, 29/53% than between Anabaena 90 and M aeruginosa UV027, or P. agardhii, 66/80%.

Activities Encoded by mcyG, mcyD and mcyE of Anabaena 90

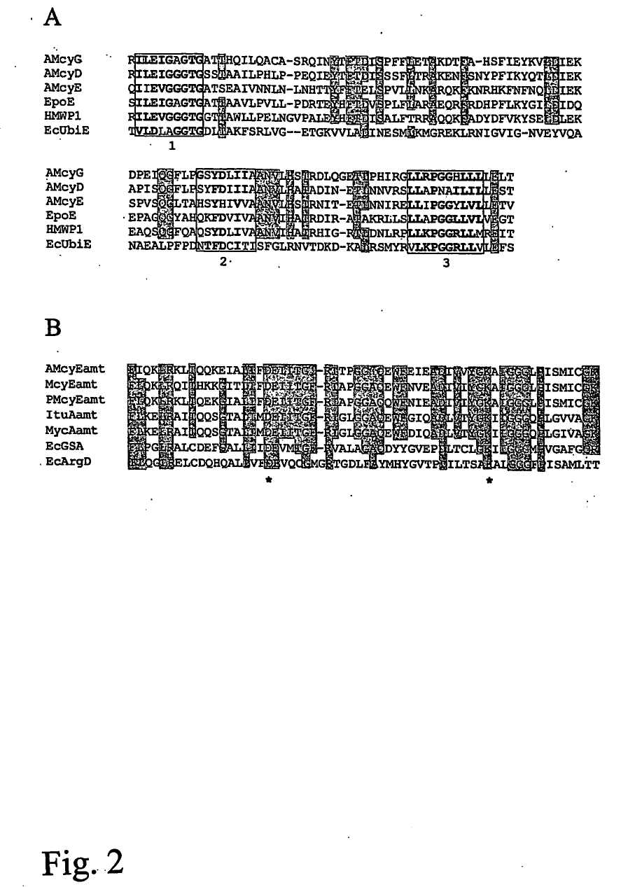

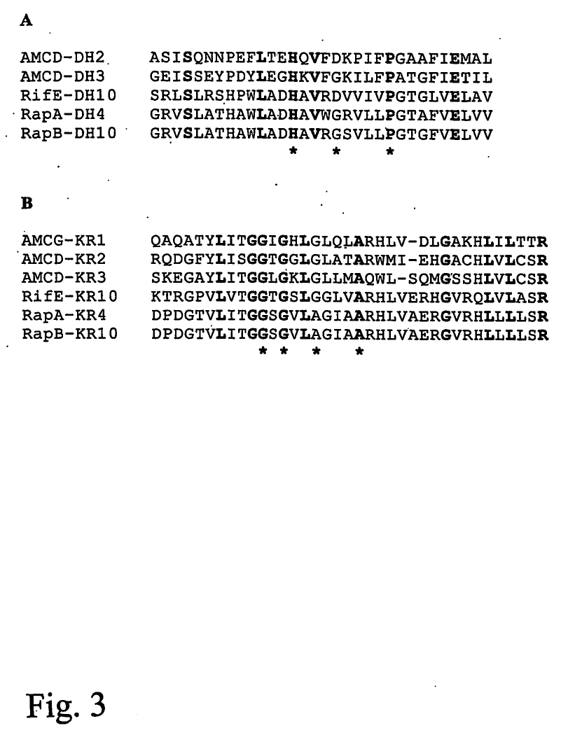

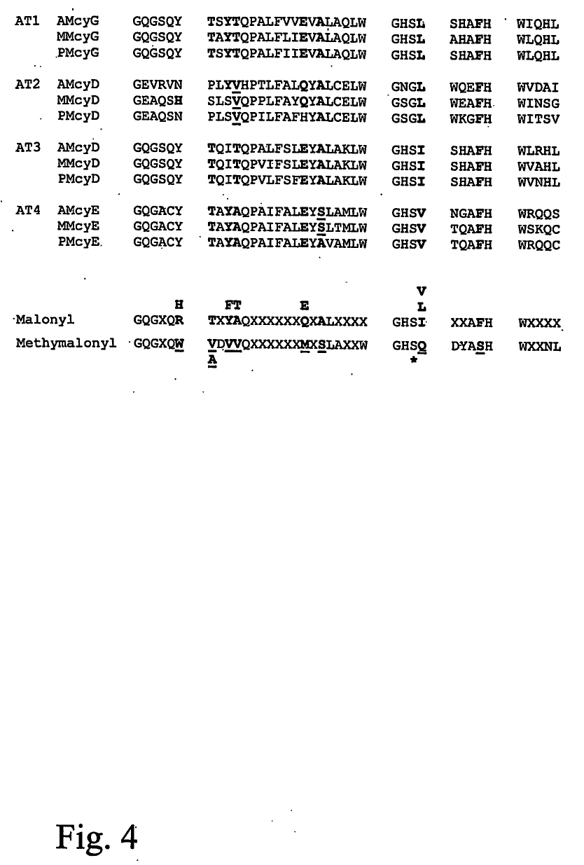

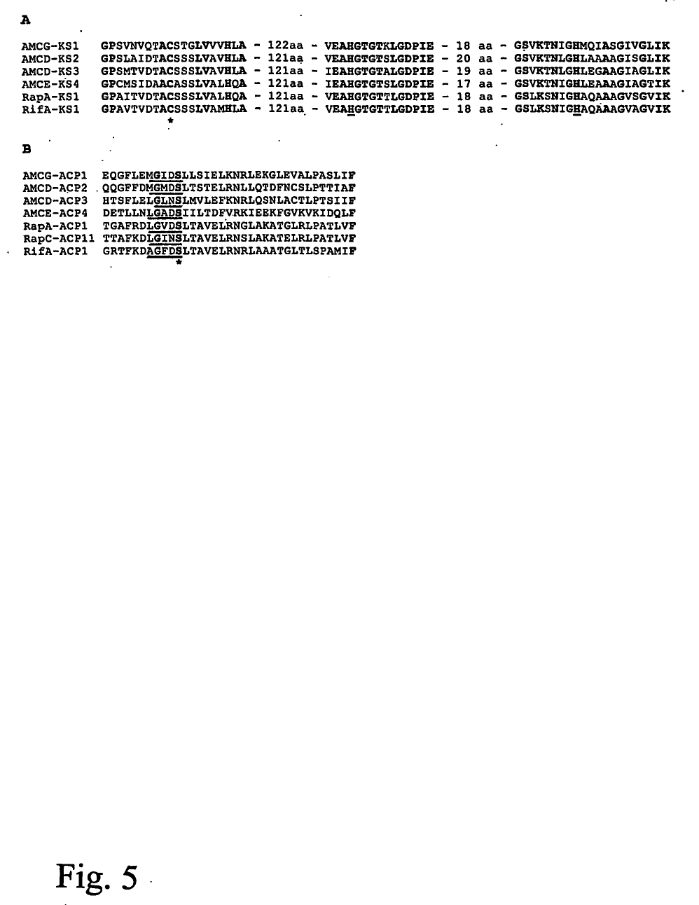

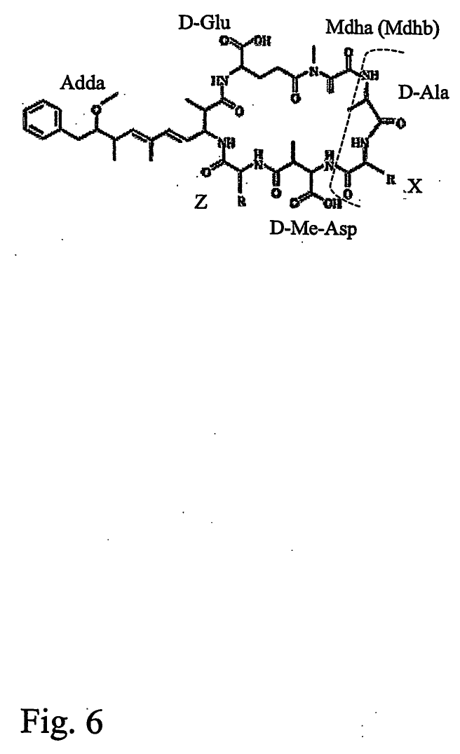

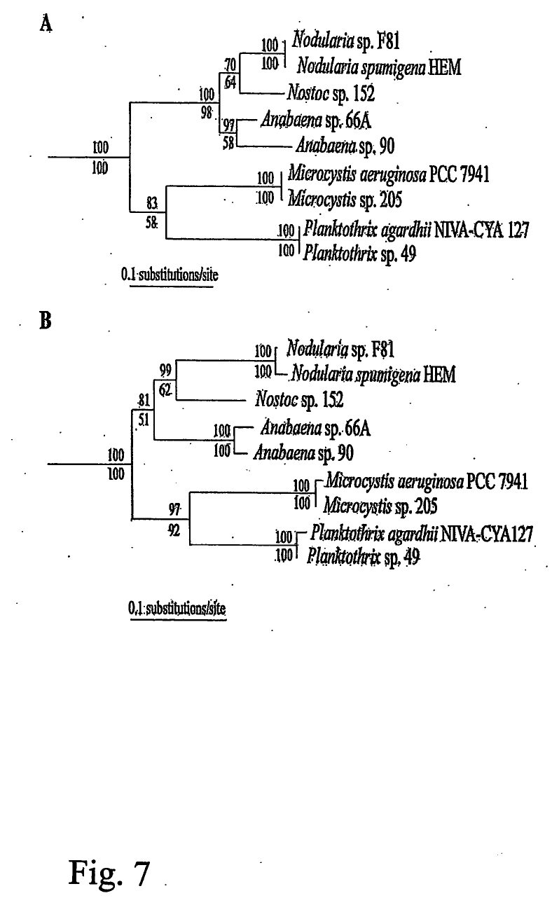

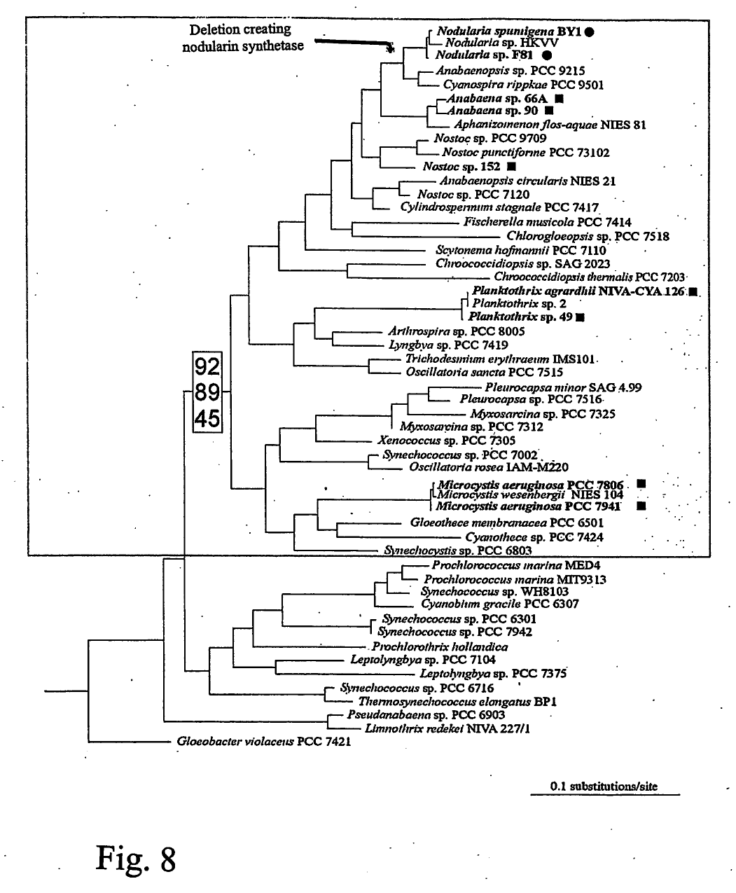

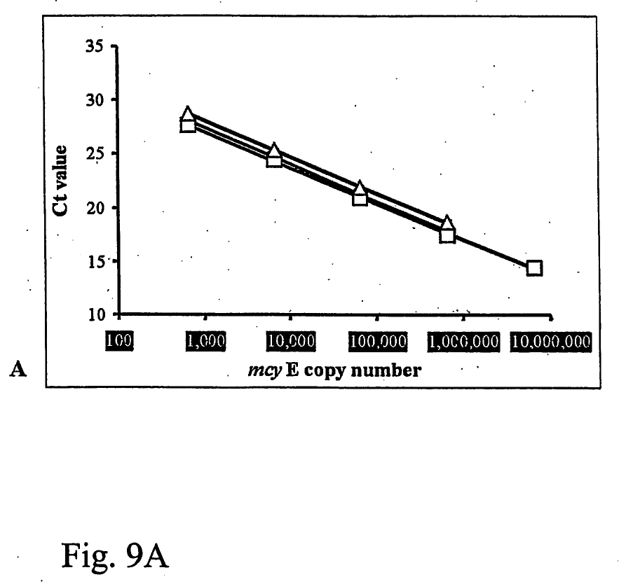

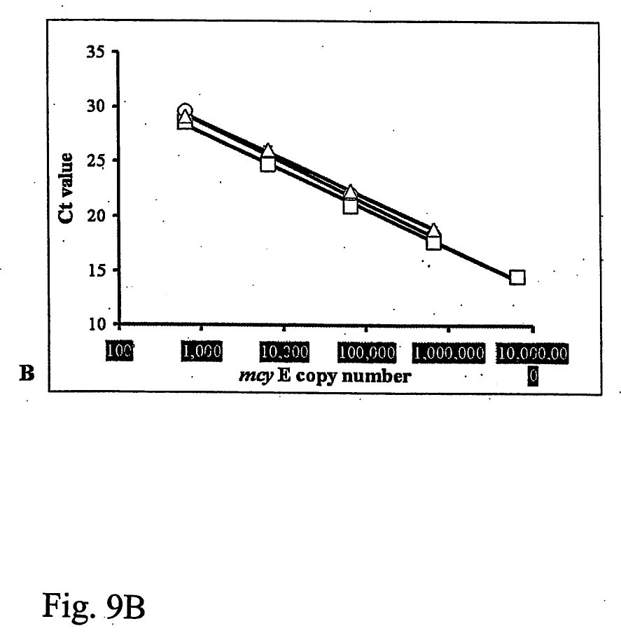

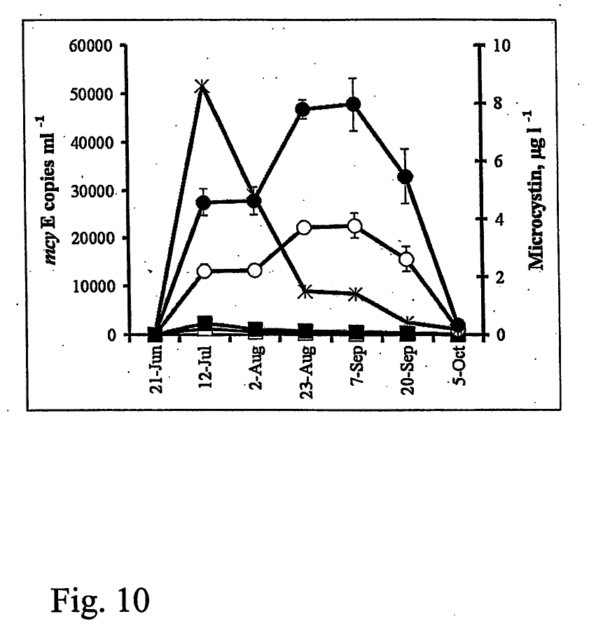

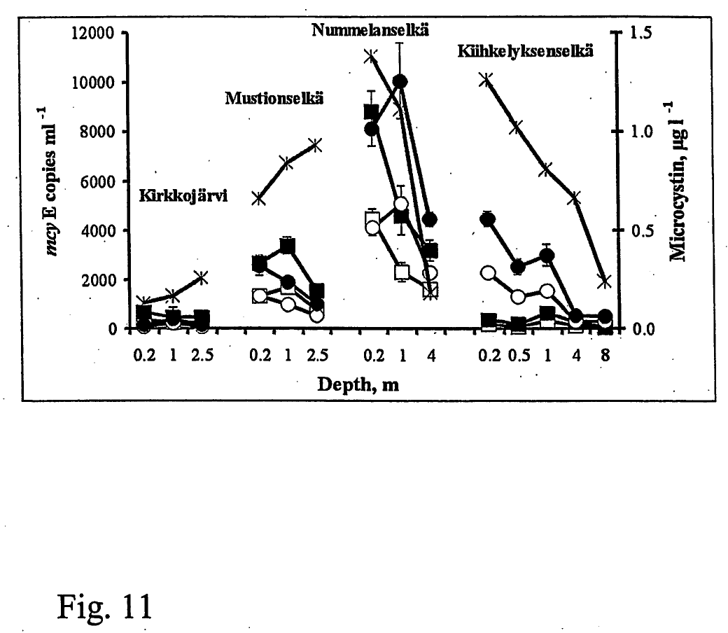

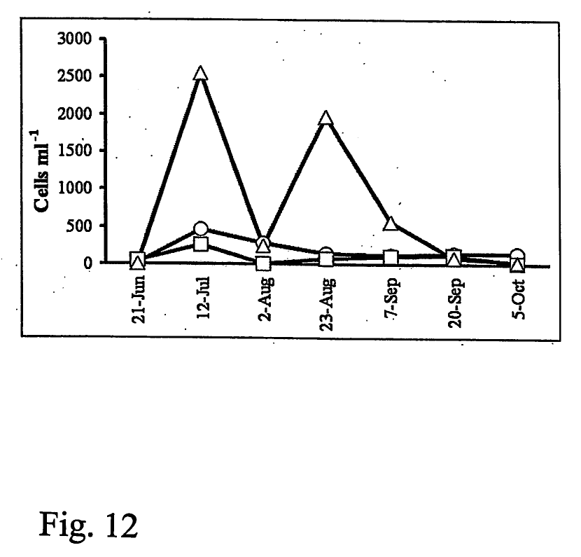

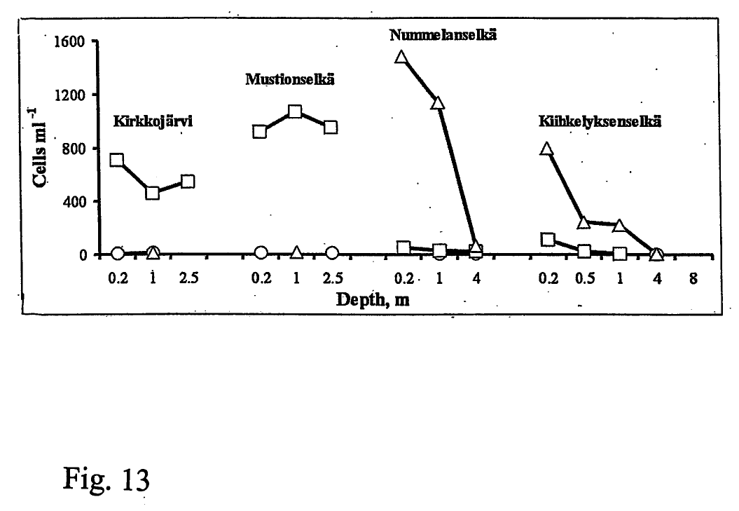

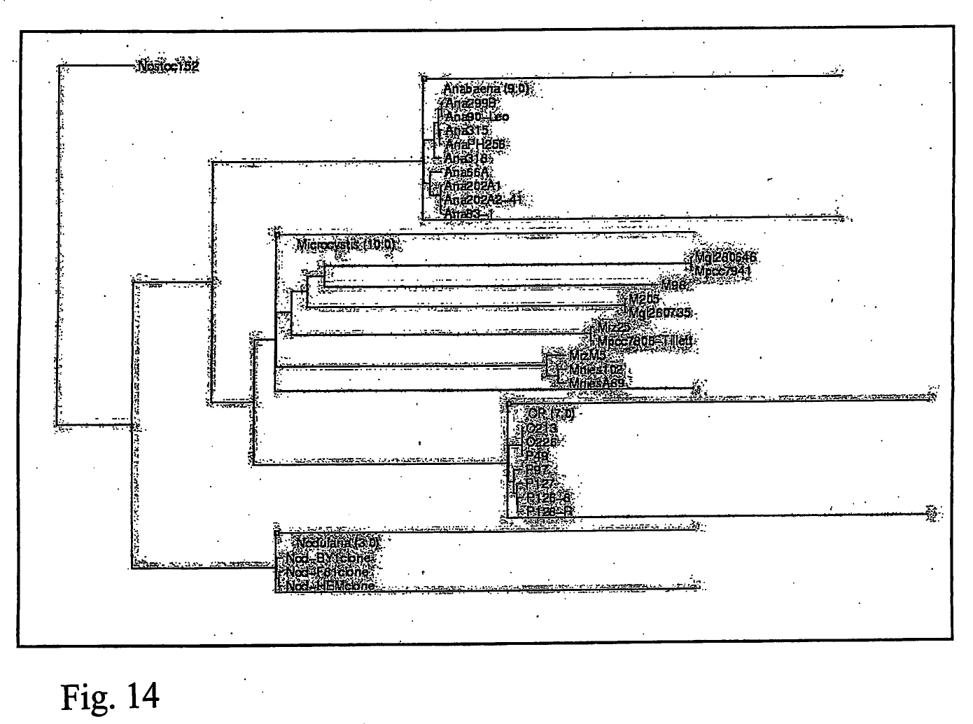

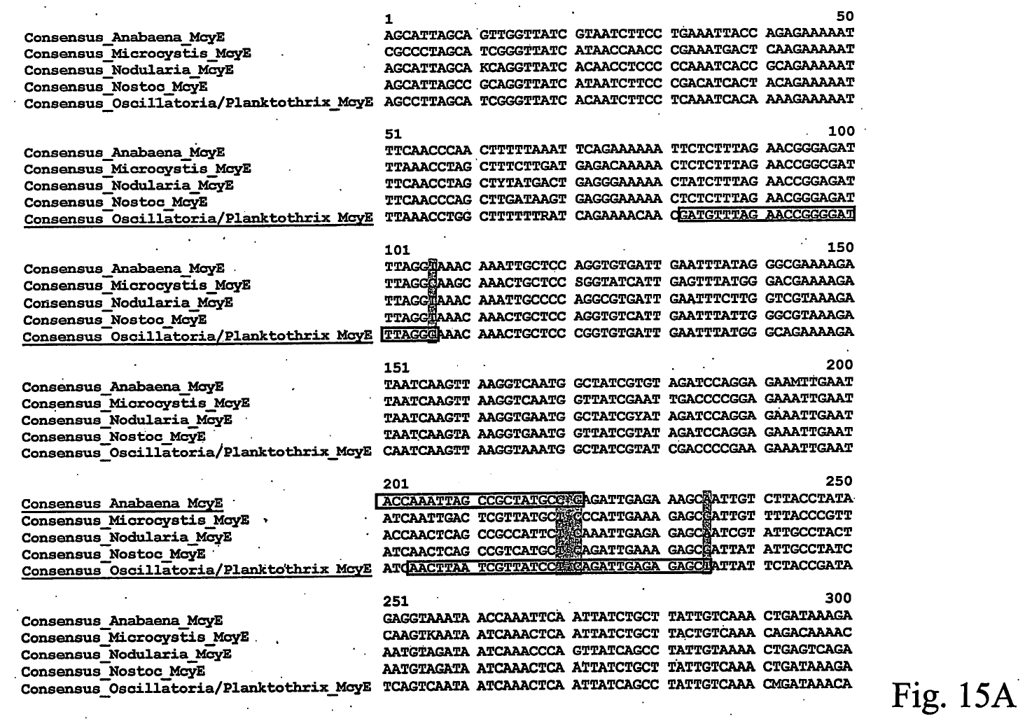

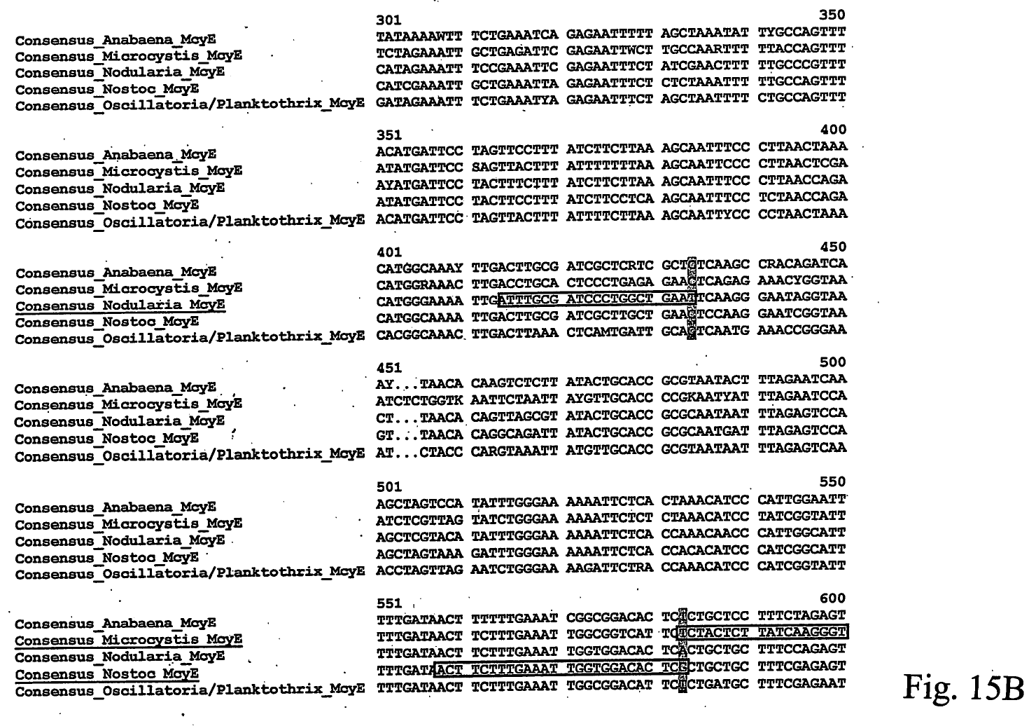

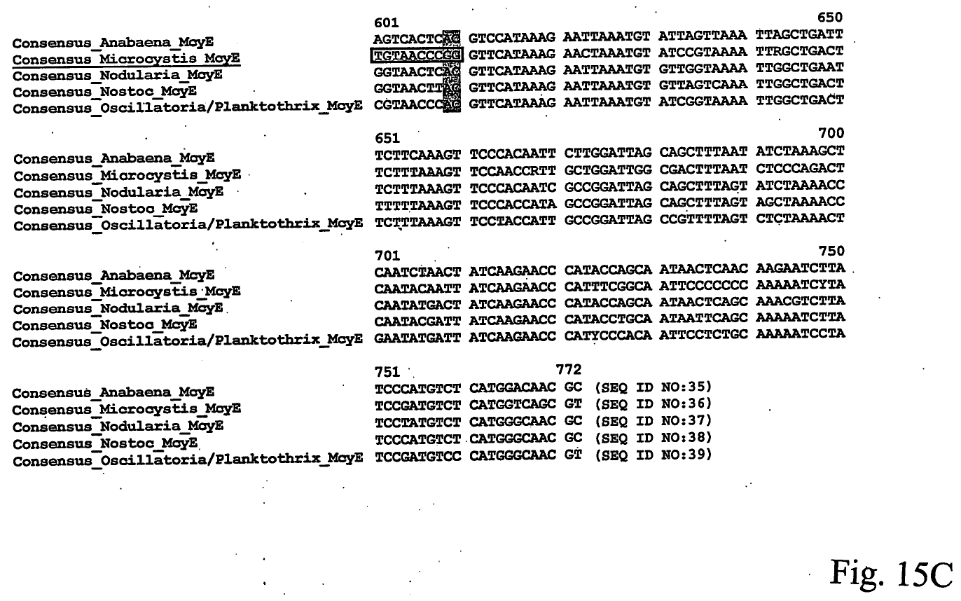

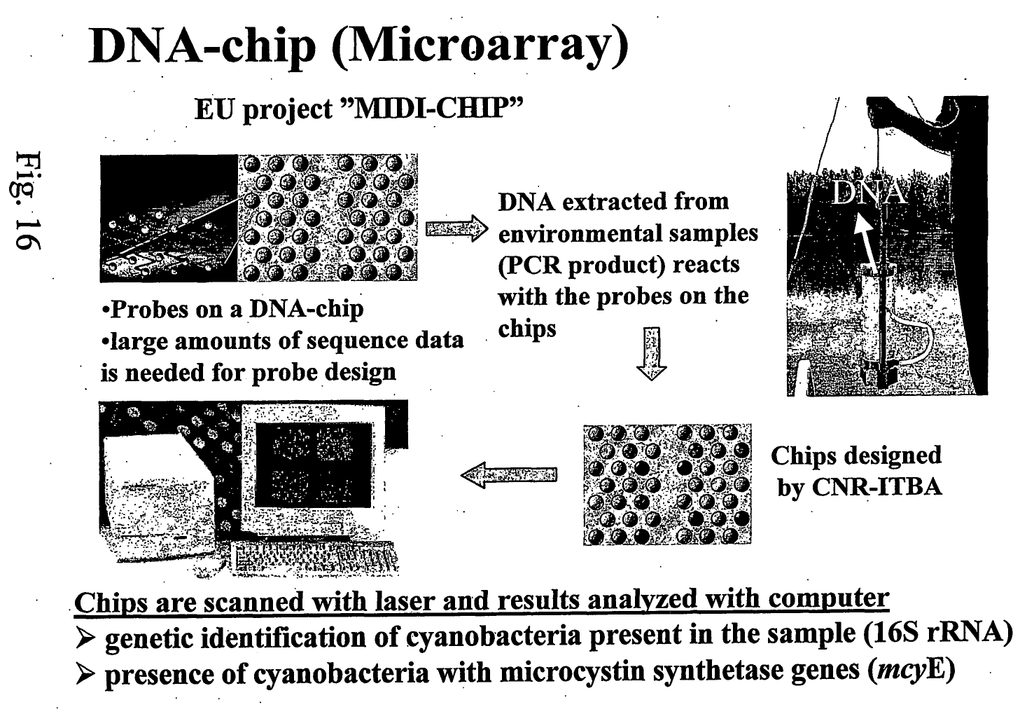

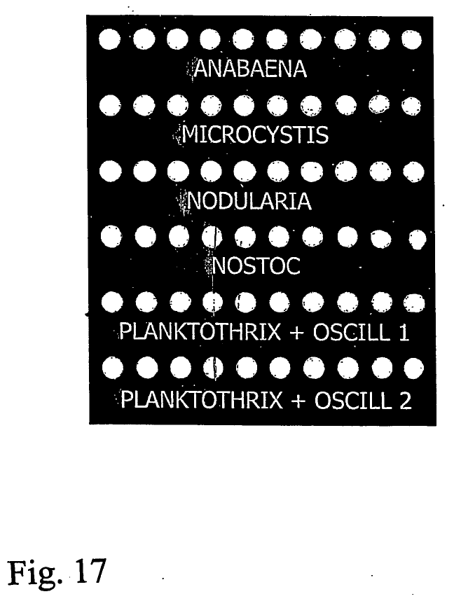

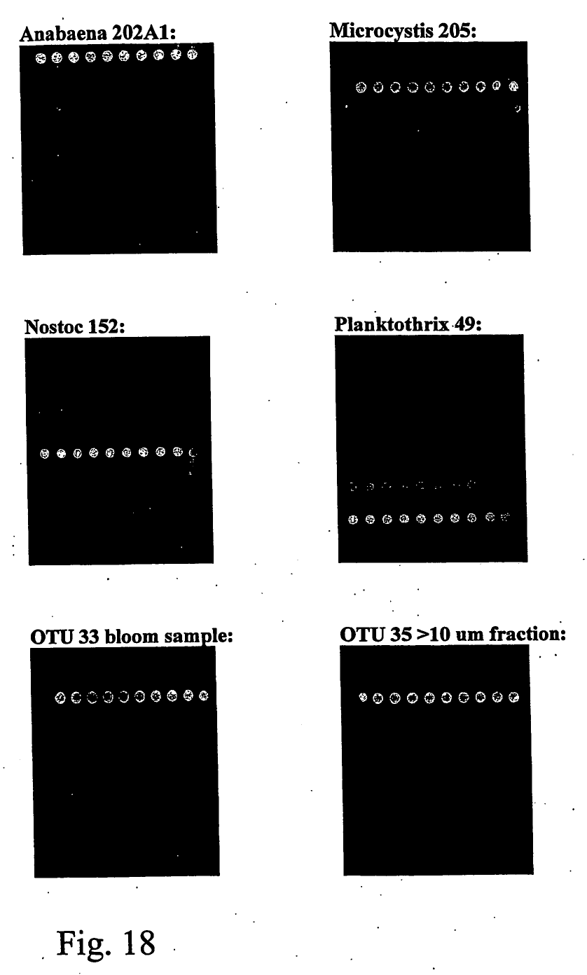

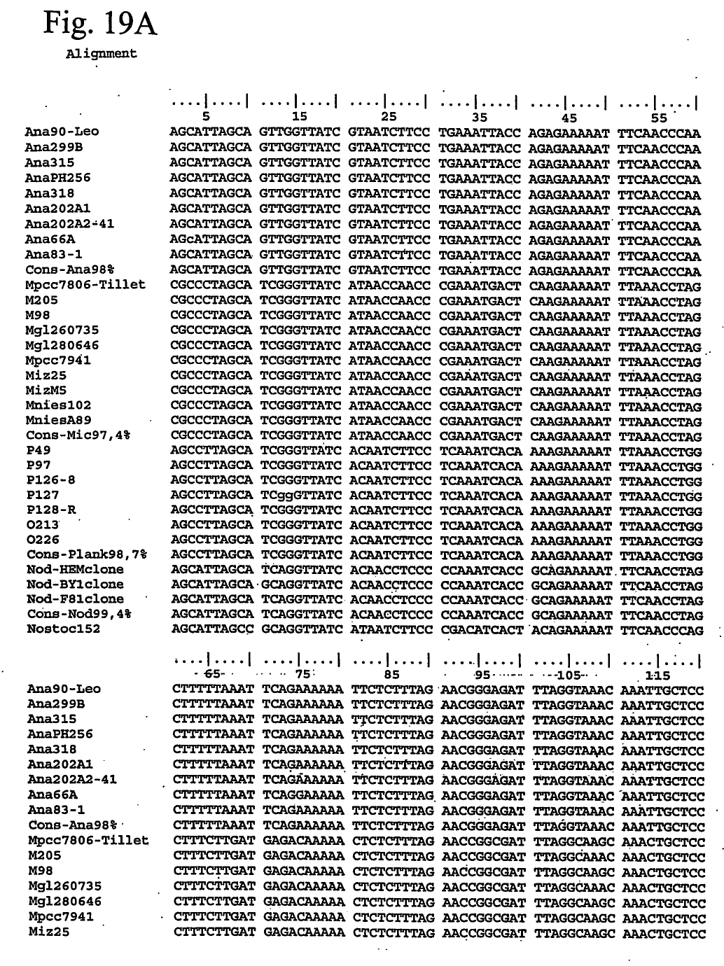

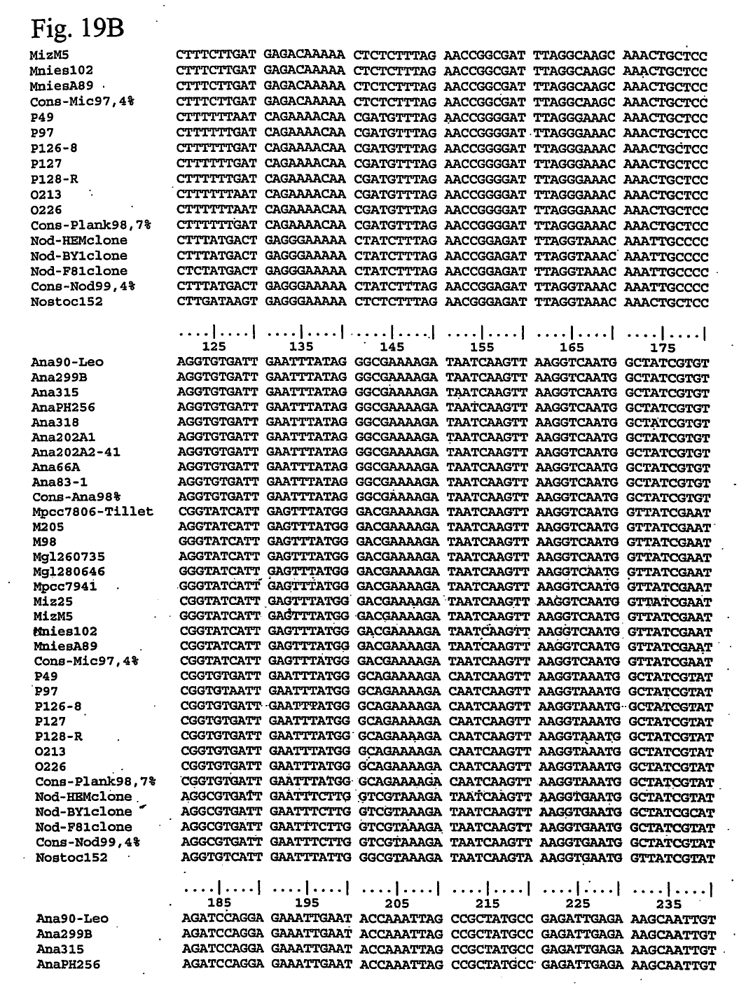

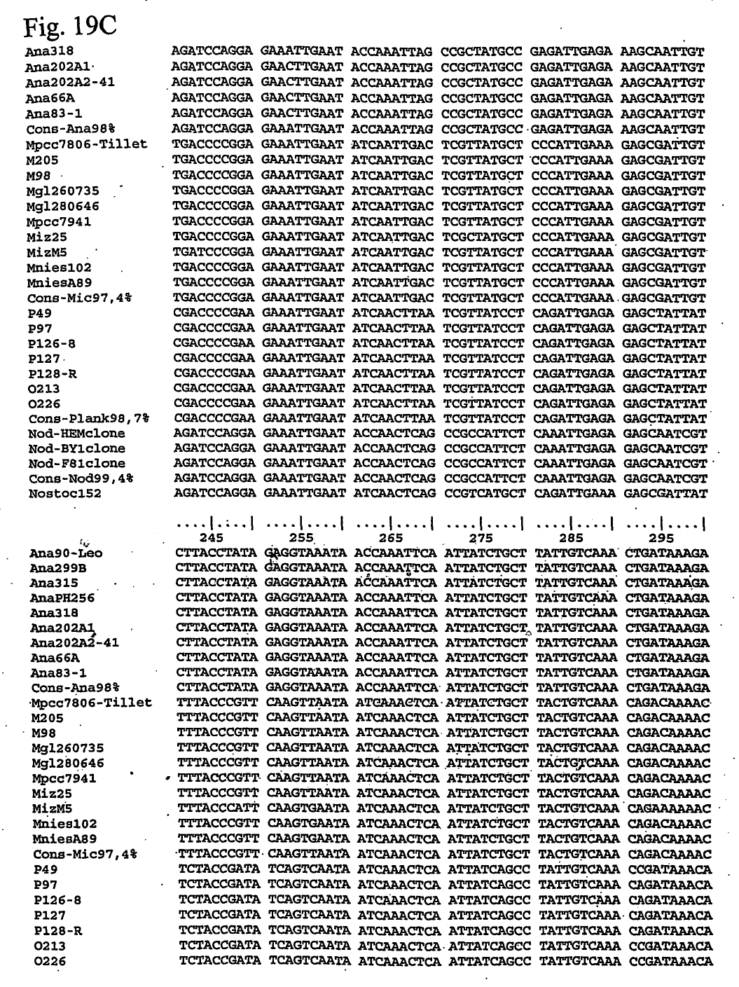

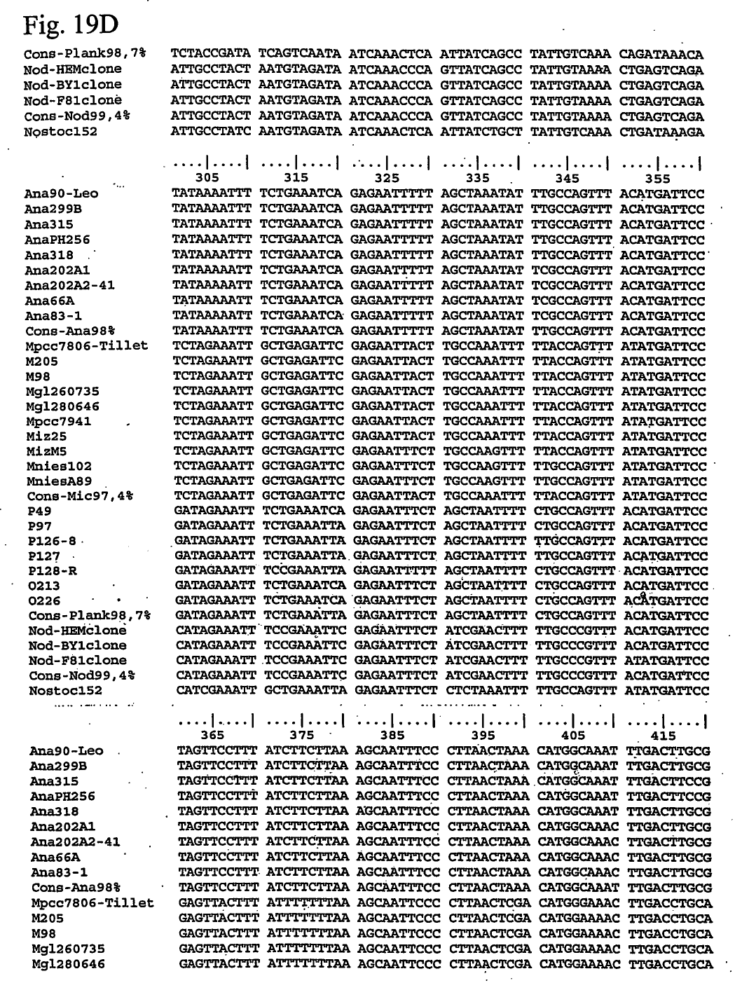

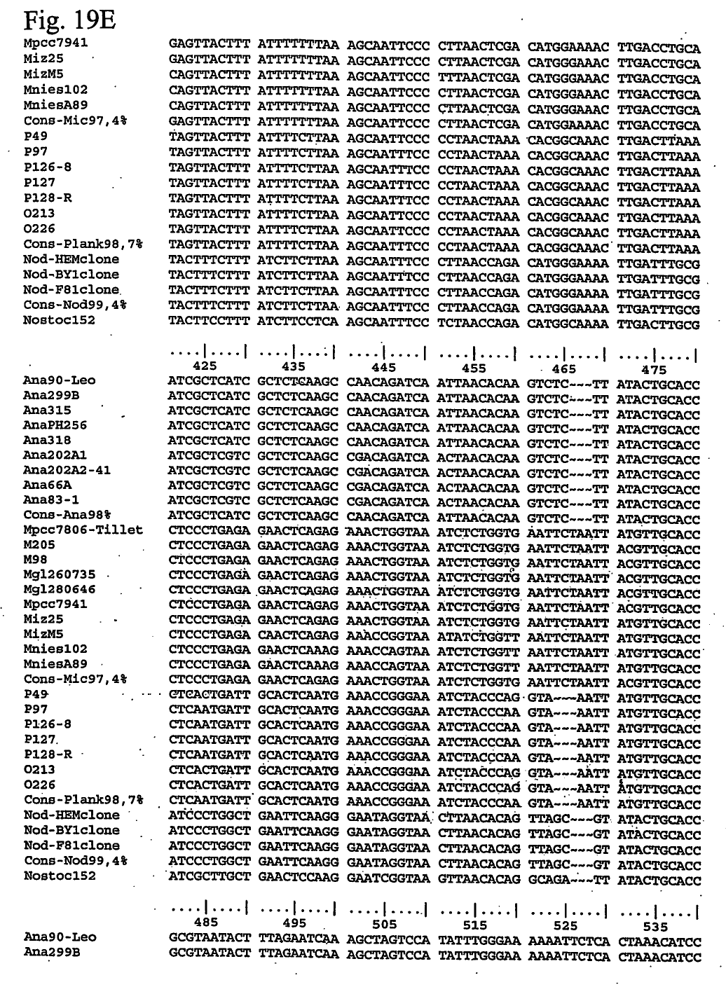

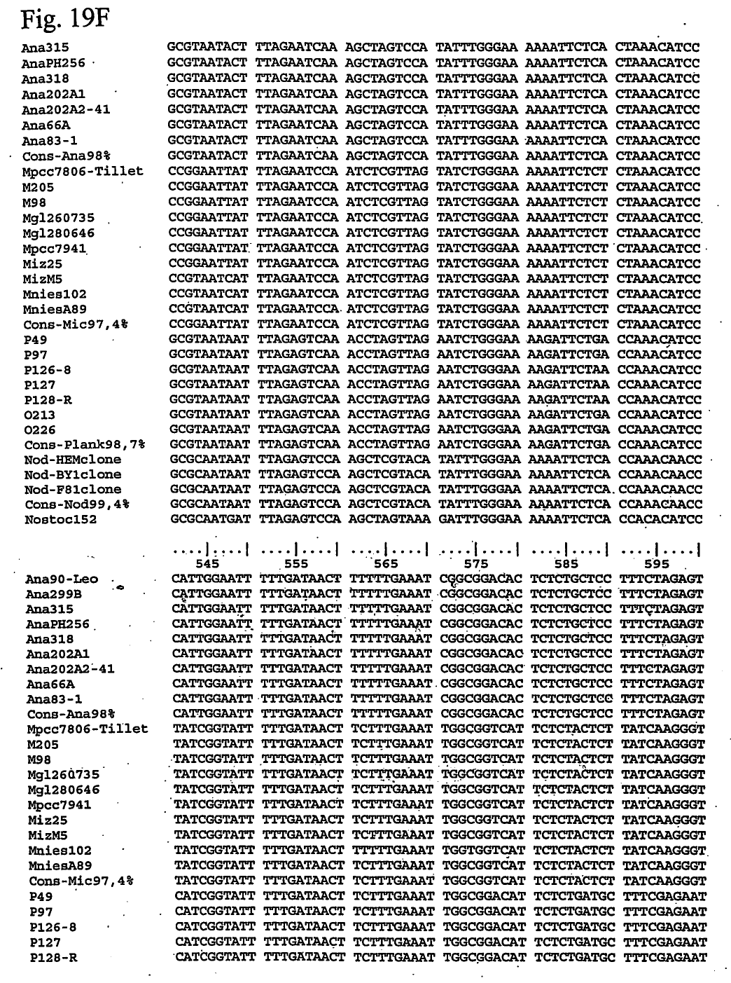

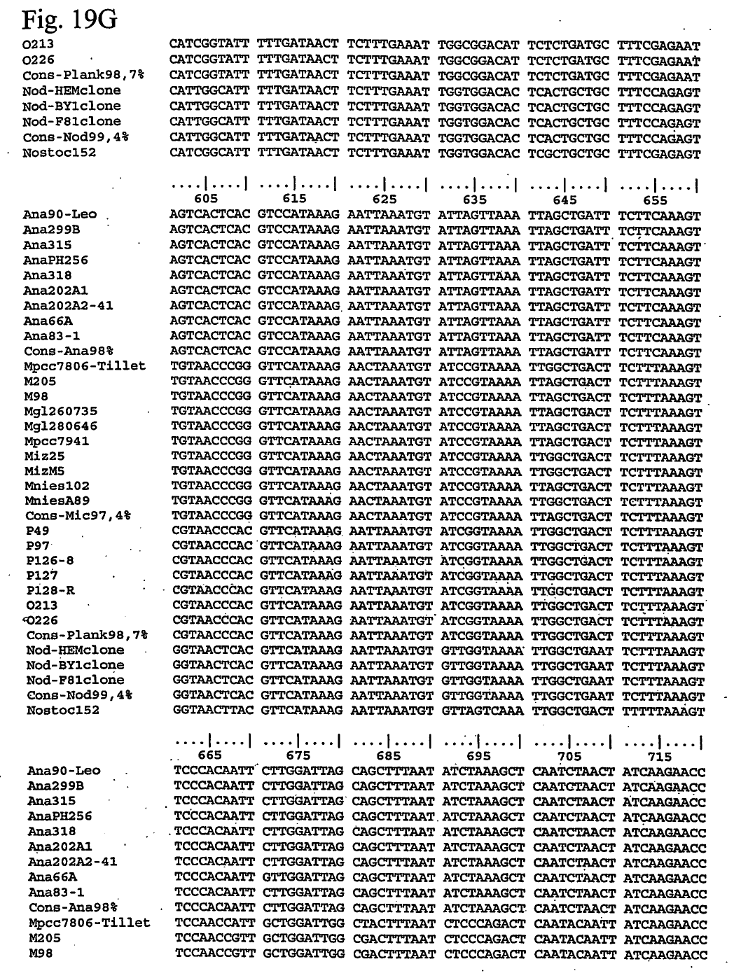

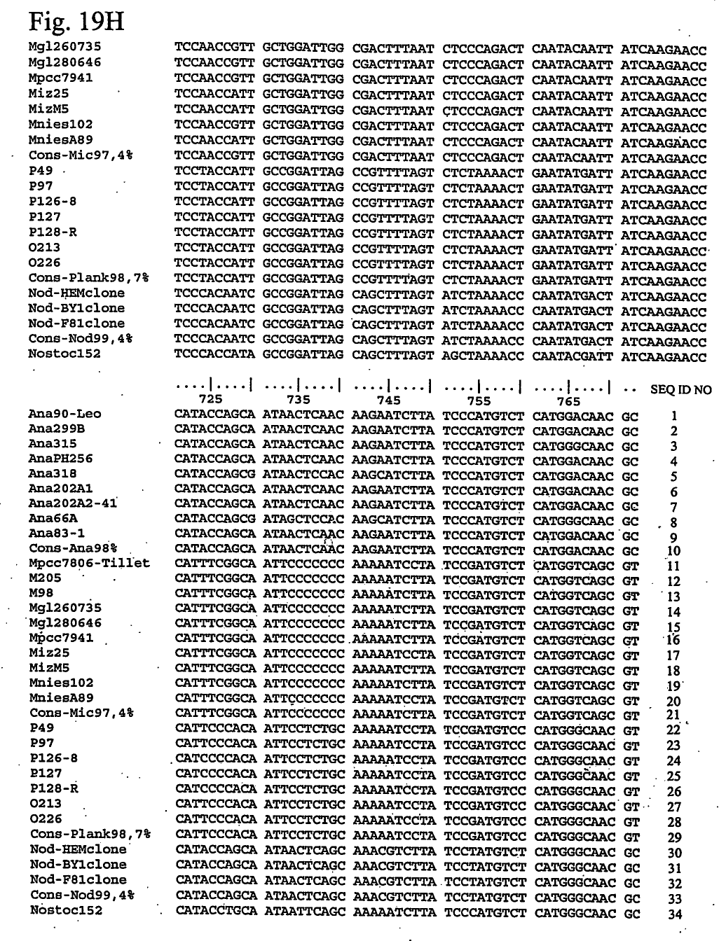

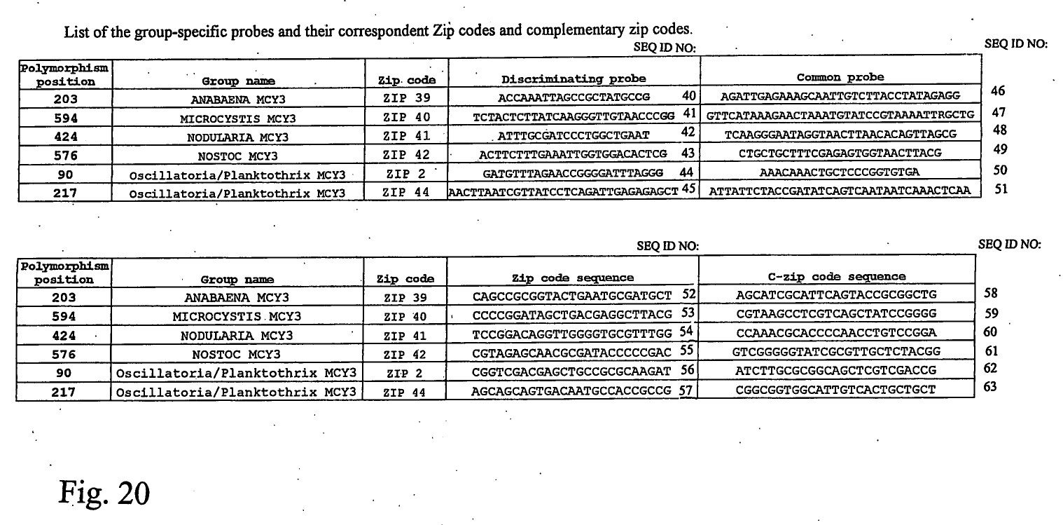

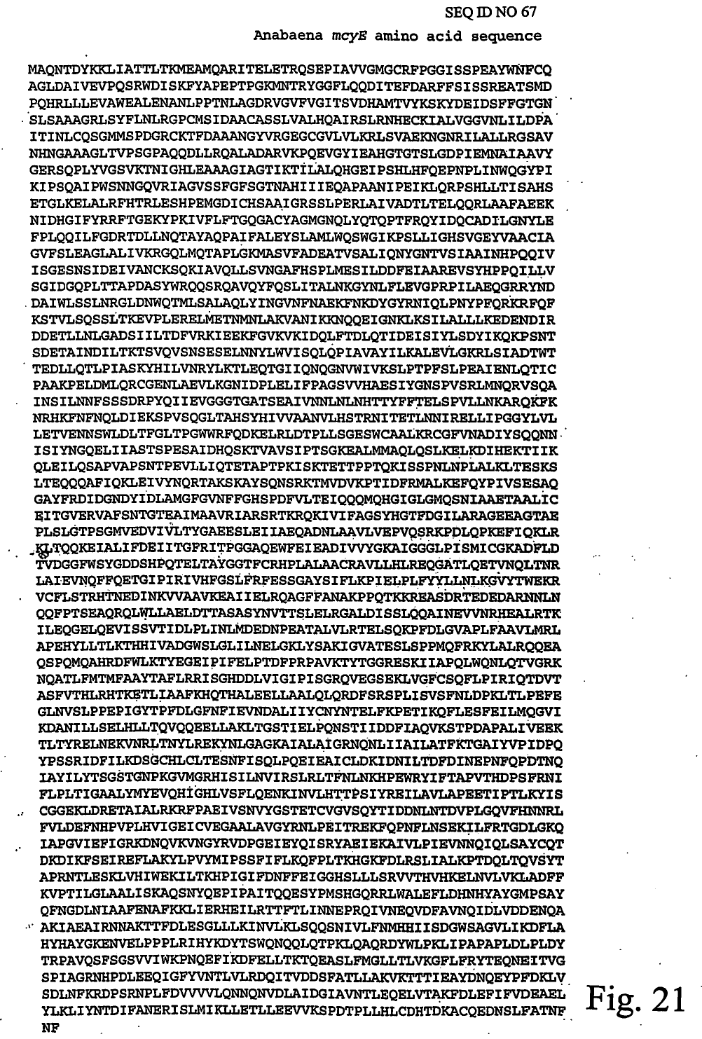

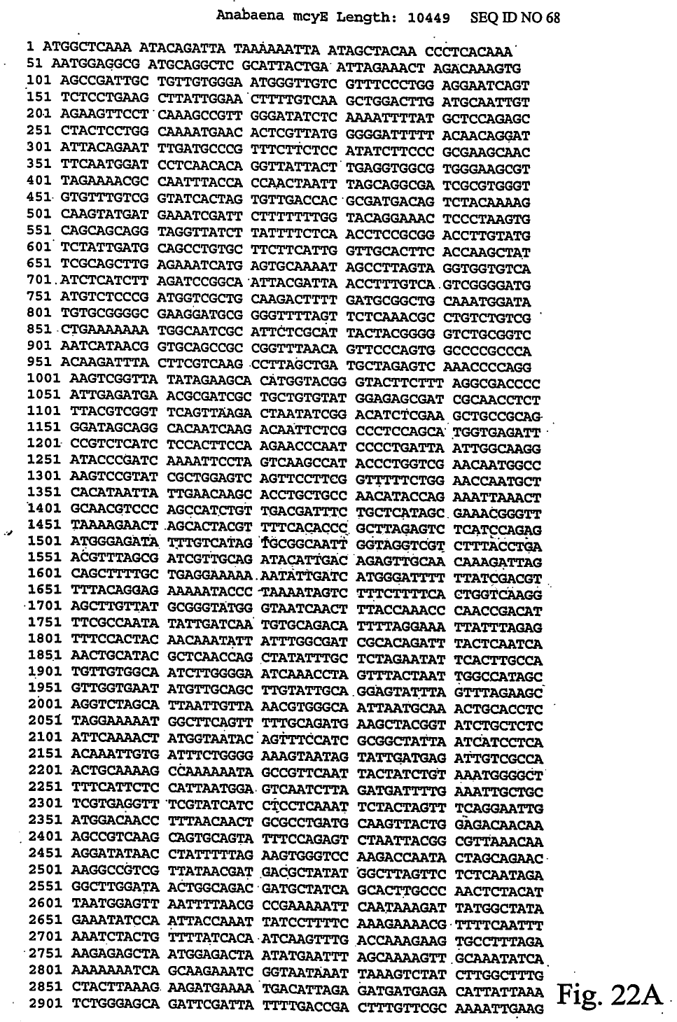

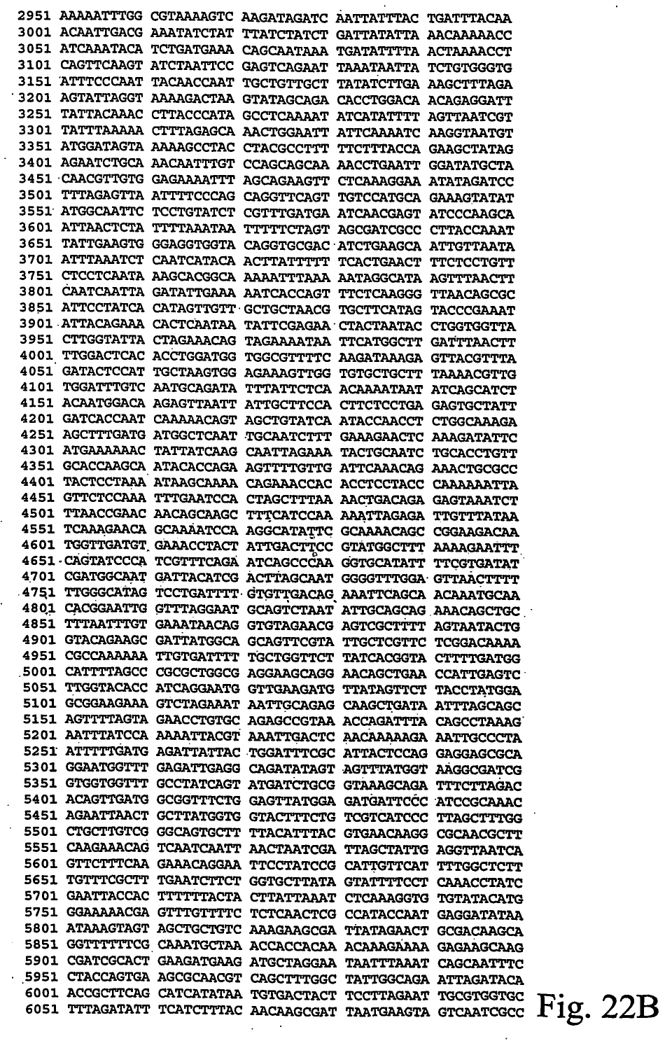

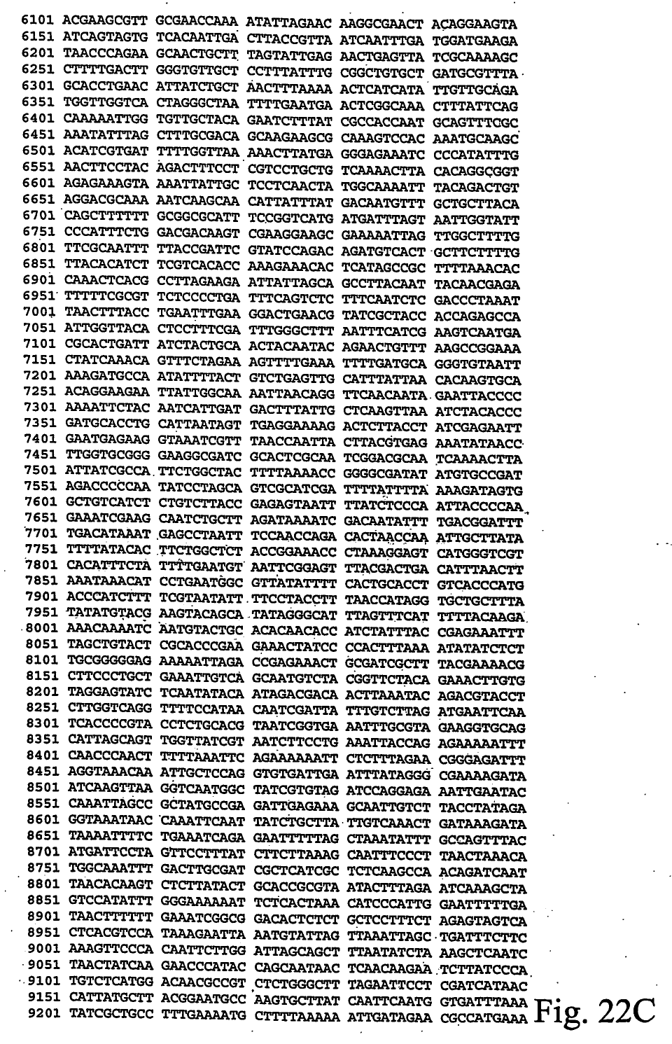

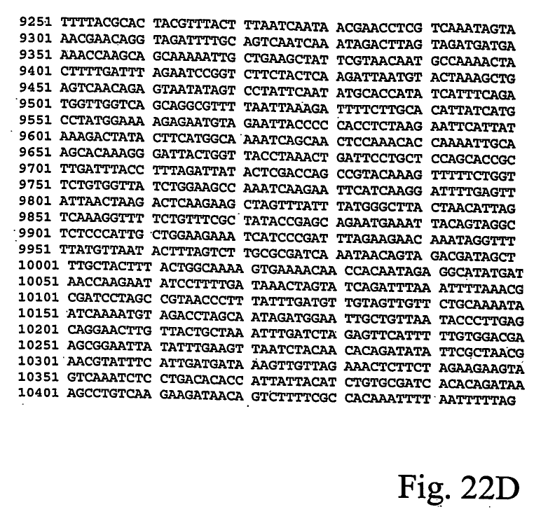

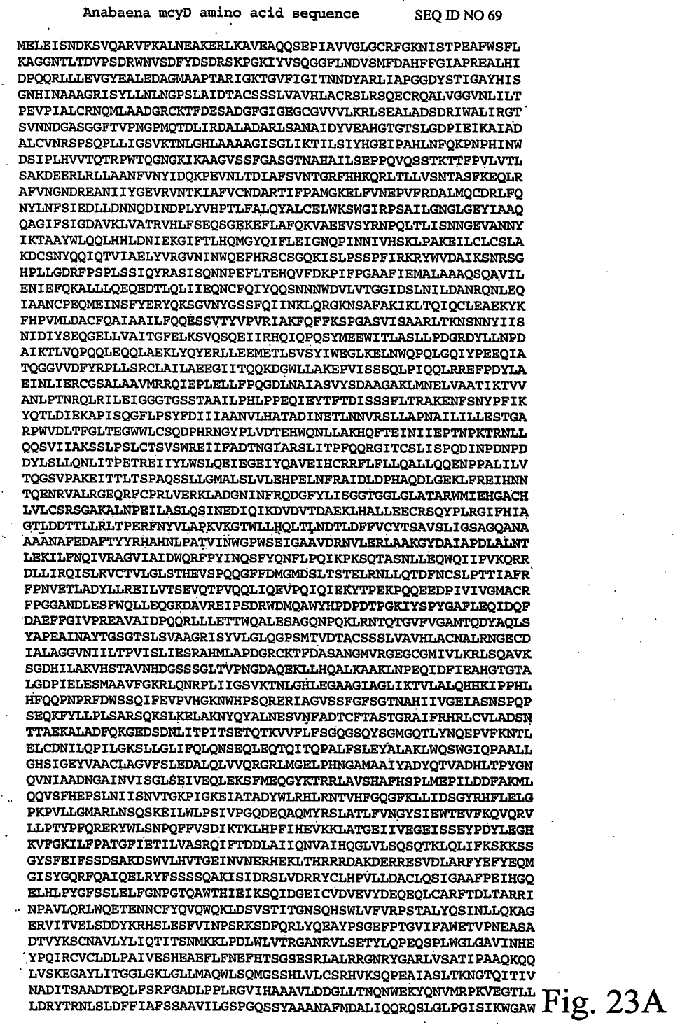

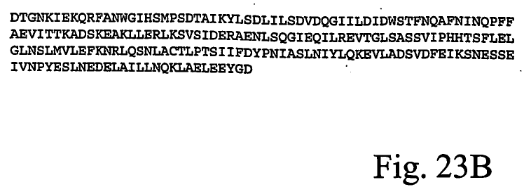

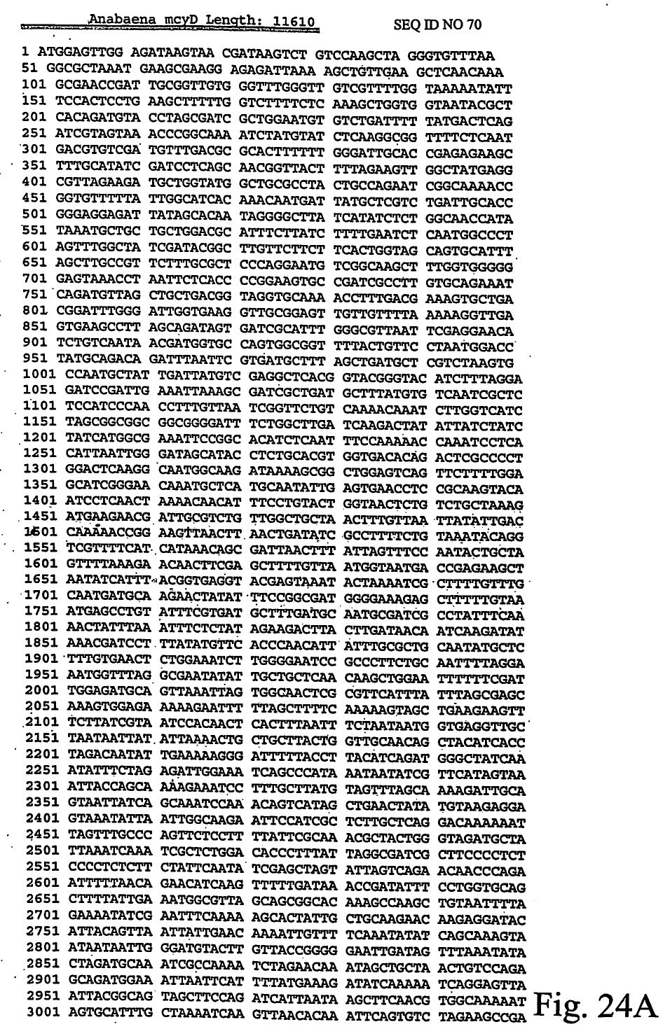

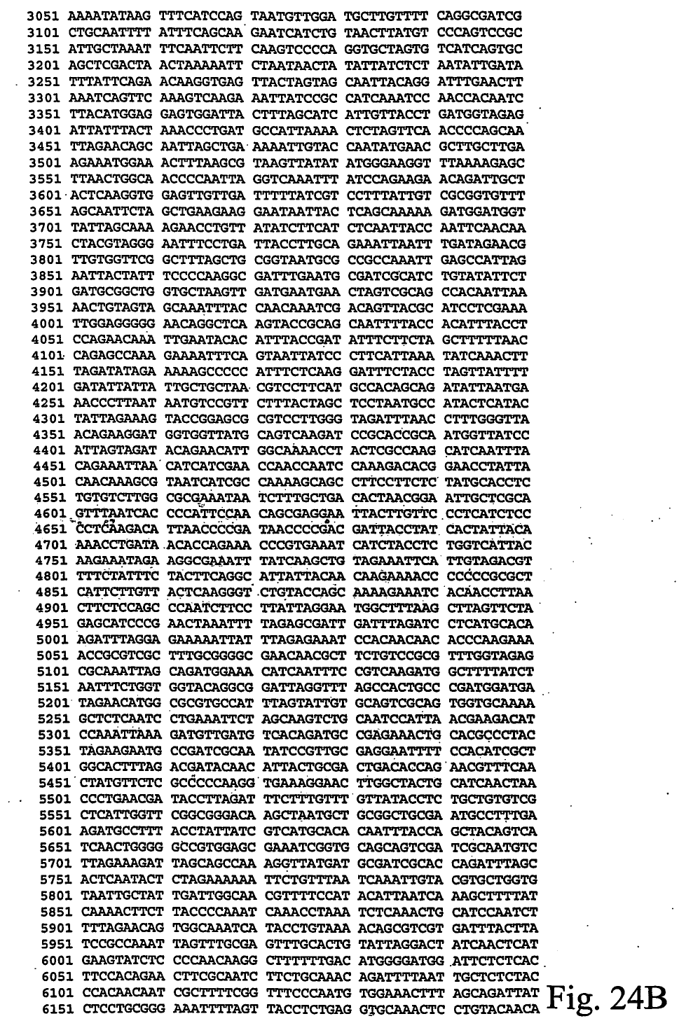

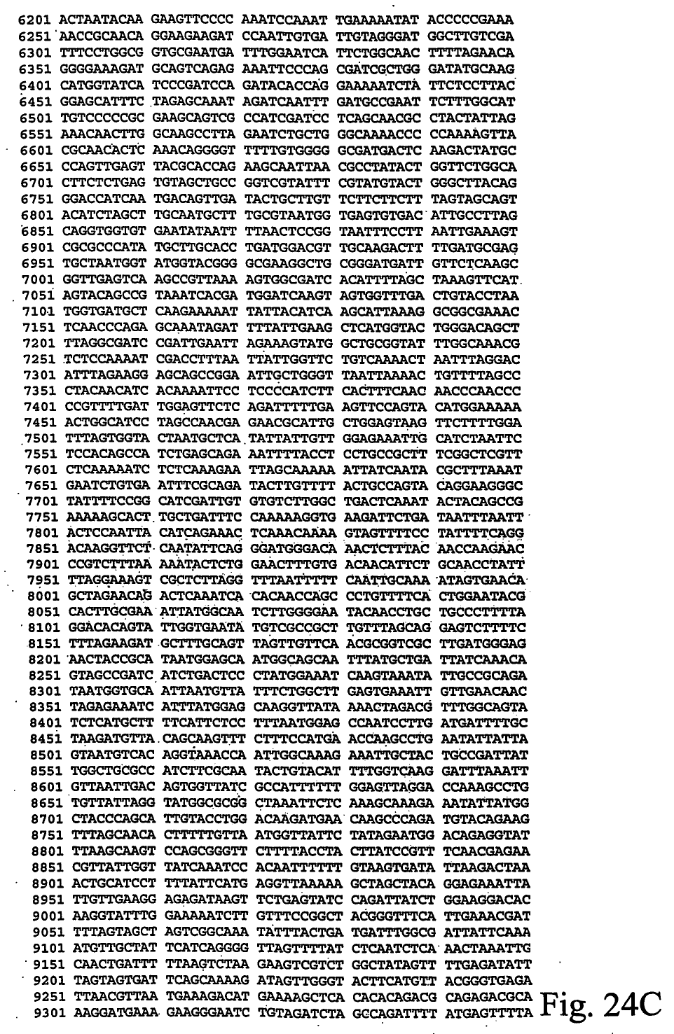

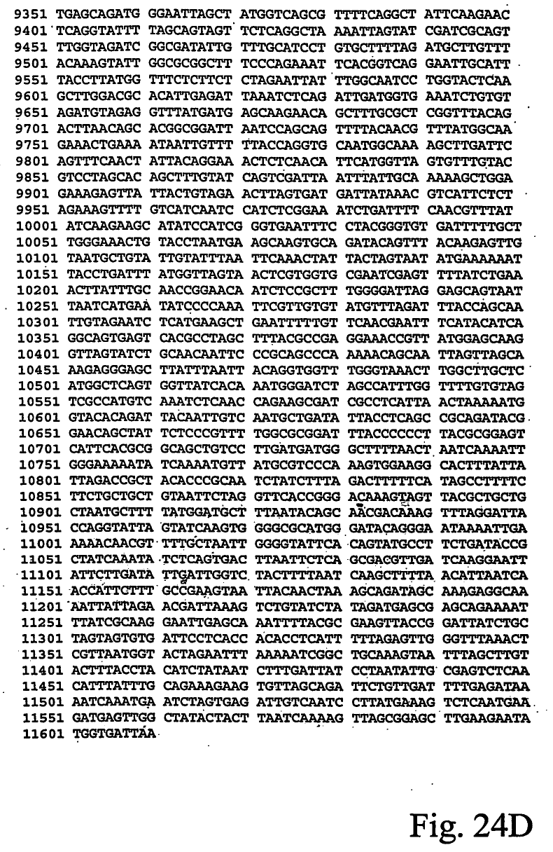

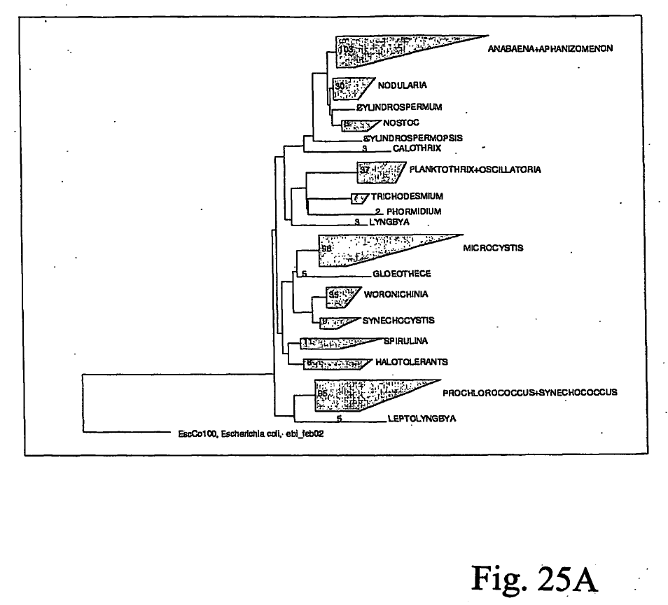

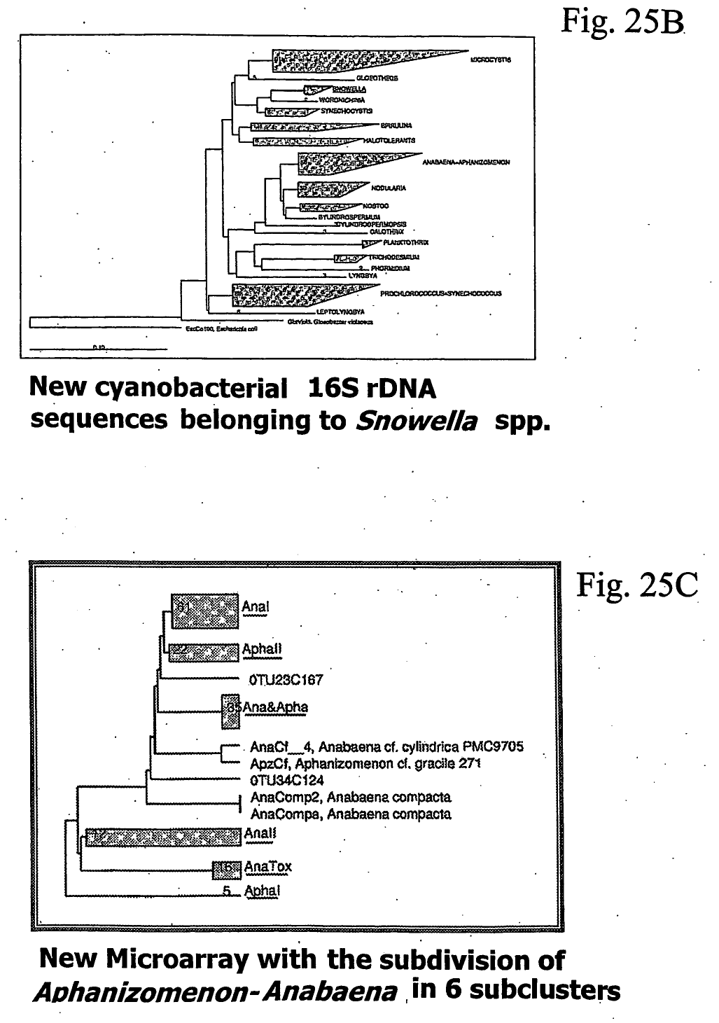

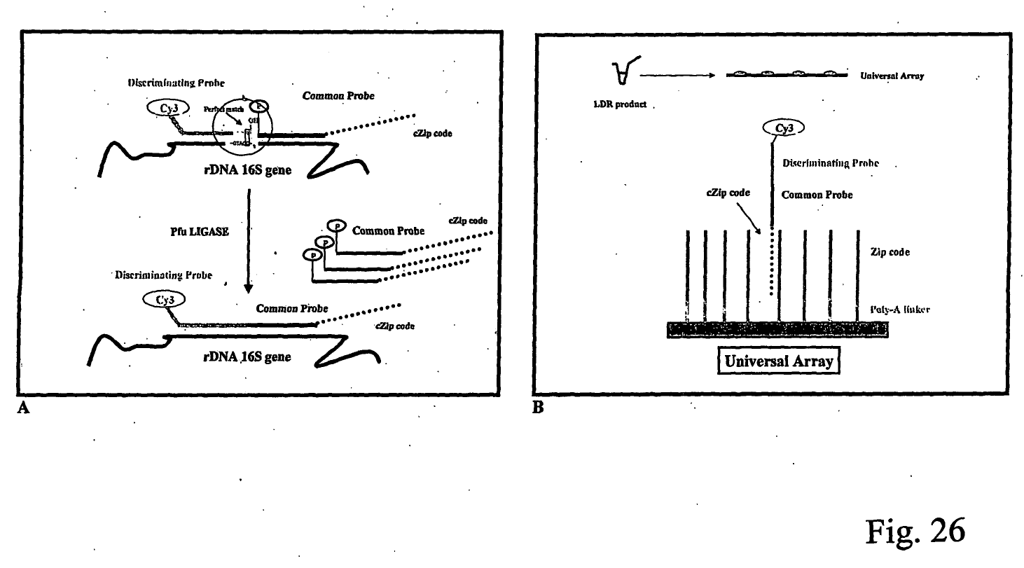

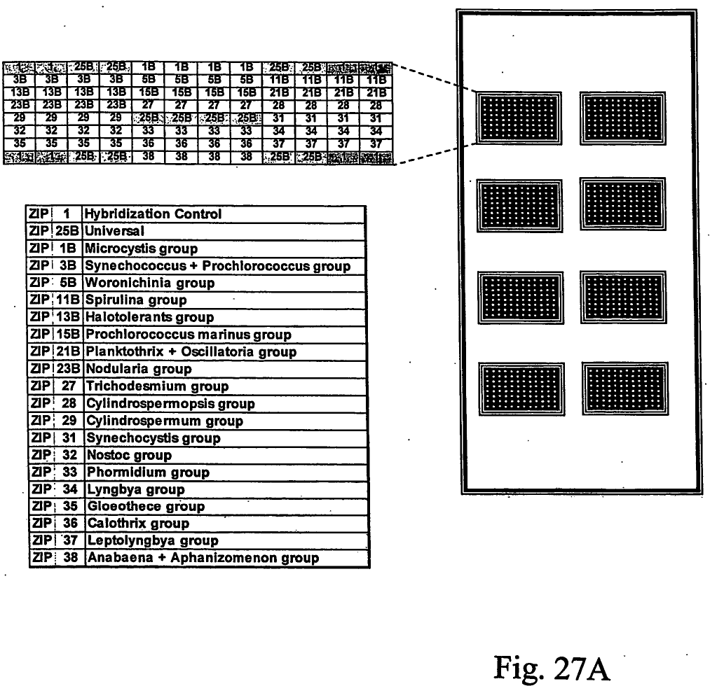

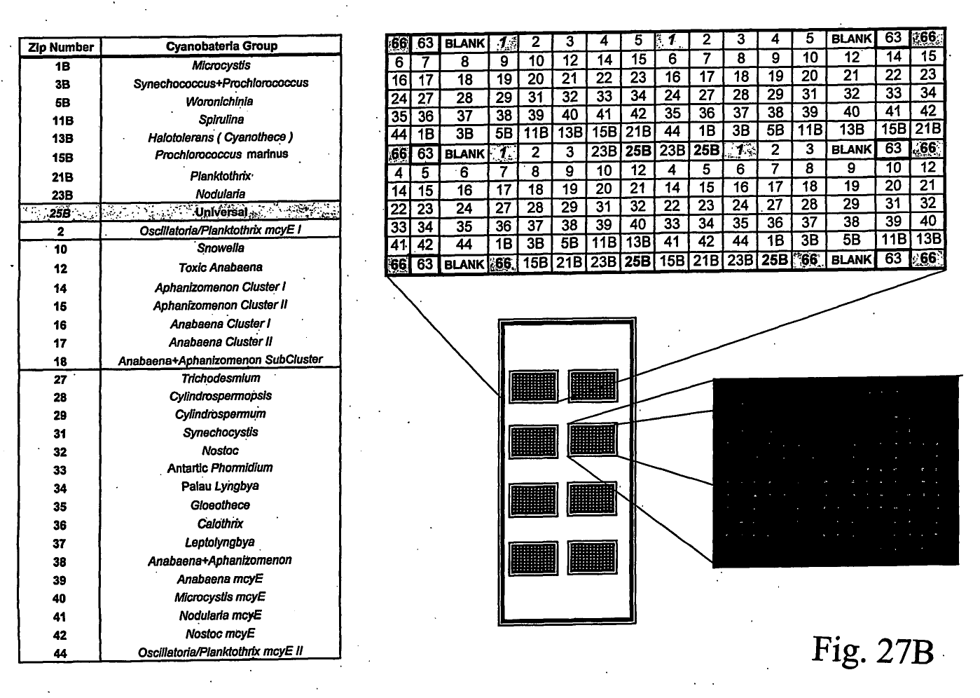

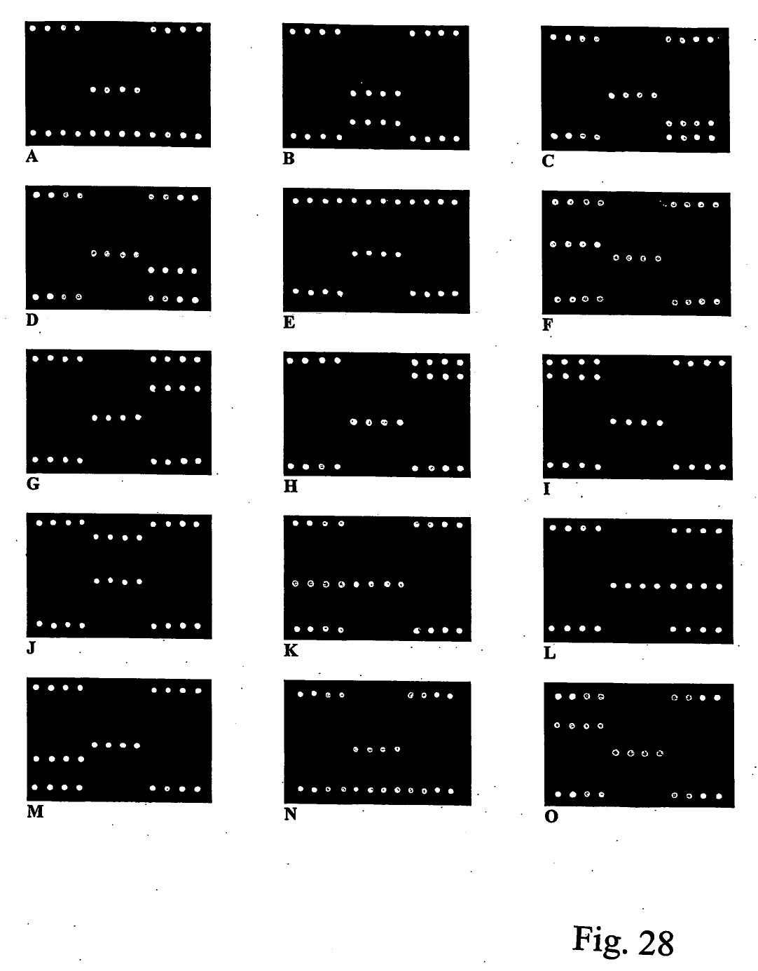

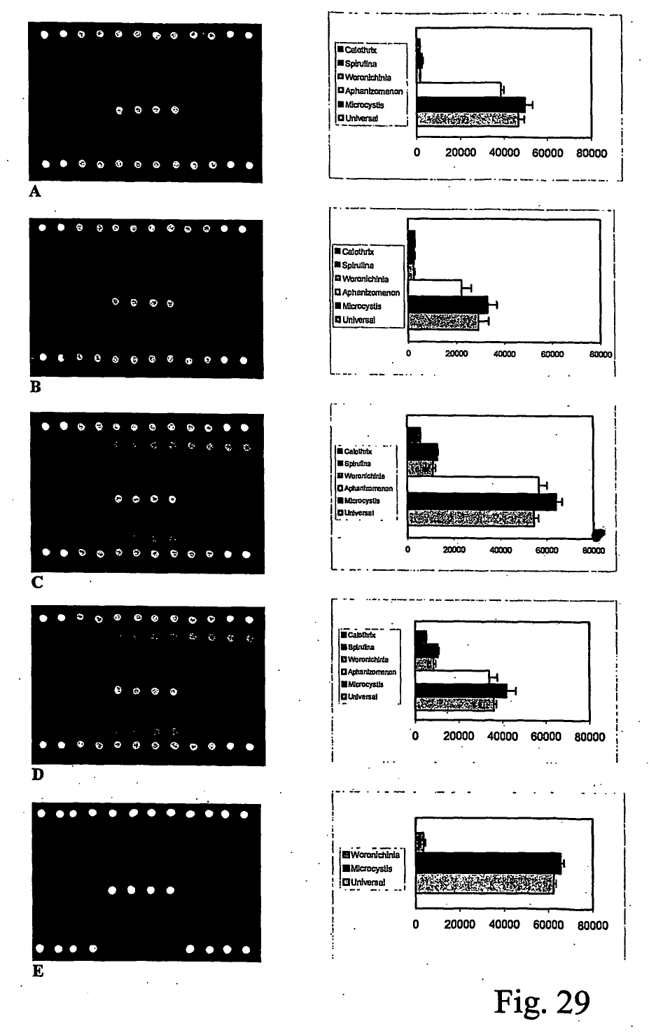

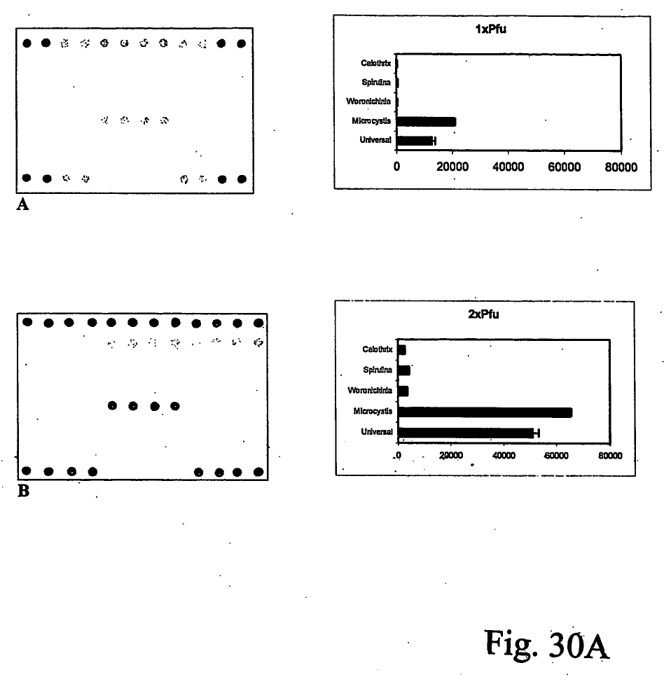

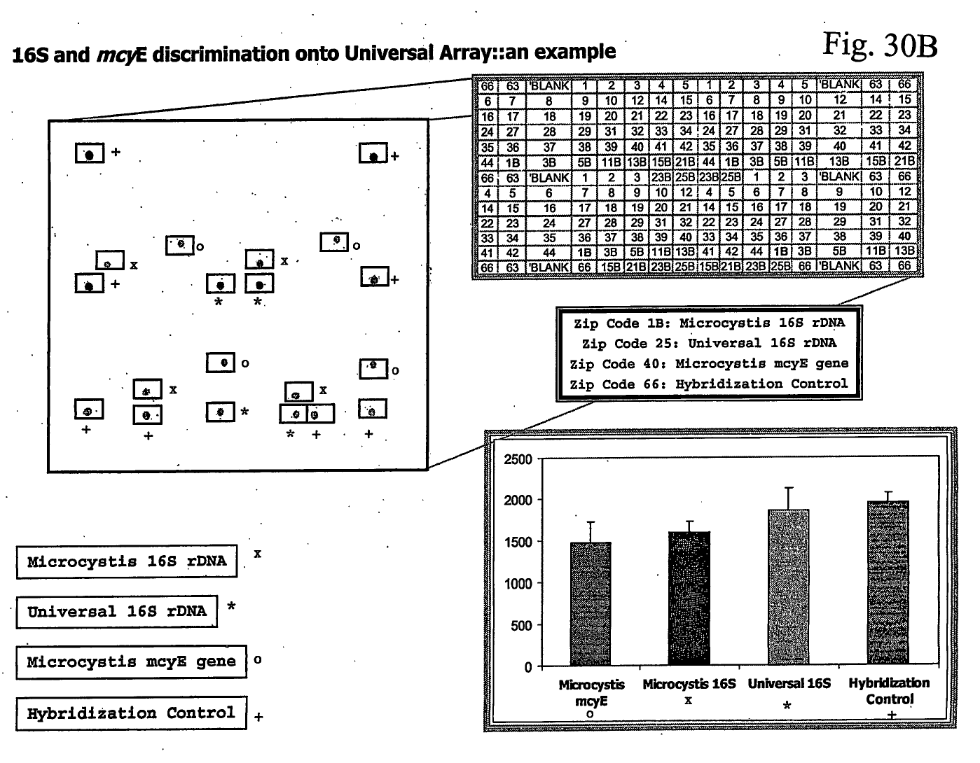

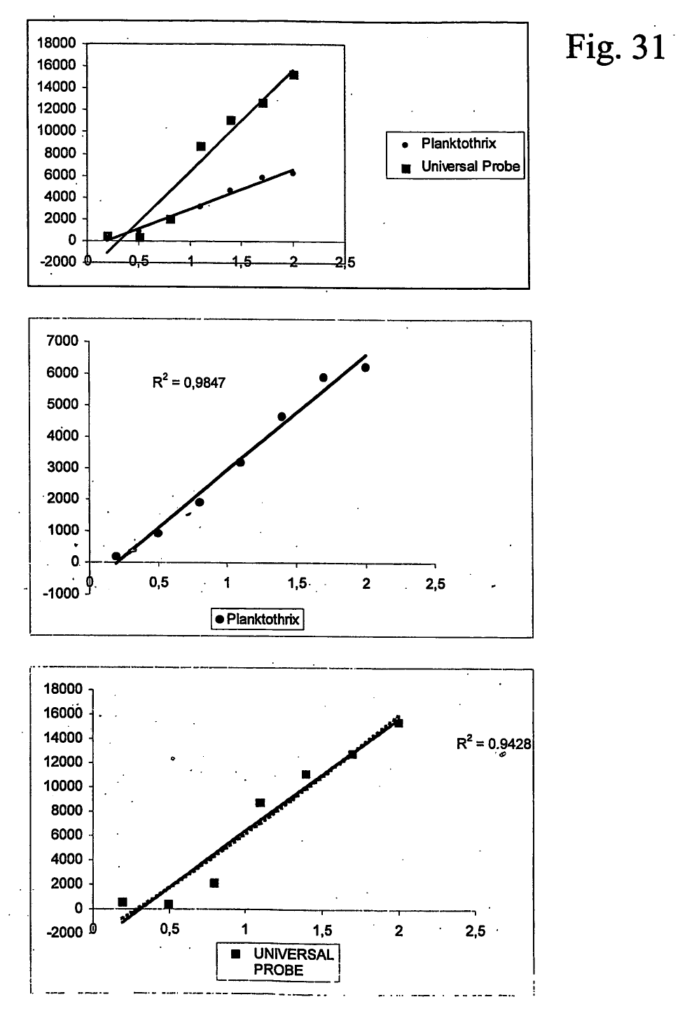

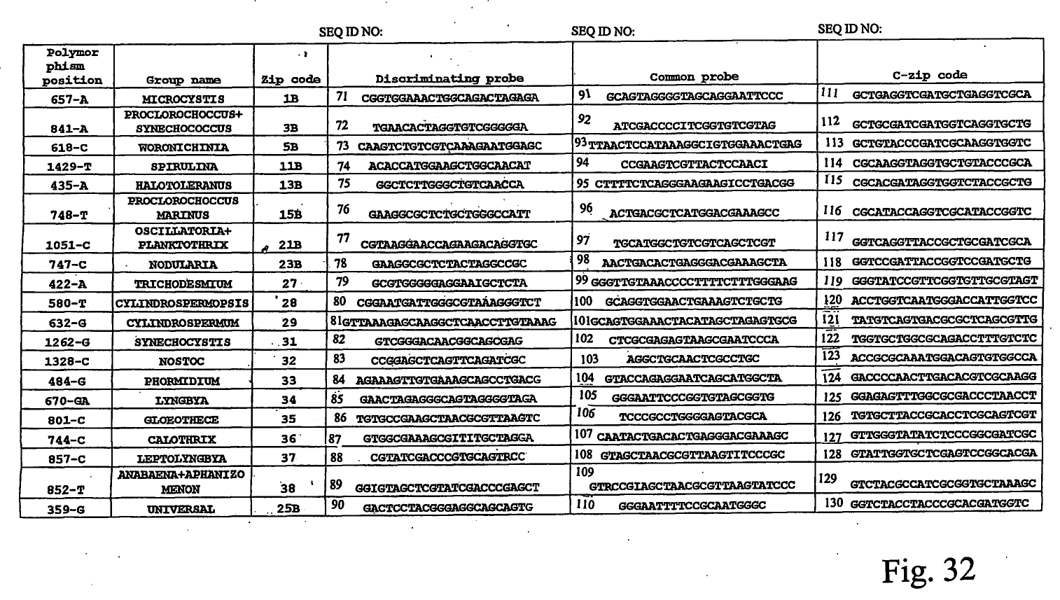

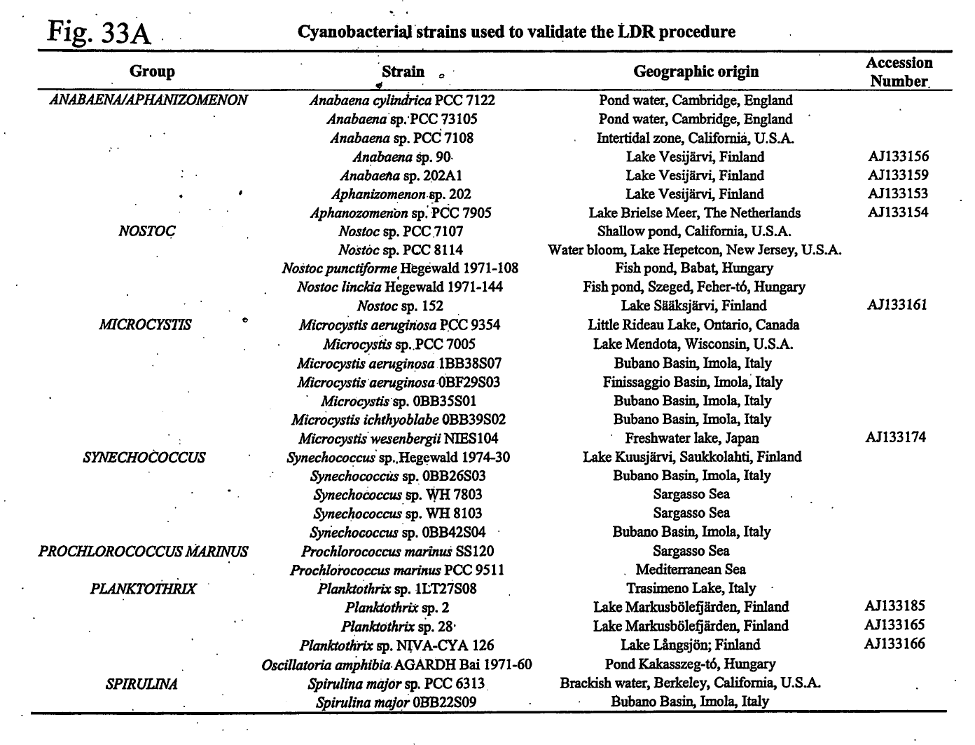

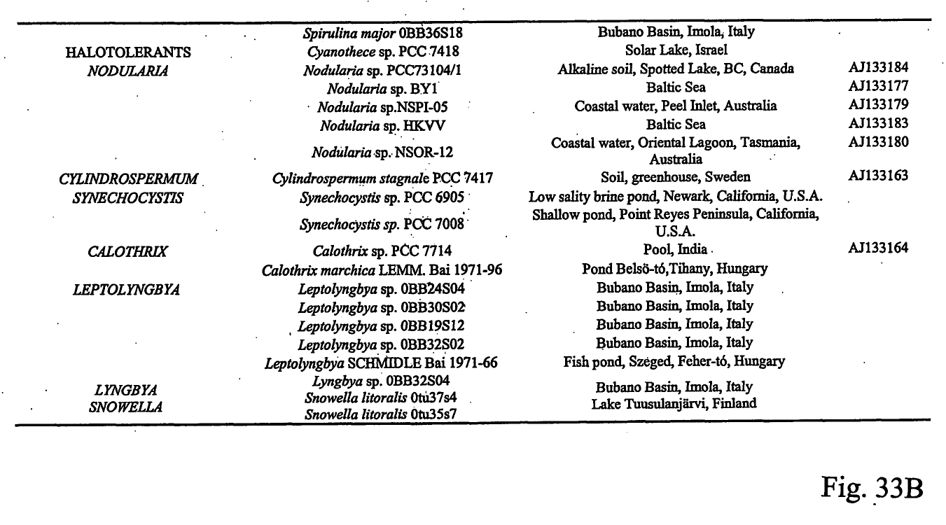

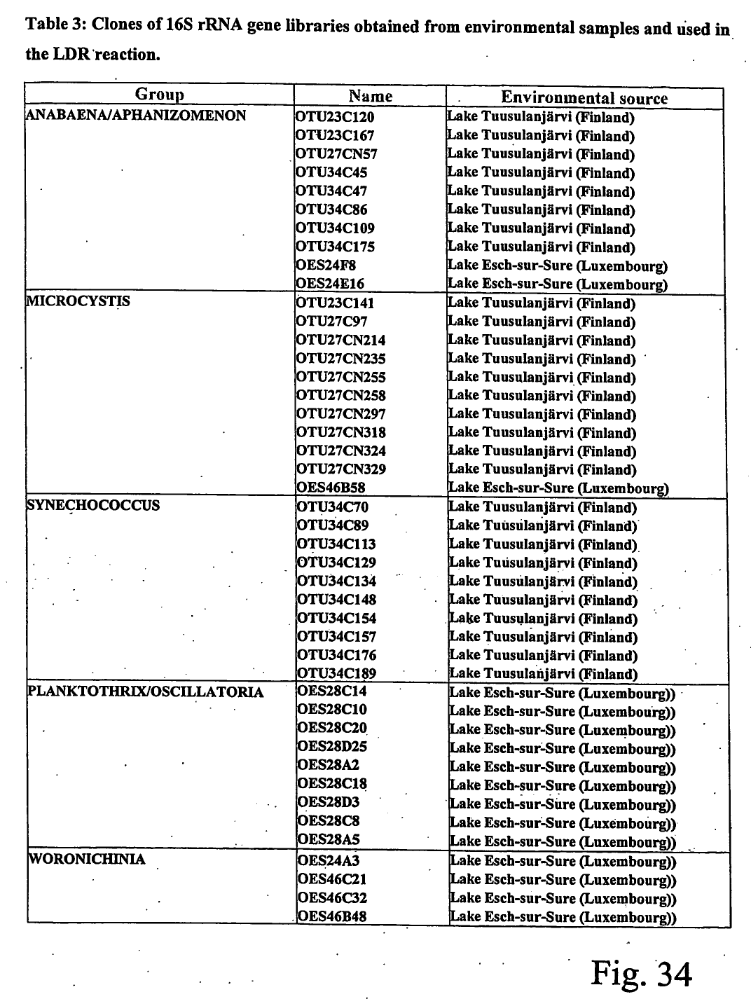

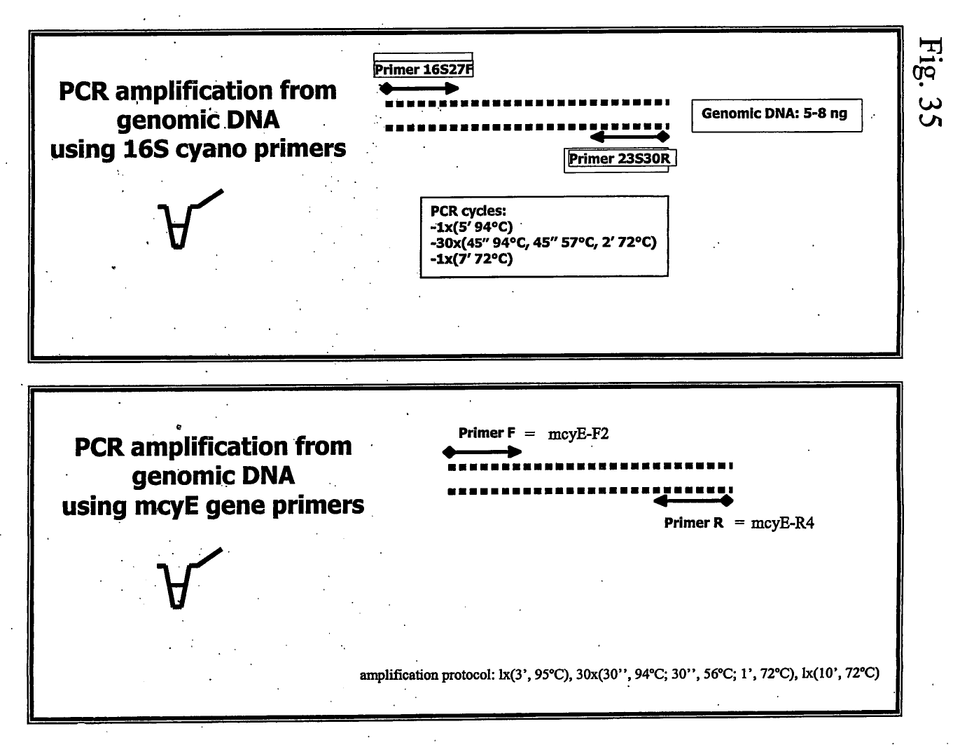

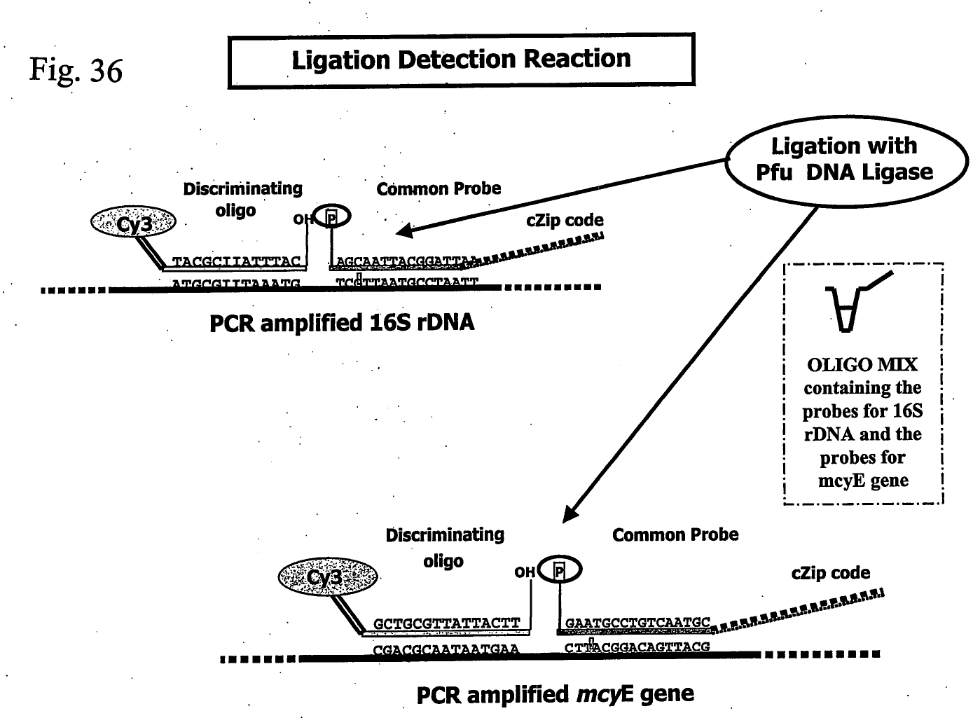

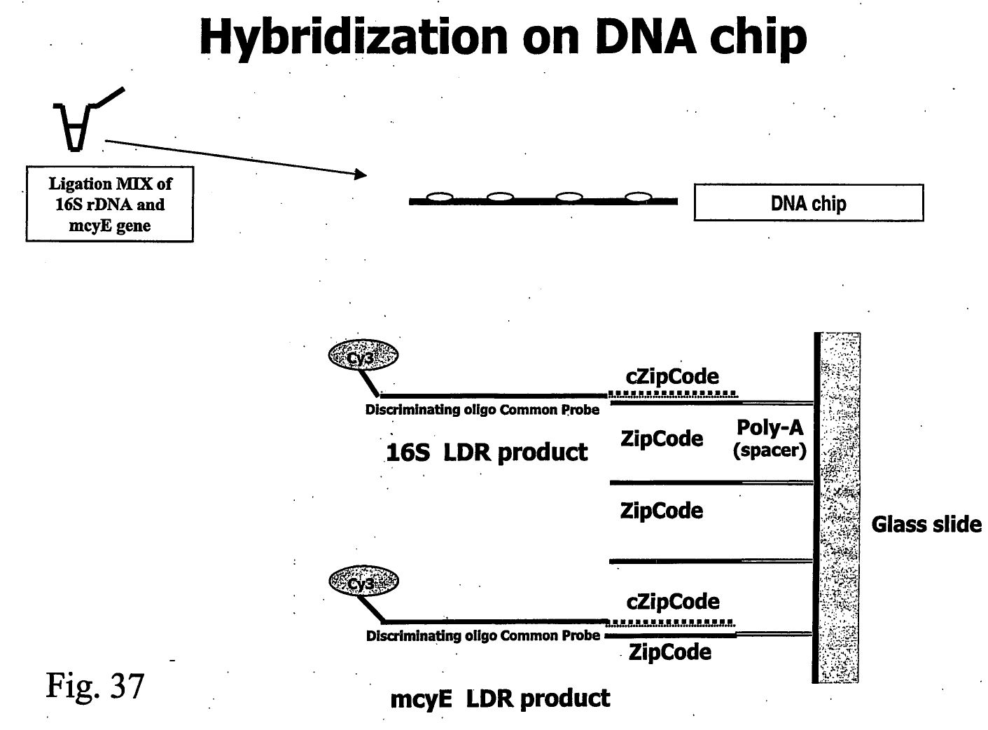

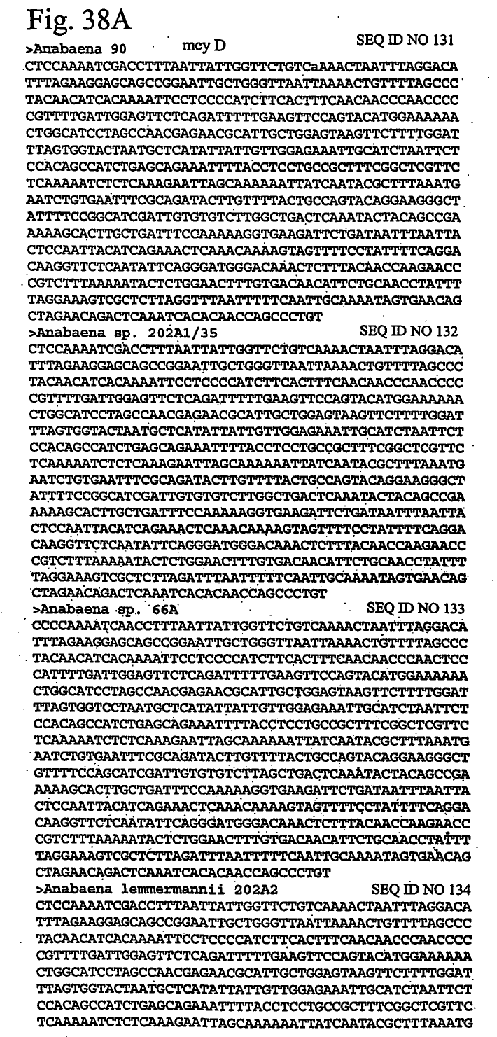

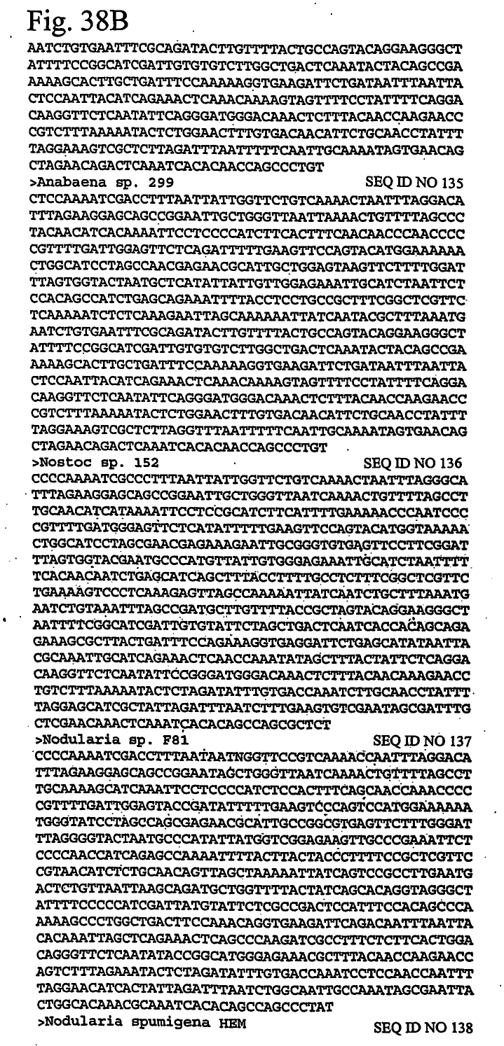

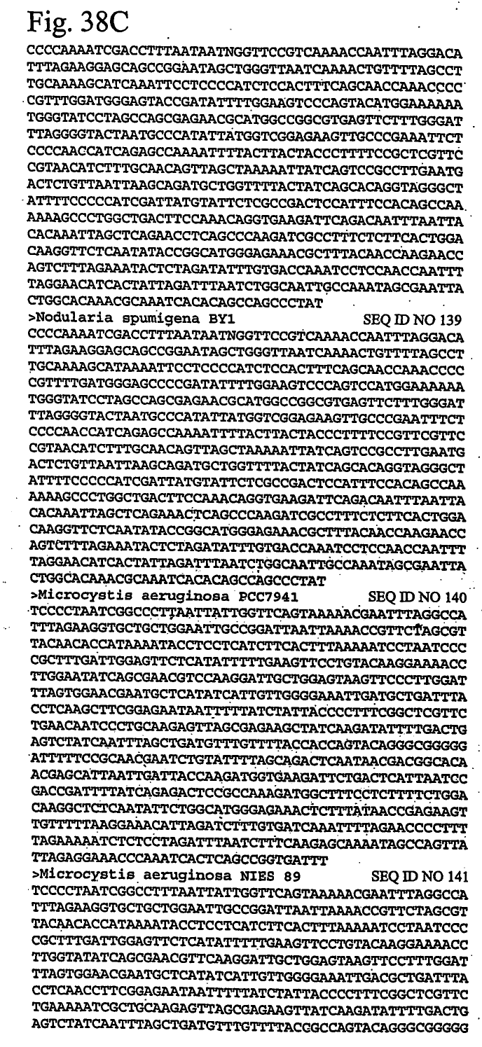

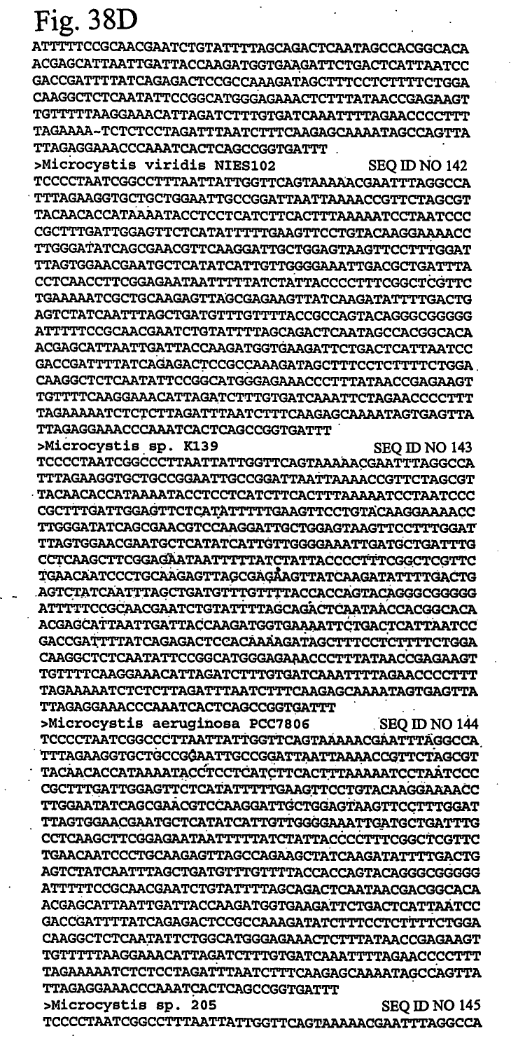

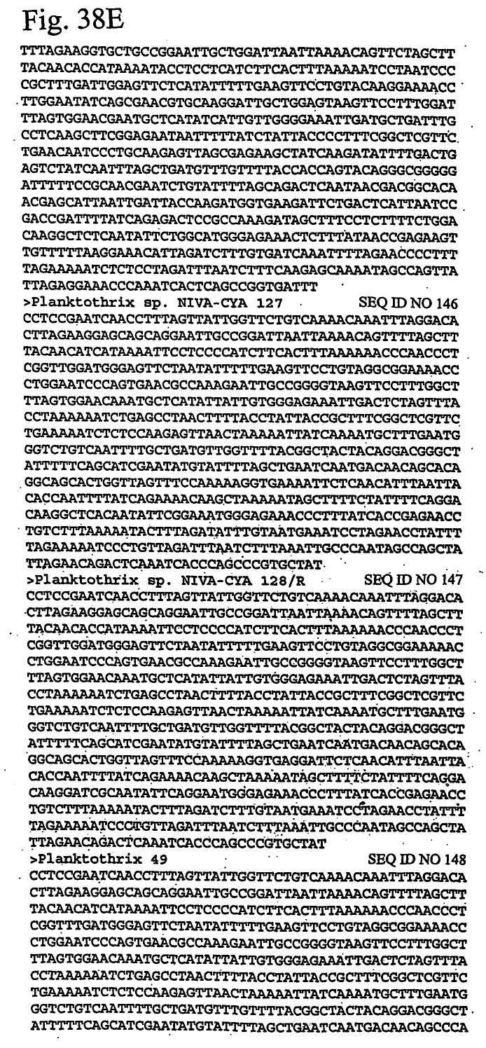

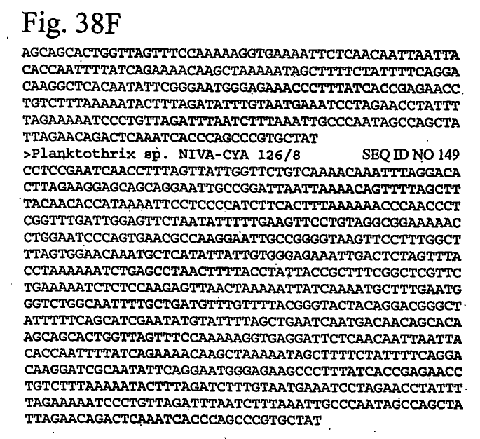

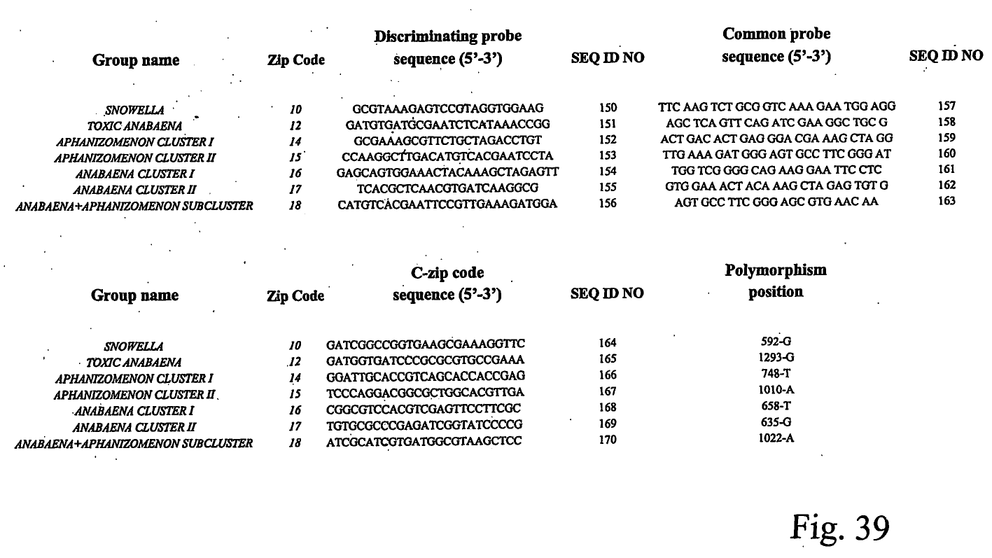

Motif scan at Prosite (Database of protein families and domains) and at Pfam (Protein families) database (http://hits.isb-sib.ch/cgi-bin/PFSCAN) and Conserved Domain (CD) search at NCBI (http://www.ncbi.nlm.nih.gov/Structure/cdd/wrpsb.cgi) were used to discover the putative functions of McyG, mcyD and McyE. In the N-terminal part of McyG a NRPS module was identified, which contains an adenylation domain and a thiolation (phosphopantetheine carrier) domain. Next to this, toward the C-termiinus there are four polyketide synthase (PKS) domains: β-ketoacyl synthase (KS), acyltransferase (AT), ketoreductase (KR) and acylcarrier protein (ACP), in this order. Between AT and KR domains there is a C-methyltransferase, MeT/CM) domain (FIG. 1). McyD contains two modules of the type I polyketide synthases. The first module consists of KS, AT, dehydratase (DH), MeT (CM) (FIG. 1), KR and ACP domains; and module two has KS, AT, DK KR and ACP domains, in the presented orders. mcyE is the other mixed PKS/NRPS, including PKS domains KS, AT, ACP and MeT (CM) (FIG. 1; FIG. 2A). These are followed by a unique aminotransferase domain (AMT) (FIG. 1; FIG. 2B) found in other microcystin synthetases (Tillet et al., 2000; Christiansen et al., 2003), and also in the synthetases of mycosubtilin (Duitman et al., 1999) and iturin (Tsuge et al., 2001) of Bacillus subtilis. At the N-terminal region, subsequently there is a NRPS module comprising of two condensation domains, an adenylation and a thiolation (peptidyl carrier) domain (FIG. 1).

Ketoreductase and Dehydratase Domains

The activity of the KR domains of McyG (one) and McyD (two) can be predicted from the microcystin synthetases structure, and they have the NAD cofactor binding motif, GXGXX(G/A)(X)₃(G/A)M(X)₆G, common to oxidoreductases (Scrutton et al., 1990). (FIG. 3B) The DH domains in the modules of McyD (AMCD-DH2 and AMCD-DH3) contain the active site motif H(X)₃D(X)₄P and H(X)₃G(X)₄P, respectively (FIG. 3A). The motif in AMCD-DH3 is identical to the consensus sequence (Aparicio et al., 1996). The motif H(X)₃D(X)₄P, where Gly is substituted by Asp, is also found in the active DH domain of module 10 in rifamycin synthase (Tang et al., 1998) (FIG. 3). This supports the conclusion based on the microcystin structure, that the DH domains in McyD are functional.

Specificity of the Acyl Transferase Domains

From the structure of the microcystins it is possible to conclude that the single AT domains of McyG and McyE, and the first AT domain of McyD, load methylmalonyl-CoA. But the presence of methyltransferase domains in McyG, McyD and mcyE wig. 1, FIG. 2A) suggests that the loading unit can be malonyl-CoA. Regions have been identified in AT domains, where the sequences are different depending on the specificity for either malonyl-CoA or methylmalonyl-CoA (FIG. 4) (Ikeda et al., 1999). By analysing the sequences of the acyltransferase domains (FIG. 4) and comparing them with the AT domains of soraphen and rapamycin synthases, which utilize malonyl subunits, we concluded that all the AT domains of microcystin synthetase load malonyl units. The methyltranferase domains of McyG, McyD and mcyE carry out three methylations in the positions indicated with arrows (FIG. 1). The CD search relates these domains to the UbiE/COQ5 C-methyltranferase family.

Ketosynthase and Acylcarrier Protein Domains

The active site cysteine and the two histidine residues which are present in polyketide synthases (Aparicio et al., 1996) were found in the KS domains of McyG, McyD and McyE (FIG. 5A). The only ACP domain of McyG and the first ACP domain of McyD have the active site sequence MGXDS, where a methionine residue replaces the commonly identified leucine residue (FIG. 5B). There are also variations in this position of the rifamycin synthase (rang et al., 1998). The ACP domain from the second module of McyD has the active site motif LGLNS (FIG. 5B), where Asn takes the place of the generally found Asp as in the module 11 of the rapamycin synthase (Aparicio et al., 1996).

The Order of the Genes in the Microcystin Synthetase Gene Cluster is Different in the Cyanobacterial Species

The arrangement of the genes is different in the gene clusters of microcystin biosynthesis from the strains of three species. In Anabaena strai 90, Microcystis aeruginosa (Tillett et al., 2000; Nishizawa et al., 2000) and in Planktothrix agardhii CYA126 (Christiansen et al., 2003) the NRPS genes, mcyA, mcyB and mcyC have the same order, but the organization of the other genes is different. In Anabaena strain 90 and in M. aeruginosa the mcy-genes are in two clusters, which are transcribed in opposite directions, whereas in P. agardhii they are in one cluster transcribed in the same direction (except mcyT, which was not found in Anabaena and Microcystis). The arrangement of the genes from mcyD to mcyH in Microcystis is almost identical in Planktothrix (mcyF is missing in Planktothrix), but it differs from the order in Anabaena. In Planktothrix, compared to Microcystis, the part containing mcyD, mcyE, mcyF, mcyG, mcyH, mcyI and mcyJ is reversed. In this rearrangement, mcyF and mcyI were lost from the cluster and mcyJ was relocated after mcyC.

The Biosynthesis of Microcystins

In Anabaena, the order of the domains coded by the genes in the two sets is co-linear with the hypothetical sequence of the enzymatic reactions for microcystin biosynthesis (FIG. 1). The progression of the biosynthetic reactions follows the order of the functions coded first by mcyG and continuing with the activities coded by mcyD, mcyJ, mcyE, mcyF, mcyI, mcyA, mcyB and mcyC.

Phenyl acetate is the assumed staring unit in the biosynthesis of Adda (Moore et al., 1991). It is activated by the adenylating domain identified in the N-terminus of McyG, and transferred onto the subsequent thiolation (phosphopantetheine binding) site. Polyketide synthesis reactions are followed (FIG. 1). All four extension units are malonyl-CoA molecules according to the substrate specificity of the AT domains (FIG. 4). In McyG there is a KS domain to catalyse the first condensation reaction between phenylacetate and malonyl-CoA.

The reductive reactions needed to fashion the polyketide chain are putatively catalysed by KR and DH domains of McyD and McyE. The KR domain of McyG is in the right position to reduce the carbonyl group of the putative starter molecule. The methyltransferase domains of McyG, McyD and mcyE are the obvious candidates to introduce three methyl groups into the carbon frame of Adda. It was recently verified with a knockout mutant (Christiansen et al., 2003) that the incorporation of the fourth methyl, which is seen in the methoxy group of Adda, is catalysed by McyJ. The amino transferase domain of mcyE most likely adds the amino group, which participates in the peptide bond with the glutamate residue.

There are two condensation domains of peptide synthetases in McyE. The first one logically catalyses the peptide bond between Adda and glutamate, which is activated by the adenylation domain of McyE. The signature sequence, which was also determined as DPRHSGVVG for McyE of both M. aeruginosa and P. agardhii, has no precedents in the databases (Table 2). The synthetases of other peptides, which contain glutaryl residues are known for bacitracin, fengycin and surfactin (accession numbers: AF007865, AF023464, AF087452 and D13262). In these compounds the standard α-carboxyl of glutamate is part of the peptide bond, while in microcystins it is the γ-carboxyl. This is analogous to the activation of aspartate/methylaspartate by the second adenylation domain of McyB. The β-carboxyl of aspartate/methylaspartate instead of the α-carboxyl is engaged in the peptide bond formation. This must have impact on the compositions of the glutamate and aspartate/methylaspartate binding pockets in the adenylation domains.

McyA has two adenylation domains for the activation of serine and alanine, respectively. The signature sequences of these domains have models and are almost identical in Anabaena 90, M. aeruginosa and P. agardhhi (Table 2). The dehydration of serine supposedly takes place after the activation by adenylation and is catalysed by McyI, which is similar to phosphoglycerate dehydrogenases.

There is only one, internal, condensation domain in McyA, which most likely links dehydroserine and D-alanine. The bond between glutamate and dehydroserine is putatively catalysed by the C-terminal condensation domain of McyE. There is a methyltransferase domain in the first module of McyA for N-methylation of dehydroserine. The epimerase domain at the C-terminus of McyA converts L-alanine to the D-form.

Two modules of McyB and one module of McyC logically activate, and add three residues to the nascent peptide chain: L-leucine or L-arginine, methylaspartate or aspartate and L-arginine, respectively (FIG. 1). The amino acids activated by the adenylation domains of McyC and by the first module of McyB (McyB-1) vary most frequently in microcystins. M. aeruginosa PCC7806 and M. aeruginosa K-139 produce mainly Mcyst-LR, and the substrate specificity conferring sequences in McyB-1 of these strains are identical with the signature sequence for leucine (Table 2). M. aeruginosa UV027 and P. agardhii CYA126 produce mostly Mcyst-RR, which is also produced by Anabaena 90 together with Mcyst-LR. Their signature sequences in McyB-1 are different and have no precedents in the databases (Table 2). In M. aeruginosa UV027 the specificity codes of McyB-1 and McyC are almost identical (DVWTIGAVE/DWTIGAVD) and match with the codes of McyC from M. aeruginosa K-139 and M. aeruginosa PCC7806, respectively (Table 2). Accordingly McyB-1 of M. aeruginosa UV027 and McyC activate arginine.

There is no epimerase domain in McyB of Anabaena 90 or in the other sequenced versions of McyB, though in microcystins, the aspartyl or methylaspartyl moiety is in the D-form. The epimerization in this position and in the glutamyl residue is putatively catalysed by McyF, which in a BLAST search was similar to aspartate racemases, and was shown by Nishizawa et al., (2001) to complement a D-glutamate deficient mutant of Eschericia coli. The C-terminal thiosterase domain of McC, as generally in nonribosomal peptide synthesis, (Kohli et al., 2001) catalyzes the final step in microcystin biosynthesis, the cyclization of the linear peptide (FIG. 1).

McyH is probably not needed for the synthesis of microcystins but it may participate in the transport of microcystins.

### Example 2

Taxon Sampling, Amplification and Sequencing

Genomic DNA from 36 strains of Anabaena, Microcystis, Planktothrix, Nodularia, and Nostoc was extracted. We chose three regions of the microcystin synthetase gene cluster to study the evolution of this biosynthetic system in cyanobacteria. A fragment of 291-297 bp from the mcyA gene was amplified with mcyA-Cd 1R (5′-aaaagtgttttattagcggctcat-3′) and mcyA-Cd 1F (5′-aaaattaaaagccgtatcaaa-3′) primers and sequenced as described earlier (Hisbergues et al. 2003). An 818 bp region of the mcyD gene was amplified with mcyDF (5′-gatccgattgaattagaaag-3′) and mcyDR (5′-gtattccccaagattgcc-3′) primers. An 809-812 bp region of the mcyE gene was amplified with the mcyE-F2 (5′-gaaatttgtgtagaaggtgc-3′) and mcyE-R4 (5′-aattctaaagcccaaagacg-3′) primers. The mcyE PCR products of Nodularia sp. strains were cloned with the TOPO TA cloning kit (Invitrogen) according to the manufacturer's instructions. The rpoC1 gene fragment of 750 bp was amplified with degenerate primers RF (5′-tgggghgaaagnacaytncctaa-3′) and RR (5′-gcaaancgtccnccatcyaaytgba-3′). PCR reactions for mcyE, mcyD and rpoC1 were performed in a 20 μl final volume containing 1 μl of DNA, 1×DynaZyme II PCR buffer, 250 μM of each deoxynucleotide, 0.5 μM of both PCR primers, and 0.5 U of DynaZyme II DNA polymerase (Finnzymes, Espoo, Finland). The following protocol was used: 95° C., 3 min; 30×(94° C., 30 sec; 56° C., 30 sec; 72° C., 1 min); 72° C., 10 min. A region containing the 16S rRNA gene and the internal transcribed spacer 1 (ITS1) was amplified using primers and conditions described earlier (Lepére et al., 2000) from strains, for which the 16S rRNA sequence data was not available. The mcyD and mcyE gene products were sequenced directly with primers used for amplification except for the cloned mcyE sequences of Nodulria sp. strains, which were sequenced with primers anchored in the pCR2.1-TOPO vector, M13F (−20) and M13R. The rpoC1 gene products were sequenced with the amplification primers and with two additional internal sequencing primers RintF (5′-gatatgcccctgcgggatgt-3′) and RintR (5′-acatcccgcaggggcatatc-3′). The 16S rRNA gene region of the amplified PCR products was sequenced directly using sets of internal primers (Edwards et al., 1989).

Sequencing of the mcyD, mcyE and 16S rRNA genes was performed by Genome Express (France). The rpoC1 products were sequenced with ABI PRISM 310 Genetic Analyzer. The mcyA sequences were assembled as described by Hisbergues at al. The chromatograms of mcyD, mcyE, rpoC1 and 16S rRNA gene sequences were checked and edited with Chromas 2.2 program (Technelysium Pty Ltd.). Contig assembly and alignment of the sequences were performed with BioEdit Sequence Alignment Editor (Hall et al., 1999).

Phylogenetic Analyses

Primer sequences and ambiguous regions of the alignments were excluded. The aligned data sets were the following lengths: mcyA (99 amino acids), mcyD (286 amino acids), mcyE (270 amino acids), rpoC1 (750 bp) and 16S rRNA (1455 bp). These sequences were combined with the sequence available from Microcystis aeruginosa PCC 7806 (Tillett et al., 2000). and Planktothrix agardhii NIVA-CYA 126/8 (Christiansen et al., 2003).

Outgroups for each of the three microcystin synthetase genes were identified with BLAST searches (Supplementary Information). We aligned mcyA, mcyE, and mcyD and the top three hits in BLAST searches with BioEdit (Hall et al. 1999).

Only conserved and reliably aligned sequence regions from the outgroup sequences were used in order to minimise potential phylogenetic reconstruction artefacts derived from the use of distant outgroups Swofford et al. 1996). In order to assess the stability of the ingroup tree topology, which could be influenced by the addition of outgroup lineages due to long branch attraction, the phylogenetic trees were analysed with and without the chosen outgroups. Phylogenetic analyses were performed with PAUP (Swofford, 2001) and PHYLIP (Felsenstein, 1993). Maximum likelihood and maximum parsimony analyses were used to reconstruct trees from each mcy gene fragment, and to compare the tree topologies of the separate and concatenated mcy gene sets and the 16S rRNA and rpoC1 genes. 16S rRNA sequences of 53 cyanobacterial strains and three outgroup species were used to construct a maximum-likelihood tree, to which the distribution of microcystin and nodularin producing cyanobacteria among other cyanobacteria was mapped (FIG. 8).

TABLE 3
Accession numbers for sequences used in phylogenetic reconstruction. A solid
line denotes unsuccessful attempts to amplify this region from the three strains of
the genus Nodularia used in this study. A dash indicates cases where
no attempt was made to obtain sequence data.

| Taxon | mcyA | mcyE | mcyD | 16S rRNA | rpoC1 |
|---|---|---|---|---|---|
| Microcystis sp. HUB 5-2-4 | AJ515451 | — | — | — | — |
| Microcystis aeruginosa NIES 89 | AJ515459 | AY382530 | AY424988 | U03403 | — |
| Microcystis sp. 199 | AJ515452 | — | — | AJ133172 | — |
| Microcystis sp. GL260735 | AJ515454 | AY382531 | — | AY439282 | — |
| Microcystis sp. GL280646 | AJ515455 | AY382532 | — | — | — |
| Microcystis sp. IZANCYA5 | AJ515456 | AY382533 | — | — | — |
| Microcystis sp. IZANCYA25 | — | AY382534 | — | — | — |
| Microcystis sp. TuM7C | AJ515458 | — | — | — | — |
| Microcystis viridis NIES 102 | AJ515457 | AY382535 | AY424991 | U40332 | AY425001 |
| Microcystis aeruginosa PCC 7941 | AJ515460 | AY382536 | AY424989 | U40340 | — |
| Microcystis aeruginosa PCC 7806 | AF183408 | AF183408 | AF183408 | AF139299 | AY425000 |
| Microcystis sp. 98 | — | AY382537 | — | — | — |
| Microcystis sp. 205 | AJ515453 | AY382538 | AY424990 | AY439281 | — |
| Nostoc sp. 152 | AJ515475 | AY382539 | AY424984 | AJ133161 | AY424997 |
| Nodularia spumigena HEM | | AY382540 | AY424985 | AF268005 | AY424999 |
| Nodularia spumigena BY1 | | AY382541 | AY424987 | AF268004 | — |
| Nodularia sp. F81 | | AY382542 | AY424986 | AY439283 | AY424998 |
| Anabaena sp. 66A | AJ515462 | AY382543 | AY424983 | AJ133157 | — |
| Anabaena sp. 66B | AJ515463 | — | — | — | — |
| Anabaena flos-aquae NIVA- | AJ515466 | AY382544 | — | AJ133158 | — |
| CYA83/1 | | | | | |
| Anabaena sp. 202A1/35 | AJ515464 | AY382545 | AY424980 | AJ133159 | — |
| Anabaena lemmermannii 202A2 | AJ515465 | AY382546 | AY424981 | AJ293104 | AY424995 |
| Anabaena sp. 90 | AJ515461 | AJ536156 | AJ536156 | AJ133156 | AY424996 |
| Anabaena sp. PH256 | — | AY382547 | — | — | — |
| Anabaena sp. 315 | — | AY382548 | — | — | — |
| Anabaena sp. 318 | — | AY382549 | — | — | — |
| Anabaena sp. 299 | — | AY382550 | AY424982 | AJ293106 | — |
| Planktothrix sp. HUB 076 | AJ515472 | — | — | — | — |
| Planktothrix sp. PCC7821 | AJ515473 | — | — | — | — |
| Planktothrix sp. NIVA-CYA34 | AJ515474 | — | — | — | — |
| Planktothrix sp. 49 | AJ515470 | AY382551 | AY424992 | AJ133167 | AY425003 |
| Planktothrix sp. 97 | AJ515471 | AY382552 | — | — | — |
| Planktothrix sp. NIVA-CYA126 | AJ441056 | AJ441056 | AJ441056 | AJ133166 | — |
| Planktothrix sp. NIVA-CYA127 | AJ515468 | AY382553 | AY424993 | AJ133168 | AY425002 |
| Planktothrix sp. NIVA- | AJ515469 | AY382554 | AY424994 | AJ133169 | — |
| CYA128/R | | | | | |
| Oscillatoria sp. 213 | — | AY382555 | — | — | — |
| Oscillatoria sp. 226 | — | AY382556 | — | — | — |

TABLE 4
Accession numbers of sequences used to root the microcystin gene data set in FIG. 7.
The outgroup sequences identified by BLAST searches were fused together to form three
outgroup sequences in the mcyA, mcyD, and mcyE concatenated gene data set.

| Gene | Outgroup | Accession | Organism | Gene | Function |
|---|---|---|---|---|---|
| McyA | Outgroup 1 | AF210249 | Streptomyces verticillus | blmX | Bleomycin biosynthetic gene |
| | Outgroup 2 | AE004755 | Pseudomonas aeruginosa | PA3327 | Probable non-ribosomal peptide |
| | | | | | synthetase |
| | Outgroup 3 | X97860 | Amycolatopsis mediterranei | aps | Peptide-synthetase |
| McyD | Outgroup 1 | AF395828 | Aphanizomenon ovalisporum | aoaC | Polyketide synthase |
| | Outgroup 2 | AJ421825 | Stigmatella aurantiaca | stiH | Stigmatellin biosynthetic gene |
| | Outgroup 3 | AP003590 | Nostoc sp. PCC 7120 | alr2680 | Polyketide synthetase |
| McyE | Outgroup 1 | D29676 | Bacillus brevis | Grs2 | Gramicidin S synthetase 2 |
| | Outgroup 2 | X70356 | Bacillus subtilis | sifA1 | Surfactin synthetase |
| | Outgroup 3 | AF004835 | Brevibacillus brevis | tycC | tyrocidine synthetase 3 |

TABLE 5
Accession numbers for 16S rRNA sequences used to construct
the maximum-likelihood tree presented in FIG. 8.

| Species | Strain | 16S rRNA |
|---|---|---|
| Cyanobacteria | | |
| Subsection I Chroococcales | | |
| Cyanobium gracile | PCC 6307 | AF001477 |
| Cyanothece sp. | PCC 7424 | AF132932 |
| Gloeobacter violaceus | PCC 7421 | AF132790 |
| Gloeothece membranacea | PCC 6501 | X78680 |
| Microcystis aeruginosa | PCC 7806 | U03402 |
| Microcystis aeruginosa | PCC 7941 | U40340 |
| Microcystis wesenbergii | NIES 104 | AJ133174 |
| Synechococcus elongatus | PCC 6301 | X03538 |
| Synechococcus leopoliensis | PCC 7942 | AF132930 |
| Synechococcus sp. | PCC 7002 | AJ000716 |
| Synechococcus sp. | PCC 6716 | AF216942 |
| Synechococcus sp. | WH 8103 | AF311293 |
| Synechocystis sp. | PCC 6803 | D64000 |
| Thermosynechococcus elongatus | BP-1 | AP005376 |
| Prochlorococcus marinus | MED 4 | AF001466 |
| Prochlorococcus marinus | MIT 9313 | AF053399 |
| Subsection II Pleurocapsales | | |
| Chroococcidiopsis sp. | SAG 2023 | AJ344552 |
| Chroococcidiopsis thermalis | PCC 7203 | AB039005 |
| Myxosarcina sp. | PCC 7312 | AJ344561 |
| Myxosarcina sp. | PCC 7325 | AJ344562 |
| Pleurocapsa minor | SAG 4.99 | AJ344564 |
| Pleurocapsa sp. | PCC 7516 | X78681 |
| Xenococcus sp. | PCC 7305 | AF132783 |
| Subsection III Oscillatoriales | | |
| Arthrospira sp. | PCC 8005 | X70769 |
| Leptolyngbya sp. | PCC 7375 | AF132786 |
| Leptolyngbya sp. | PCC 7104 | AB039012 |
| Limnothrix redekei | NIVA-CYA 227/1 | AB045929 |
| Lygnbya aestuarii | PCC 7419 | AJ000714 |
| Oscillatoria rosea | IAM-220 | AB003164 |
| Oscillatoria sancta | PCC 7515 | AF132933 |
| Planktothrix agardhii | NIVA-CYA 126 | AJ133166 |
| Planktothrix sp. | 2 | AJ133185 |
| Planktothrix sp. | 49 | AJ133167 |
| Pseudanabaena sp. | PCC 6903 | AF132778 |
| Spirulina major | PCC 6313 | X75045 |
| Spirulina subsalsa | IAM-223 | AB003166 |
| Trichodesmium erythraeum | IMS101 | Unpublished* |
| Prochlorothrix hollandica | — | AF132792 |
| Subsection IV Nostocales | | |
| Anabaena sp. | 66A | AJ133157 |
| Anabaena sp. | 90 | AJ133156 |
| Anabaenopsis circularis | NIES 21 | AF247595 |
| Anabaenopsis sp. | PCC 9215 | AY038033 |
| Aphanizomenon flos-aquae | NIES 81 | AJ293131 |
| Cyanospira rippkae | PCC 9501 | AY038036 |
| Cylindrospermum stagnale | PCC 7417 | AF132789 |
| Nodularia spumigena | BY1 | AF268004 |
| Nodularia sp. | F81 | AY439283 |
| Nodularla spumigena | PCC 73104 | AF268023 |
| Nostoc sp. | PCC 7120 | X59559 |
| Nostoc punctiforme | PCC 73102 | AF027655 |
| Nostoc sp. | 152 | AJ133161 |
| Nostoc sp. | PCC 9709 | AF027654 |
| Scytonema hofmannii | PCC 7110 | AF132781 |
| Subsection V Stigonematales | | |
| Chlorogloeopsis sp. | PCC 7518 | X68780 |
| Fischerella muscicola | PCC 7414 | AP132788 |
| Outgroups | | |
| Bacillus subtilis | BS62 | AB016721 |
| Chlorobium tepidum | — | M58468 |
| Escherichia coli | K12 | AE000129 |

*Unpublished 16S rRNA obtained from Trichodesmium erythraeum IMS101 on the Joint Genome Institute webpage (www.jgi.doe.gov).

### Example 3

Primer design and specificity testing. General microcystin synthetase E forward primer (mcyE-F2) and genus specific reverse primers for Anabaena (AnamcyE-12R) as well as for Microcystis (MicmcyE-R8) (able 6) were designed with mcy gene sequences of Anabaena 90 (see Example 1), by using BLAST (1) and BioEdit (Hall 1999).

Specificity of these primers was tested with 14 Anabaena, 13 Microcystis, 8 Planktothrix strains and with one Nostoc strain (Table 7). Microcystis and Planktothrix strains were grown in Z8 medium (Kotai 1972), whereas Anabaena and Nostoc strains were grown in a modified Z8 medium without nitrogen. The strains were grown under continuous light (20 μmol m⁻²s⁻¹) at 20±2° C.

PCR reaction was carried out with 1 μl of extracted DNA, 1×DynaZyme II PCR buffer [10 mM Tris-HCl, pH 8.8 at 25° C., 1.5 mM MgC₂, 50 mM KCl, 0.1% Triton X-100, (Finnzymes)], 250 μM dNTPs (Finnzymes), 0.5 μM of primers (Sigma-Genosys Ltd.) and 0.5 U of DyNAzyme II DNA polymerase (Finnzymes) in a volume of 20 μL. The PCR amplification was performed with initial denaturation at 95° C. for 3 min followed by either 30 (Anabaena) or 25 (Microcystis) cycles at 94° C. for 30 s, at 58° C. for Anabaena and at 60° C. for Microcystis for 30 s and at 72° C. for 60 s, followed by 10 min final extension at 72° C. Presence or absence of the mcyE product was determined using 20 μl of amplification product and 1.5% agarose gel electrophoresis.

Lake water samples. Water samples were collected at Lake Tuusulanjärvi from 0 to 2 m depth every second or third week during summer period 1999. For DNA extraction one liter of lake water was concentrated to less than 2 ml by centrifugation and stored at −70° C. Lake Hiidenvesi consists of several natural basins representing a transition from hypertrophy to mesotrophy. Water samples were collected from 3 to 5 different depths from basins of Kirkkojärvi (3.5 m deep at the sampling site), Mustionselkä (4 m), Nummelanselkä (6 m), and Kiihkelyksenselkä (30 m) on 15 Aug. 2001. For DNA extraction 100 ml of lake water was filtered through 3 μm pore size Poretics® polycarbonate disc filter (47 mm), (Osmonics Inc.) and cells were stored with lysis buffer at −20° C. (14). For microcystin concentration analysis, 5 ml of lake water was stored in a glass vial at −20° C. Cyanobacterial cell densities were determined using the inverted microscope technique (Utermöhl, 1958) from the samples which were preserved with acid Lugol's solution (Willen, 1962) and stored in darkness at 4° C.

Isolation and purification of DNAs. Genomic DNAs of the Anabaena, Microcystis, Planktothrix and Nostoc strains and the lake water samples were extracted with a hot phenol-chloroform-isoamylalcohol-method (Giovannoni et al., 1990). Extracted DNAs were purified either once (strains) or twice (lake water samples) with Prep-A-Gene® DNA Purification Systems (Bio-Rad) according to the manufacturer's instructions and eluted in 60 μl.

QRT-PCR. External standards for mcyE copy number quantification were prepared using genomic DNAs of strains Anabaena 90, 315, and 202A1 as well as those of Microcystis GL 260735, PCC 7806, and PCC 7941. Genomic DNA concentration of these DNAs was measured with a spectrophotometer at 260 nm (Beckman DU-7400). Purity was determined by calculating the ratio of the absorbances measured at 260 nm and 280 nm. Approximate genome sizes, Anabaena 5.15 Mb and Microcystis 4.70 Mb, were used in mcyE copy number calculation. These genome sizes were estimated based on the genome sizes of Anabaena PCC 6309, Anabaena PCC 7122 and Microcystis PCC 7941 (Castenholz, 2001). The mcyE copy numbers of the standard strains DNAs were calculated using following equation with the assumption that each genome had only one mcyE gene and the molecular weight of one bp was 660 g mol⁻¹:

$$Copies {μl}^{- 1} = \frac{6 \times {10}^{23} [ copies {mol}^{- 1} ] \times DNA concentration [ g {μl}^{- 1} ]}{Molecular weight of one genome [ g {mol}^{- 1} ]} . \qquad Equation 1$$

Ten-fold dilution series of genomic DNAs of the standard strains were prepared and these dilutions were amplified with Anabaena and Microcystis mcyE QRT-PCR. Linear regression equations of the obtained cycle threshold values (Ct values, i.e. the first turning points of the fluorescence curves as a function of cycle numbers) were calculated as a function of known mcyE copy numbers.

The QRT-PCR reaction was carried out with 1 μl of DNA of standard strains or lake water samples, 3 mM MgCl₂, 0.5 μM of both primers (Sigma-Genosys Ltd.) and 1 μl of hot start reaction mix to a final volume of 10 μl (LightCycler—fastStart DNA master SYBR green I—kit, Roche Diagnostics). Amplification was performed with initial preheating of 10 min at 95° C. followed by 45 cycles at 95° C. for 2 s, at 58° C. for 5 s and at 72° C. for 10 s. Generation of the products was monitored after each extension step at 77° C. in Anabaena and 78° C. in Microcystis mcyE QRT-PCR by measuring fluorescence of double-stranded DNA binding SYBR green 1 dye using LightCycler QRT-PCR (Roche Diagnostics). All lake water samples were amplified three times. The Ct values were determined by the second derivative maximum method of LightCycler software (version 3.5). Copy numbers of mcyE gene of the lake water samples were determined by converting obtained Ct values into the mcyE copy numbers according to the regression equations of the external standards that gave the highest (Anabaena 202A1 and Microcystis PCC7941) and lowest (Anabaena 315 and Microcystis PCC7806) mcyE copy numbers (FIGS. 9A and B).

Amplification efficiencies, e (e=10^{-1/S}−1, s=slope of the linear regression), of the Anabaena and Microcystis mcyE QRT-PCR with standard strains were calculated as a function of known mcyE copy numbers and with those of Lake Tuusulanjärvi DNA samples as a function of different dilutions of the samples.

In order to determine melting temperatures for the amplification products of the standard strains and of the lake water samples, temperature was raised after QRT-PCR from 65° C. to 95° C. and fluorescence was detected continuously. Characteristic melting temperatures of the mcyE QRT-PCR products were determined with LightCycler software (version 3.5).

Microcystin analysis of the strains and lake water samples. Dry weight of the Anabaena, Microcystis, Planktothrix and Nostoc strains was measured and microcystin was extracted by sonication as detailed previously (Repka et al., 2001). Microcystin concentration of the strains was analyzed with an Agilent 1100 Series high performance liquid chromatograph with a diode array detector and Luna 5 μm C18 column (150×2 mm, Phenomenex). A mobile phase was 10 mM ammonium acetate and acetonitrile. During 6 to 40 minutes, concentration of acetonitrile increased from 24% to 60%. Flow rate was 0.2 ml min⁻¹ at 40° C., injection volume 20 μl, and detection at 238 nm. Purified microcystin-LR was used as a standard and microcystins were identified by their UV spectra and retention times.

Total microcystin of the lake water samples was extracted from 5 ml of lake water using tip sonicator for 5 min (Braun Labsonic-U). Prior measuring microcystin concentration with EnviroGard® microcystins plate kit (Strategic Diagnostics Inc.) and plate spectrophotometer (Labsystems iEMS reader MF) samples were filtered through 0.2 μm Puradisc™ filters (Whatman) to remove the particles.

Statistical analysis. Spearman correlation coefficients between microcystin concentration (μg 1⁻¹), mcyE copy numbers (copies ml⁻¹), and Anabaena as well as Microcystis cell numbers (cells ml⁻¹) of lake water samples were calculated with SAS® statistical software for Windows (SAS Institute Inc.).

Specificity of the primers. The mcyE gene primers (Table 6) were both genus and mcyE gene specific, since a single amplification product was observed when genomic DNA of microcystin producing Anabaena or Microcystis strain was used as a template in PCR with Anabaena or Microcystis genus specific primers (Table 7).

Detection range of mcyE copy numbers. The QRT-PCR was log-linear from 6.6×10² to 6.6×10⁵ mcyE copies in a reaction when the genomic DNAs of the standard strains Anabaena 90, Anabaena 202A1, Microcystis GL 260735 or Microcystis PCC 7941 were used as a template and from 6.6×10² to 6.6×10⁶ when those of standard strains Anabaena 315 or Microcystis PCC 7806 were used (FIGS. 9A and B). The lowest reliable mcyE copy numbers in Lake Tuusulanjärvi were 42, 84, 33, and 63 copies ml⁻¹ when calculated with the regression equations of the standards Anabaena 315, Anabaena 202A1, Microcystis 7806, and Microcystis 7941. In Lake Hiidenvesi the lowest reliable mcyE copy numbers were ten times higher than in Lake Tuusulanjärvi, 420, 840, 330, and 630 copies ml⁻¹ when calculated with the same standards, respectively. One ng of genomic DNA of Anabaena and Microcystis standard strains contained 1.76×10⁵ and 1.94×10⁵ mcyE copies. The purity of these DNAs varied from 1.8 to 1.9.

The mcyE copy numbers of lake water. Microcystis mcyE copy numbers in Lake Tuusulanjärvi were 11 to 91 times more abundant than those of Anabaena mcyE copy numbers calculated as a ratio of the average mcyE copy numbers obtained with Anabaena 315, Anabaena 202A1, Microcystis PCC 7941 and Microcystis PCC 7806 standards (FIG. 10). Microcystis mcyE copy numbers were also more abundant than those of Anabaena in the Basin of Kiihkelyksenselkä of Lake Hiidenvesi (FIG. 11). In the Basins of Nummelanselkä and in Mustionselkä Microcystis and Anabaena mcyE copy numbers were quite similar (FIG. 11). In the Basin of Kirkkojärvi both Microcystis and Anabaena mcyE copy numbers were below the detection limits determined with the standards (FIG. 11). In Lake Hiidenvesi (FIG. 11) the average mcyE copy numbers of Anabaena and Microcystis as well as microcystin concentrations were lower than in Lake Tuusulanjärvi (FIG. 11). Microcystin concentration had a statistically significant positive correlation with Microcystis mcyE copy numbers of all studied samples within the mcyE copy number detection range determined with the standards (Table 8).

Amplification efficiency. With Lake Tuusulanjärvi water samples the Microcystis mcyE QRT-PCR amplification efficiencies (0.78-0.99, Table 4) were similar to the amplification efficiencies of the Microcystis standards (0.86-0.94, Table 4). However, Anabaena mcyE QRT-PCR amplification efficiencies with Lake Tuusulanjärvi water samples (1.14 to 2.36, Table 4) were unrealistic high compared to the amplification efficiencies of the Anabaena standard strains (0.96-0.99, Table 9).

Melting curve analysis. Characteristic melting temperatures of the mcyE QRT-PCR products (247 bp) of the three Anabaena (average=79.6° C., CV=0.4%, n=38, Table 5) and three Microcystis (average=81.5° C., CV=0.2%, n=38, Table 5) standard strains corresponded to the melting temperatures of Anabaena (average=79.3° C., CV=0.3%, n=58) and Microcystis (average=81.7° C., CV=0.2%, n=63) mcyE QRT-PCR products amplified with lake water samples (data not shown). The 1.9° C. difference in the average characteristic melting temperatures was due to over 40 nucleotide difference between Anabaena and Microcystis mcyE sequences.

Primer dimers were detected in Anabaena and in Microcystis mcyE QRT-PCR with negative controls and in Anabaena mcyE QRT-PCR with lake water samples that had low template DNA concentration, although hot start Taq DNA polymerase provided by the manufacturer of the kit was used. The error caused by the primer dimers was avoided by measuring fluorescence of Anabaena and Microcystis mcyE QRT-PCR amplification at higher temperature (77° C., 78° C., respectively) than the melting temperature of the primer dimers.

Microcystin concentration and cyanobacterial cell density of lake water. Microcystin concentrations as well as Anabaena and Microcyptis cell densities were highest in Lake Tuusulanjärvi on July and started to decrease thereafter (FIGS. 10 and 12). In Lake Hiidenvesi microcystin concentrations and cell densities were lower than those in Lake Tuusulanjärvi (FIGS. 11 and 13). According to microscope analysis, Microcystis cells were more abundant than Anabaena cells in Lake Tuusulanjärvi whereas Microcystis cells were observed only occasionally in Like Hiidenvesi. Anabaena was the most dominant genus in the Basins of Kirkkojärvi and Mustionselkä of Lake Hiidenvesi whereas Aphanizomenon was the most dominant genus in the Basins of Nummelanselkä and Kiihkelyksenselkä of Lake Hiidenvesi as well as in the Lake Tuusulanjärvi.

TABLE 6
Primers used in this study.

| Primer | Sequence (5′ to 3′) | |
|---|---|---|
| mcyE-F2 | GAA ATT TGT GTA GAA GGT GC * | (SEQ ID |
| | | NO 64) |
| AnamcyE-12R | CAA TCT CGG TAT AGC GGC | (SEQ ID |
| | | NO 65) |
| MicmcyE-R8 | CAA TGG GAG CAT AAC GAG | (SEQ ID |
| | | NO 66) |

* Forward primer, mcyE-F2, used in this study, was described in Example 2

TABLE 7
Specificity of Anabaena (mcyE-F2, AnamcyE-12R) and Microcystis (mcyE-F2,
MicmcyE-R8) microcystin synthetase E (mcyE) primers was studied
using Anabaena, Microcystis, Planktothrix, and Nostoc strains. Presence (+) or
absence (−) of the mcyE product. Microcystin (MC) production (+) or
lack of production (−). Accession numbers indicate mcyE sequences
available in GenBank. Culture collections: PCC, Pasteur Culture Collection,
Paris, France; NIVA-CYA, Norwegian Institute for Water Research,
Oslo, Norway; NIES, National Institute for Environmental Studies, Tsukuba, Japan.

| Genus | | Anabaena | Microcystis mcyE | Accession | |
|---|---|---|---|---|---|
| Strain | MC | mcyE primers | primers | No | Reference |
| Anabaena | | | | | |
| 66A | + | + | − | XX | 47, b |
| 90 | + | + | − | AJ536156 | 47, a |
| 202A1 | + | + | − | XX | 47, b |
| 202A2/41 | + | + | − | XX | 47, b |
| NIVA-CYA83/1 | + | + | − | XX | 47, b |
| 315 | + | + | − | XX | b |
| 318 | + | + | − | XX | b |
| 86 | − | − | − | | 46 |
| 123 | − | − | − | | 46 |
| 14 | − | − | − | | 46 |
| PCC 6309 | − | − | − | | 43 |
| PCC 7108 | − | − | − | | 43 |
| PCC 73105 | − | − | − | | 43 |
| PCC 9208 | − | − | − | | 43 |
| Microcystis | | | | | |
| 98 | + | − | + | XX | 47, b |
| 205 | + | − | + | XX | 47, b |
| GL 260735 | + | − | + | XX | 55, b |
| GL 280646 | + | − | + | XX | 55, b |
| IZANCYA5 | + | − | + | XX | 53, b |
| IZANCYA25 | + | − | | XX | 53, b |
| NIES102 | + | − | | XX | 29, b |
| NIES A89 | + | − | + | XX | 29, b |
| PCC 7941 | + | − | + | XX | 43, b |
| PCC 7806 | + | − | + | AF183408 | 43, 51 |
| 130 | − | − | − | | 44 |
| 269 | − | − | − | | 44 |
| GL 060916 | − | − | − | | 55 |
| Planktothrix | | | | | |
| 49 | + | − | − | XX | 47, b |
| 97 | + | − | − | XX | 47, b |
| 213 | + | − | − | | 47 |
| NIVA-CYA 126 | + | − | − | AJ441056 | 9, 47 |
| NIYA-CYA 127 | + | − | − | XX | 47, b |
| NIVA-CYA 128/R | + | − | − | XX | 47, b |
| 45 | − | − | − | | 44 |
| PCC 6304 | − | − | − | | 43 |
| Nostoc | | | | | |
| 152 | + | − | − | XX | 48, b |

a Example 1
b Example 2

(9) Christiansen et al. 2003, (29) Lyra et al., 2001, (43) Rippka and Herdman, 1992, (44) Rouhiainen et al. 1995, (46) Sivonen and Jones, 1999, (47) Sivonen et al. 1989, (48) Sivonen et al. 1995, (53) Vasconcelos et al., 1995, (55) Vezie et al. 1998,

TABLE 8
Spearman correlation coefficients between microcystin concentration (μg 1⁻¹) and
microcystin synthetase E (mcyE) copy numbers (copies ml⁻¹) calculated using different
standards (Anabaena 202A1, Anabaena 315, Microcystis PCC 7806 and Microcystis
PCC7941) and cell numbers (cells ml⁻¹) in Lake Tuusulanjärvi and Lake Hiidenvesi. Sum of
Anabaena and Microcystis mcyE copy numbers was counted by adding the average copy
numbers calculated using the two Anabaena and Microcystis standards. Number inside the
parenthesis shows the number of samples used to calculate the spearman correlation.

| | | | | | Sum of | | | |
|---|---|---|---|---|---|---|---|---|
| | | | | | Anabaena and | | | Microcystis |
| | Anabaena | | Microcystis | | Microcystis | Microcystis | Anabaena | Anabaena |
| | McyE | | mcyE | | mcyE | cells | cells | cells |
| Lake water | 202 | 315 | PCC | PCC | | | | |
| samples | A1 | | 7806 | 7941 | | | | |
| All samples | | | 0.57* | 0.57* | 0.52, p= 0.10 | | | |
| | (11) | (11) | (15) | (15) | (11) | (21) | (21) | (21) |
| Lake | 1*** | 1*** | | | | | | 0.86 * |
| Tuusulanjärvi | (5) | (5) | (6) | (6) | (5) | (7) | (7) | (7) |
| Lake | (6) | (6) | (9) | (9) | (6) | (14) | (14) | (14) |
| Hiidenvesi | | | | | | | | |

*p < 0.5,
**p < 0.1,
***p < 0.01

TABLE 9
Anabaena and Microcystis mcyE QRT-PCR amplification efficiencies, e (e = 10^{-1/S}−
1, S = slope of linear regression equation), of the external standard strains calculated as a
function of mcyE copy numbers and those of Lake Tuusulanjärvi water samples calculated as
a function of different dilutions of the samples. r² denotes coefficient of determination.

| Strain or | Amplification | | | mcyE copy numbers or |
|---|---|---|---|---|
| Sampling date | efficiency | S | r² | Dilution factors |
| Microcystis | | | | |
| GL 260735 | 0.86 | −3.71 | 1 | 6.6 × 10², 6.6 × 10³, 6.6 × 10⁴, 6.6 × 10⁵ |
| PCC 7806 | 0.92 | −3.53 | 1 | 6.6 × 10², 6.6 × 10³, 6.6 × 10⁴, 6.6 × 10⁵, 6.6 × 10⁶ |
| PCC 7941 | 0.94 | −3.47 | 1 | 6.6 × 10², 6.6 × 10³, 6.6 × 10⁴, 6.6 × 10⁵ |
| 12-Jul | 0.95 | −3.46 | 1 | 1, 0.1, 0.05, 0.01, 0.005 |
| 2-Aug | 0.97 | −3.39 | 1 | 1, 0.1 |
| 23-Aug | 0.99 | −3.34 | 1 | 1, 0.1 |
| 7-Sep | 0.80 | −3.92 | 1 | 1, 0.1 |
| 20-Sep | 0.78 | −3.99 | 1 | 1, 0.1 |
| 6-Oct | 0.88 | −3.66 | 1 | 1, 0.1 |
| Anabaena | | | | |
| 90 | 0.96 | −3.41 | 1 | 6.6 × 10², 6.6 × 10³, 6.6 × 10⁴, 6.6 × 10⁵ |
| 315 | 0.99 | −3.34 | 1 | 6.6 × 10², 6.6 × 10³, 6.6 × 10⁴, 6.6 × 10⁵, 6.6 × 10⁶ |
| 202A1 | 0.98 | −3.36 | 1 | 6.6 × 10², 6.6 × 10³, 6.6 × 10⁴, 6.6 × 10⁵ |
| 12-Jul | 1.32 | −2.74 | 1 | 1, 0.1, 0.05 |
| 2-Aug | 1.14 | −3.02 | 1 | 1, 0.1, 0.05 |
| 23-Aug | 1.32 | −2.74 | 1 | 1, 0.1 |
| 7-Sep | 2.36 | −1.90 | 0.98 | 1, 0.1, 0.05 |

TABLE 10
Characteristic melting temperatures (Tₘ ± CV %) of the microcystin synthetase E
quantitative real-time PCR amplification products (247 bp) obtained using LightCycler
melting curve analysis. Nucleotide differences were calculated for the 209 bp long
sequence between the primer annealing sites. Number of samples is denoted by n.

| | | | Nucleotide differences | | | | | |
|---|---|---|---|---|---|---|---|---|
| | | | Anabaena | | | Microcystis | | |
| | | | | | 202 | GL 26 | PCC | PCC |
| Strain | Tₘ ± CV % | n | 90 | 315 | A1 | 0735 | 7806 | 7941 |
| Anabaena | | | | | | | | |
| 90 | 79.7 ± 0.2 | 12 | | | | | | |
| 315 | 79.3 ± 0.4 | 14 | 0 | | | | | |
| 202A1 | 79.7 ± 0.2 | 12 | 1 | 1 | | | | |
| Microcystis | | | | | | | | |
| GL 260735 | 81.3 ± 0.2 | 12 | 45 | 45 | 46 | | | |
| PCC 7806 | 81.5 ± 0.2 | 15 | 47 | 47 | 48 | 2 | | |
| PCC 7941 | 81.5 ± 0.1 | 11 | 47 | 47 | 48 | 2 | 1 | |

### Example 4

We were interested in the mcyD gene region as part of an evolutionary study on microcystin synthetase genes from different genera of cyanobacteria.

The McyD gene is involved in the formation of the Adda amino acid and this amino acid along with D-glutamate is critical to microcystin toxicity (Goldberg, J., Huang, H-B., Kwon, is Y-G., Greengard, P., Nairn, A. C. et al. Three-dimensional structure of the catalytic subunit of protein serine/threonine phosphatase-1. Nature 376, 745-753 (1995). The Adda amino acid is proposed to be assembled by McyG, McyD and mcyE (Tillett, D. et al. Structural organization of microcystin biosynthesis in Microcystis aeruginosa PCC7806: an integrated peptide-polyketide synthetase system. Chem. Biol. 7, 753-764 (2000). The mcyD gene region we sequenced encodes parts of a beta-ketoacyl synthase and a acyltransferase domain (Tillett et al. 2000). The region we looked at is specifically involved in one round of chain elongation of the growing Adda amino acid (Tillett et al. 2000).

The 818 bp region of the mcyD gene was amplified with the mcyDF (5′-gatccgattgaattagaaag-3) and mcyDR (5′-gtattccccaagattgcc-31) primers. PCR reactions for the mcyD PCR products were performed in a 20 ml final volume containing 1 ml of DNA, 1×DynaZyme II PCR buffer, 250 mM of each deoxynucleotide, 0.5 mM of both PCR primers, and 0.5 U of DynaZyme II DNA polymerase (Finnzymes, Espoo, Finland). The following thermocycle protocol was used: 95° C., 3 min; 30×(94° C., 30 sec; 56° C., 30 sec; 72° C., 1 min); 72° C., 10 min. Sequencing of the mcyD PCR products was performed by Genome Express (France).

### Example 5

Oligonucleotides for Detection and Identification of Toxic Cyanobacteria

Materials and Methods

All chemicals and solvents were purchased from Sigma-Aldrich (Italy) and used without further purification. Oligonucleotides were purchased from Interactiva Biotechnologie GmbH (Germany).

DNA Samples

The samples used to validate the probes were Anabaena 202A1, Microcystis 205, Planktothrix 49, Nostoc 152 and the environmental samples 0TU35 (>10 um fraction) and 0TU33 (bloom sample).

Ligation Probe Design

For Ligation Detection Reaction, we designed specific probes for the mcyE sequences of five different groups. These groups were identified using a phylogenetic tree obtained from the ARB software, version Beta 011107.

ARB (www.arb-home.de) is a UNIX-based program for aligning a large number of DNA sequences and for constructing phylogenetic trees according to a central database of processed sequences.

The mcyE sequences were aligned using CLUSTAL W (Thompson et al., 1994) and internal ARB algorithms. The phylogenetic tree was constructed using the neighbor-joining (NJ) algorithm (Saitou and Nei, 1987). The groups are the following: Anabaena, Microcystis, Nodularia, Nostoc, Oscillatoria/Planktothrix (OP).

From the sequence alignment a “group-specific” consensus sequence was obtained with a cutoff percentage of 95%. This value is compared with the frequency of the residues found at each alignment position. If the residue at a given position occurred at a lower frequency than the cutoff percentage, an IUPAC ambiguous symbol was displayed in the consensus sequence.

Then, group-specific probe design was obtained using a tool on ARB database named “Probe design”.

All oligonucleotides were designed to have a melting temperature (Tₘ) between 64 and 68° C.

Discriminating probes were purchased with a Cy3 label at their 5′ terminal position and common probes with a phosphate in the same position.

Universal Array Preparation

Microarrays were prepared using CodeLink™ slides (Amersham Biosciences), designed to covalently immobilize NH₂-modified oligonucleotides.

5′ amino-modified Zip Code oligonucleotides, carrying an additional poly(dA)₁₀ tail at their 5′ end, were diluted to 25 μM in 100 mM phosphate buffer (pH 8.5). Spotting was performed using a non contact piezo driven dispensing system (Nanoplotter, GeSim, Germany). Printed slides were processed according to the manufacturer's protocols.

Quality control of printed surfaces was performed by sampling one slide from each deposition batch. The printed slide was hybridized with 1 μM 5′ Cy3 labeled poly(dT)₁₀ in a solution containing 5×SSC and 0.1 mg/ml salmon sperm DNA at RT for 2 h, then washed for 15 min in 1×SSC. The fluorescent signal was controlled by laser scanning following procedures described in “Array hybridization, detection and data analysis”.

PCR Amplifications from DNA Samples.

Ligation Detection Reaction.

Ligation Detection Reaction was carried out in a final volume of 20 μl containing 20 mM Tris-HCl (pH 7.5), 20 mM KCl, 10 mM MgCl₂, 0.1% NP40, 0.01 mM ATP, 1 mM DTT, 2 pmol of each discriminating probe, 2 pmol of each common probe and 100 fmol of purified PCR products. The reaction mixture was preheated for 2 min at 94° C. and spinned in a microcentrifuge for 1 min; then 1 ul of 4 U/ul Pfu DNA ligase (Stratagene, La Jolla, Calif.) was added. Alternatively, 0.5 ul of 50 U/ul Tth DNA ligase (ABgene) was used.

The LDR was cycled for 30 rounds of 90° C. for 30 sec and 60° C. for 4 min in the GeneAmp PCR system 9700 thermal cycler (Applied Biosystems, California).

Array Hybridization, Detection and Data Analysis.

In a 0.5-ml microcentrifuge tube, the LDR mix (20 μl) was diluted to obtain 65 μl of hybridization mixture containing 5×SSC and 0.1 mg/ml salmon sperm DNA. The mix, after heating at 94° C. for 2 min and chilling on ice, was applied onto the slide under a hybridization chamber.

Hybridization was carried out in the dark at 65° C. for two hours in a temperature-controlled water bath. After hybridization, the microarray was washed at 65° C. for 15 min in pre-warmed 1×SSC, 0.1% SDS. Finally, the slide was spinned at 80 g for 3 min.

The fluorescent signals were acquired at 5 μm resolution using a ScanArray® 4000 laser scanning system (PerkinElmer Life Sciences) with green laser for Cy3 dye (λₑₓ 543 nm/λₑₘ 570 nm). Both the laser and the photomultiplier (PMT) tube power were set at 70-95%. To quantitate the fluorescent intensity of the spots we used the QuantArray Quantitative Microarray Analysis software (Perkin Elmer Life Sciences).

Recently, we have presented a Universal DNA Array approach to discriminate some groups of bacteria (Busti et al., 2002). This procedure, based on the discriminative properties of the DNA ligation reaction, requires the design of two probes specific for each target sequence, as described by Barany and co-workers (1999). One oligonucleotide brings a fluorescent label and the other a unique sequence named complementary Zip Code (cZip Code). Ligated fragments, obtained in presence of a proper template by the action of a DNA ligase, are addressed to the location on the microarray where the Zip Code sequence has been spotted. Such an array is therefore “Universal” being unrelated to a specific molecular analysis.

Here we present the Universal DNA Array approach applied to the detection of cyanobacterial mcyE gene diversity.

Ligation Probes Design

We used the ARB software to perform the sequence alignment of cyanobacterial mcyE sequences. These sequences were aligned and clustered according to their phylogenetic lineages so that 5 “group-specific” consensus sequences were yielded: Anabaena, Microcystis, Nodularia, Nostoc, Oscillatoria/Planktothrix (OP) (FIG. 14). Then, “group-specific” probes were designed using a tool on ARB database named “Probe design”. Among this set of probes, we selected discriminating probes with 3′ position unique to each group in order to obtain ligase discrimination. As a matter of fact, after hybridization of a discriminating probe and a common probe to the target sequence, ligation occurs only if there is perfect complementarity at the junction between the two oligos. Common probes were designed immediately 3′ to the discriminating oligo from the group-specific consensus.

All the selected probes are described in FIG. 20. We selected one probe pair for each group of interest, except for the Oscillatonia/Planktothrix group.

FIG. 15 shows the alignment of the “group-specific” consensus sequences and the relative discriminating probes.

Zip Codes Assignment and Quality Control of the Universal Array

We randomly selected 6 Zip code sequences from those described by Chen and co-workers, 2000. Each Zip code was randomly assigned to a single cyanobacterial group. Each common probe was synthesized to have the complementary Zip code (cZip code) affixed to its 3′ end (FIG. 20). No significant self-annealing of the common probe-cZip sequences was detected by computer analysis (data not shown).

The Zip codes were deposited using a non contact deposition system. The deposition scheme is shown in FIG. 17. In order to verify the deposition quality of the Zip Code oligonucleotides on the slides, we performed hybridisations with Cy3 labelled poly(dT) complementary to the poly(da)₁₀ sequence of each Zip Code. Every controlled slide revealed intense fluorescent signals corresponding the spotted oligonucleotides, as shown in FIG. 17.

This result indicated a rather uniform deposition of the oligos on the Universal Array.

LRD detection onto Universal Array

1) Probes Specificity

The specificity of the probes for mcyE cyanobacterial groups was tested using PCR amplified fragment of this gene coming either from pure strains or from environmental samples, as indicated in Materials and Methods.

LDRs were conducted in the presence of the PCR product of each single sample as template and in the presence of all the probes (discriminating probes and common probes).

A negative control of the entire process was performed using double distilled water instead of genomic. DNA as PCR substrate. After standard cycling, ten microliters of the reaction mixture were used in the LDR. Following hybridisation on the universal chip, no signal was detected even setting PMT and laser to 95% of their power (data not shown).

In the presence of the proper DNA template, the Universal Array behaved as expected: only group-specific spots showed positive signal. The results are showed in FIG. 18.

2) Probe Sensitivity

In order to establish the detection limit of the method, we performed the Ligation Detection Reaction starting from 50, 5 and 1 fmol of three different PCR products as substrates. The detected signals progressively decrease and three visible signals were detected up to 1 finol of the PCR products. No signals were detected using 0.5 fmol of the substrates even setting PMT and laser to 95% of their power (data not shown).

### Example 6

Molecular Analysis of Cyanobacterial Diversity by Microarrays on “PCR-Amplified” 16 rRNA Gene

All chemicals and solvents were purchased from Sigma-Aldrich (Italy) and used without further purification. Oligonucleotides were purchased from Interactiva Biotechnologie GmbH (Germany).

DNA Samples

The samples used to validate the probes included axenic strains kept in the authors' culture collections, strains isolated from European lakes and a reservoir during this study, and clones of environmental DNA libraries obtained from Lake Esch-sur-Sûre (Luxembourg) and Lake Tuusulanjärvi (Finland). The 16S rRNA gene of the cultured strains and clones was sequenced (unpublished data). In addition, the array was tested with an environmental DNA sample (Lake Tuusulanjärvi), which was isolated with the hot-phenol method. To verify the microarray results, the same environmental sample was analyzed with DGGE and cloning of the 16S rRNA gene.

Ligation Probe Design

For Ligation Detection Reaction, we designed specific probes for the 16S rRNA gene sequences of different cyanobacterial groups. These groups were identified using a cyanobacterial 16S rRNA gene tree obtained from the ARB software, version Beta 011107.

ARB (www.arb-home.de) is a UNI-based program for aligning a large number of 16S rRNA gene sequences and for constructing phylogenetic trees according to a central database of processed sequences. ARB cyanobacterial 16S rDNA database we used contained 281 sequences from public databases and 57 from this study, in addition to the outgroup Escherichia coli. All these sequences were longer than 1400 bp, except the two sequences of Antarctic Phormidium (about 1350 bp) and 21 (out of 42) sequences of Prochlorococcus marinus (about 1250 bp). All sequences were aligned with CLUSTAL W (24) and ARB. The phylogenetic tree was constructed using the neighbor-joining (NJ) algorithm (Saitou and Nei, 1987). As shown in FIG. 25, the selected cyanobacterial groups are the following: Anabaena/Aphanizomenon, Calothrix, Cylindrospermopsis, Cylindrospermum, Gloeothece, Halotolerants, Leptolyngbya, Palau Lyngbya, Microcystis, Nodularia, Nostoc, Oscillatoria/Planktothrix, Antarctic Phormidium, Prochlorococcus, Spirulina, Synechococcus, Synechocystis, Trichodesmium, Woronichinia.

From the sequence alignment a “group-specific” consensus sequence was obtained with a cutoff percentage of 75%. This value is compared with the frequency of the residues found at each alignment position. If the residue at a given position occurred at a lower frequency than the cutoff percentage, an IUPAC ambiguous symbol was displayed in the consensus sequence.

Then, the 19 group consensus sequences were imported in GCG Omiga 2.0 (Oxford Molecular Ltd.) for group-specific probe design. The specificity of each probe pair (discriminating probe and common probe) was controlled on the entire bacterial 16S rDNA ARB database. All oligonucleotides were designed to have a melting temperature (Tₘ) between 64 and 68° C.

Discriminating probes were purchased with a Cy3 label at their 5′ terminal position and common probes with a phosphate in the same position.

Universal Array Preparation

Microarrays were prepared using CodeLink™ slides (Amersham), designed to covalently immobilize NH₂-modified oligonucleotides.

5′ amino-modified Zip Code oligonucleotides, carrying an additional poly(dA)₁₀ tail at their 5′ end, were diluted to 25 μM in 100 mM phosphate buffer (pH 8.5). Spotting was performed using a contact dispensing system MicroGrid II (BioRobotics). Printed slides were processed according to the manufacturer's protocols. 8 subarrays per slide were generated.

Quality control of printed surfaces was performed by sampling one slide from each deposition batch. The printed slide was hybridized with 1 μM 5′ Cy3 labeled poly(dT)₁₀ in a solution containing 5×SSC and 0.1 mg/ml salmon sperm DNA at RT for 2 h, then washed for 15 min in 1×SSC. The fluorescent signal was controlled by laser scanning following procedures described in “Array hybridization, detection and data analysis”.

PCR Amplifications from DNA Samples.

The DNA region coding for 16S ribosomal RNA was amplified with a universal primer 16SF27 (5′AGAGMTIGATCMTGGCTCAG 3′) (Edwards et al., 1989) and a cyanobacterial specific primer 23S30R (5′CCTCGCCTCTGTGTGCCTAGGT3) (Lepère et al., 2000) which permitted the amplification of a ca 2000 bp fragment.

PCR amplifications were performed in a GeneAmp PCR system 9700 thermal cycler (Applied Biosystem, California). The reaction mixtures include 500 nM each primer, 200 μM each dNTP, 10 mM Tris-HCl (pH 8.8), 1.5 mM MgC₂, 50 mM KCl, 0.1% (wt/vol) Triton X-100, 1 U of DynaZyme DNA polymerase (Finnzymes OY, Espoo, Finland) and 5-8 ng of genomic DNA, in a final volume of 50 μl. Prior to amplification, DNA was denatured for 5 min at 95° C. Amplification consisted of 30 cycles of 94° C. for 45 s, 57° C. for 45 s and 72° C. for 2 min. After the cycles, an extension step (10 min at 72° C.) was performed.

The PCR products were purified by GFX PCR DNA purification kit (Amersham Biosciences, Piscataway-NJ), eluted in 50 μl of autoclaved water and quantified by the BioAnalyzer 2100 (Agilent Technologies).

Ligation Detection Reaction

Ligation Detection Reaction was carried out in a final volume of 20 μl containing 20 mM Tris-HCl (pH 7.5), 20 mM KCl, 10 mM MgCl₂, 0.1% NP40, 0.01 mM ATP, 1 mM DTT, 250 fmol of each discriminating probe, 250 fmol of each common probe, 10 fmol of the hybridization control and 25 fmol of purified PCR products. The reaction mixture was preheated for 2 min at 94° C. and spinned in a microcentrifuge for 1 min; then 1 ul of 4 U/ul Pfu DNA ligase (Stratagene, La Jolla, Calif.) was added. The LDR was cycled for 30 rounds of 90° C. for 30 sec and 60° C. for 4 min in the GeneAmp PCR system 9700 thermal cycler (Applied Biosystems, California).

Array Hybridization, Detection and Data Analysis.

In a 0.5-ml microcentrifuge tube, the IDR mix (20 μl) was diluted to obtain 65 μl of hybridization mixture containing 5×SSC and 0.1 mg/ml salmon sperm DNA. The mix, after heating at 94° C. for 2 min and chilling on ice, was applied onto the slide in the Press-To-Seal Silicone Isolators 1.0×9 mm (Schleicher & Schuell).

Hybridization was carried out in a hybridization chamber in the dark at 65° C. for two hours in a temperature-controlled water bath. After hybridization, the microarray was washed at 65° C. for 15 min in pre-warmed 1×SSC, 0.1% SDS. Finally, the slide was spinned at 80 g for 3 min.

The fluorescent signals were acquired at 5 μm resolution using a ScanArray® 4000 laser scanning system (PerkinElmer Life Sciences) with green laser for Cy3 dye (λ_{wx} 543 nm/λₑₘ 570 nm). Both the laser and the photomultiplier (PMT) tube power were set at 70-95%.

To quantify the fluorescent intensity of the spots we used the QuantArray Quantitative Microarray Analysis software (Perkin Elmer Life Sciences).

When statistical analyses were performed, we included the fluorescent intensity values obtained from replicated spots (four replicates spot for each group, eight replicates spot for the universal) and replicates experiments sets (three LDR-universal array experiments).

Sequence Analysis of Cyanobacterial 16S rDNA and Ligation Probes Design

We used the ARB software to perform the sequence alignment of cyanobacterial 16S rDNA. The ARB database we used contained 281 cyanobacterial sequences from public databases and 57 from this study. These sequences were aligned and clustered according to their phylogenetic lineages so that 19 “group-specific” consensus sequences were yielded (FIG. 25).

Then, the 19 group consensi were imported in GCG Omiga 2.0 (Oxford Molecular Ltd.). The Omiga software is a graphically oriented package that permits the identification of “group-specific” nucleotide polymorphisms. Thus, the probes were designed complementary to polymorphic regions on the basis of a final alignment among group-specific consensi. The selection process consisted in several steps. Firstly, we considered the ligase reaction features. As shown in FIG. 26, after hybridization of a discriminating probe and a common probe to the target sequence, ligation occurs only if there is perfect complementarity at the junction between the two oligos. For this reason, to obtain ligase discrimination, we selected discriminating probes with 3′ position unique to each group. Common probes were designed immediately 3′ to the discriminating oligo from the group-specific consensus.

Secondly, among this set of probes, we selected only those pairs of probes, which differed from all representatives of the other groups at least for the 3′ terminal position of the discriminating probes, but which were invariant in all members of their group. Examples of probe design procedure are shown in FIG. 27.

Finally, in order to discard potentially a specific probe pairs, we analyzed each probe pair (discriminating probe and common probe) using a tool on ARB database, which permit to verify probes against all the bacterial 16S rRNA gene sequences. Initially, we considered 60 group specific probe pairs, but only 21 of these have been chosen after the selection step described above.

All the selected probes are described in FIG. 32. When the consensus sequence contains a degenerate base, we included inosine during oligonucleotide synthesis at these degenerate positions.

Although DNA samples for some of the 19 selected groups (i.e. Gloeothece, Antarctic Phormidium, Prochlorococcus marinus, Trichodesmium) were not available because these cyanobacteria are not present in the lakes under scrutiny, all the ARB phylogenetic lineages have been considered in the experimental set-up to allow for future applications of this cyanobacterial microarray.

In order to have a positive control for the Ligation Detection Reaction, a universal probe pair, matching all the cyanobacteria, was designed and the corresponding Zip code was included in the Universal Array. As a positive control for the hybridisation reaction, a Cy3 labelled complementary Zip Code sequence was added in the hybridization mixture and the corresponding Zip code was included in the Universal Array.

Zip Codes Assignment and Quality Control of the Universal Array

We randomly selected 21 Zip code sequences from those described by Barany and coworkers and Chen and co-workers. Each Zip code was randomly assigned to a single cyanobacterial group, except Zip code1 which is the positive control for the hybridisation reaction.

Each common probe was synthesized to have the complementary Zip code (cZip code) affixed to its 3′ end (FIGS. 32 and 39). No significant self-annealing of the twenty common probe-cZip sequences was detected by computer analysis (data not shown).

The Zip codes were deposited using a contact deposition system generating 8 subarrays per slide. The deposition scheme is shown in FIG. 28. In order to verify the deposition quality of the Zip Code oligonucleotides on the slides, we performed hybridisations with Cy3 labelled poly(dt) complementary to the poly(da)₁₀ sequence of each Zip Code.

LDR Detection onto Universal Array of Cyanobacterial 16S rDNA Samples

1) Probes Specificity

The specificity of the probes for freshwater cyanobacterial groups was tested using PCR amplified 16S rRNA gene coming either from pure strains (both axenic and isolated in this study) or from cloned rDNA sequences. All pure strains used to validate the LDR probes are described in FIG. 33. The sequences obtained from the clones have been aligned in the ARB database with the sequences of pure cyanobacterial strains in order to define their phylogenetic group. The clones used are described in FIG. 34.

LDRs were conducted in the presence of the PCR product of each single strain or clone as template and in the presence of all the probes (discriminating probes and common probes).

A negative control of the entire process was performed using double distilled water instead of genomic DNA as PCR template. After standard cycling, ten microliters of the reaction mixture were used in the LDR. Following hybridisation on the Universal Array, no signal was detected even setting PMT and laser to 95% of their power (data not shown).

In the presence of the proper DNA template, the Universal Array behaved as expected: only group specific spots, universal spots and the spots corresponding to the hybridization control showed positive signal. Some of the results are shown in FIG. 29.

2) Probe Sensitivity

In order to establish the detection limit of the method and the correlation between signal intensity and template concentration, we performed Ligation Detection Reactions starting from 100 to 0,5 fmol of PCR products obtained from Planktothrix 1LT as substrates. The detected signals progressively decrease and a visible signal was detected up to 1 fmol of the PCR product. No signals were detected using 0.5 fmol of the substrates even setting PMT and laser to 95% of their power (data not shown). The linear correlation between signal intensity and template concentration is shown in FIG. 31.

3) Use of Artificial Mixes of PCR Products from Different Strains.

In order to determine the efficiency of the LDR method in presence of complex molecular targets, we used artificial mixes with unbalanced amounts of PCR products derived from the following cyanobacterial samples: Aphanizomenon sp. 202, Microcystis OBB 34S, Spirulina subsalsa PCC6313, Calotlrix sp. PCC7714, clone Woronichinia OES46. After separate PCR reactions, the amplified fragments were pooled in unbalanced LDR mixes using different ratios: 100:1, 50:1, 100:5, 50:5. In all these experiments Aphanizomenon sp. 202 and Microcystis OBB 34S were the more concentrated samples. Moreover, we mixed also 500 fmol of the amplicon derived from Microcystis OBB 34S with 5 fmol of the PCR fragment obtained from Woronichinia OES46 clone. After the hybridization of the LDR products onto the Universal Array, the signals related to the lower concentrated template were not detected in the LDR mixes with these ratio: 100:1 and 50:1. Only in presence of the LDR products obtained from the mixes with the ratio 100:5 and 50:5 all the expected signals are detected FIG. 29. The fluorescent intensity of the spots was quantified and the results are shown in FIG. 29. Furthermore, we compared also the results obtained using two LDR unbalanced mixes 100:1 (100 fmol of Microcystis OBB 34S and 1 fmol each of Spirina, Woronichinia and Calothrix), in one of which 8 U of Pfa DNA ligase was added, whereas the other was prepared using 4 U of the enzyme, as described in Materials and Methods. Hybridization signals of the lower concentrated substrates were detected only from the LDR product obtained using 8 U of Pfu DNA ligase instead of 4 U (FIG. 30).

LDR Detection onto Universal Array of 16S rDNA and mcyE from Environmental Samples

We made PCR amplification from genomic DNA using 16S cyanobacteria specific primers. The PCR conditions used are shown in FIG. 35. We made also PCR amplification from genomic DNA using mcyE gene primers. The ligation detection reaction was made under the same conditions by using an oligo mix containing both the probes for 16S rRNA gene and the probes for the mcyE gene as shown in FIG. 36. Finally the hybridization was carried on the same Universal Array where the 16S rRNA LDR product and, mcyE LDR product were detected

### Example 7

Microarray Platform for Toxic and Non-Toxic Detection in Cyanobacteria.

Materials and Methods.

All chemicals and solvents were purchased from Sigma-Aldrich (Italy) and used without further purification. Oligonucleotides were purchased from Interactiva Biotechnologie GmbH (Germany).

Ligation Probe Design

The mcyE probe design has been previously described in Example 5 in “Ligation probe design”. The 16S rRNA gene probe design has been previously described in Example 6 in “Ligation probe design”, but was added the probe design for a further cyanobacteria group: Snowella. The Snowella probe design was performed using the updated ARB database containing 281 sequences from public databases and 69 from this study (FIG. 25B). The updated database allowed to design specific probe for Aphanizomenon and Anabaena subgroups as shown in FIG. 25C. The probe design allows the detection of 20 toxic and non-toxic cyanobacteria groups.

Universal Array Preparation

Microarrays were prepared using CodeLink™ slides (Amersham), designed to covalently immobilize NH₂-modified oligonucleotides.

5′ amino-modified Zip Code oligonucleotides, carrying an additional poly(daA)₁₀ tail at their 5′ end, were diluted to 25 M in 100 mM phosphate buffer (pH 8.5). Spotting was performed using a contact dispensing system MicroGrid II (BioRobotics). Printed slides were processed according to the manufacturer's protocols. 8 subarrays per slide were generated.

The Universal array used for the detection of toxic and non-toxic cyanobacteria was designed to detect both the 16 rRNA and mcyE gene ligated probes. For this purpose the deposition scheme was improved as shown in FIG. 27B. We generated 8 subarray per slide. Each subarray is made of 208 spots including zipcodes for hybridization control, cyanobacterial universal probes, 16S rRNA gene specific probe, mcyE specific probe and empty spot as a negative control. Each specific zip code for the recognition of cyanobacteria universal probe, 16Ss RNA gene probe and mcyE gene probe is spotted in quadruplicate. The LDR positive control (zipcode no 63) is replicated 6 times, while the hybridization positive control (zipcode no 66) is replicated 8 times.

Quality control of printed surfaces was performed by sampling one slide from each deposition batch. The printed slide was hybridized with 1 μM 5′ Cy3 labeled poly(dt)₁₀ in a solution containing 5×SSC and 0.1 mg/ml salmon sperm DNA at RT for 2 h, then washed for 15 min in 1×SSC. The fluorescent signal was controlled by laser scanning following procedures described in “Array hybridization, detection and data analysis”.

PCR Amplification from DNA Samples

The PCR of mcyE gene and 16S rRNA gene were performed separately, using the conditions previously described in Examples 5 and 6 in “PCR amplification from DNA samples”.

Ligation Detection Reaction

The Ligation Detection Reaction for toxic and non-toxic cyanobacteria detection was done mixing together the PCR product of 16S rRNA and mcyE gene and the discrimination and common probe specific for both 16s rRNA and mcyE gene, FIG. 36.

Ligation Detection Reaction was carried out in a final volume of 20 μl containing 20 mM Tris-HCl (pH 7.5), 20 mM KCl, 10 mM MgCl₂, 0.1% NP40, 0.01 mM ATP, 1 mM DTT, 250 fmol of each discriminating probe, 250 fmol of each common probe, 10 fmol of the hybridization control and 25 fmol of purified PCR products. The reaction mixture was preheated for 2 min at 94° C. and spinned in a microcentrifuge for 1 min; then 1 ul of 4 U/ul Pfu DNA ligase (Stratagene, La Jolla, Calif.) was added. The LDR was cycled for 30 rounds of 90° C. for 30 sec and 60° C. for 4 min in the GeneAmp PCR system 9700 thermal cycler (Applied Biosystems, California).

Array Hybridization, Detection and Data Analysis

In a 0.5-ml microcentrifuge tube, the LDR mix (20 μl) was diluted to obtain 65 μl of hybridization mixture containing 5×SSC and 0.1 mg/ml salmon sperm DNA. The mix, after heating at 94° C. for 2 min and chilling on ice, was applied onto the slide in the Press-To-Seal Silicone Isolators 1.0×9 mm (Schleicher & Schuell).

Hybridization was carried out in a hybridization chamber in the dark at 65° C. for two hours in a temperature-controlled water bath. After hybridization, the microarray was washed at 65° C. for 15 min in pre-warmed 1×SSC, 0.1% SDS. Finally, the slide was spinned at 80 g for 3 min.

The fluorescent signals were acquired at 5 μAm resolution using a ScanArray® 4000 laser scanning system (PerkinElner Life Sciences) with green laser for Cy3 dye (λₑₓ 543 nm/λₑₘ 570 nm). Both the laser and the photomultiplier (PMT) tube power were set at 70-95%.

To quantify the fluorescent intensity of the spots we used the QuantArray Quantitative Microarray Analysis software (Perkin Elmer Life Sciences).

When statistical analyses were performed, we included the fluorescent intensity values obtained from replicated spots (four replicates spot for each group, eight replicates spot for the universal) and replicates experiments sets (three LDR-universal array experiments).

Zip Codes Assignment and Quality Control of the Universal Array

We randomly selected 33 Zip code sequences from those described by Chen and co-workers, 2000. Each Zip code was randomly assigned to a single cyanobacterial group. Each common probe, for both 16S rRNA and mcyE gene recognitin, was synthesized to have the complementary Zip code (cZip code) affixed to its 3′ end (FIGS. 20, 32 and 39). No significant self-annealing of the common probe-cZip sequences was detected by computer analysis (data not shown).

The Zip codes were deposited using a contact deposition system. The deposition scheme is shown in FIG. 27B. In order to verify the deposition quality of the Zip Code oligonucleotides on the slides, we performed hybridisations with Cy3 labelled poly(dT) complementary to the poly(da)₁₀ sequence of each Zip Code. Every controlled slide revealed intense fluorescent signals corresponding the spotted oligonucleotides, as shown in FIG. 27B. This result indicated a rather uniform deposition of the oligos on the Universal Array.

LDR Detection onto Universal Array of Cyanobacterial 16S rDNA and mcyE Samples

Probes Specificity

The specificity of the probes was tested using PCR amplified 16S rRNA and mcyE gene coming from pure strains (both axenic and isolated in this study.)

A negative control of the entire process was performed using double distilled water instead of genom DNA as PCR template. After standard cycling, ten microliters of the reaction mixture were used in the LDR. Following hybridisation on the Universal Array, no signal was detected even setting PMT and laser to 95% of their power (data not shown).

In the presence of the proper DNA template of both 16S rRNA and mcyE genes, the Universal Array functioned very well: only group specific spots, universal spots and the spots corresponding to the hybridization control showed positive signal. Some of the results are shown in FIG. 30B.

### REFERENCES

Altschul, S. F., T. L. Madden, A. A Schäffer, J. Zhang, Z. Zhang, W. Miller, and D. J. Lipman. 1997. Gapped BLAST and PSI-BLAST: a new generation of protein database search programs. Nucleic. Acids. Res. 25:3389-3402.
Amard, B. & Bertrand-Sarfati, J. Microfossils in 2000 Ma old cherty stromatolites of the Franceville Group, Gabon. Precambrian Res. 81′ 197-221 (1997).
Aparicio, J. F., Molnár, I., Schwecke, T., König, A., Haydock, S. F., Khaw, L. E., Staunton, J., Leadlay, P. F., 1996. Organization of the biosynthetic gene cluster for rapamycin in Streptomyces hygroscopicus: analysis of the enzymatic domains in the modular polyketide synthase. Gene 169, 9-16.
Baker, J. A., B. A Neilan, B. Entsch, and D. B. McKay. 2001. Identification of cyanobacteria and their toxigenicity in environmental samples by rapid molecular analysis. Environ. Toxicol. 16:472-482.
Baker, J. A., B. Entsch, B. A Neilan, and D. B. McKay. 2002. Monitoring changing toxigenicity of a cyanobacterial bloom by molecular methods. Appl. Environ. Microbiol. 68:6070-6076.
Becker, S., M. Fahrbach, P. Böger, and A. Ernst. 2002. Quantitative tracing, by Taq nuclease assays, of a Synechococcus ecotype in highly diversified natural population. Appl. Environ. Microbiol. 68:4486-4494.
Becker, S., P. Böger, R. Oehlmann, and A Ernst. 2000. PCR bias in ecological analysis: a case study for quantitative Taq nuclease assays in analyses of microbial communities. Appl. Environ. Microbiol. 66:4945-4953.
Boers, P., L. van Ballegooijen, and J. Uunk. 1991. Changes in phosphorus cycling in a shallow lake due to food web manipulations. Freshwater Biol. 25:9-20.
Brocks, J. J., Logan, G. A., Buick, R., & Summons, R. E. Archean molecular fossils and the early rise of eukaryotes. Science 285, 1033-1036 (1999).
Busti E, Bordoni R, Castiglioni B, Monciardini P, Sosio M, Donadio S, Consolandi C, Rossi Bernardi L, Battaglia C, De Bellis G. Bacterial discrimination by means of a universal array approach mediated by LDR (ligase detection reaction). BMC Microbiol 2:27 (2002).
Castenholz, R. W. 2001. Phylum BX. Cyanobacteria, oxygenic photosynthetic bacteria. p. 473-599. In D. R. Boone, R. W. Castenholz and G. M. Garrity (ed.), Bergey's manual of systematic bacteriology, 2^{nd} edition, vol. 1. Springer-Verlag.
Cavalier-Smith T. Origins of secondary metabolism. Ciba Found Symp. 171, 64-80 (1992).
Chen, J. et al. A microsphere-based assay for multiplexed single nucleotide polymorphism analysis using single base chain extension. Genome Res. 10, 549-557 (2000). A clear presentation of the concept of generic ‘tag’ sequences as applied to SNP genotyping.
Chorus I., and L. Mur. 1999. Preventative measures. p. 235-273. In I. Chorus and J. Bartram (ed.), Toxic cyanobacteria in water. E & FN Spon. London and New York.
Christiansen, G., Fastner, J., Erhard, M., Börner, T., Dittmann, E., 2003. Microcystin Biosynthesis in Planktothrix: Genes, Evolution, and Manipulation. J. Bacteriol. 185, 564-572.
DeMott, W. R. & Moxter, F. Foraging on cyanobacteria by copepods: responses to chemical defenses and resource abundance. Ecology 72, 1820-1834 (1991).
Dittmann, E., Neilan, B. A., Erhard, M., von Döhren, H., Börner, T., 1997. Insertional mutagenesis of a peptide synthetase gene that is responsible for hepatotoxin production in the cyanobacterium Microcystis aeruginosa PCC7806. Mol. Microbiol. 26, 779-787.
Dittmann, E. et al. Altered expression of two light-dependent genes in a microcystin-lacking mutant of Microcystis aeruginosa PCC 7806. Microbiol. 147, 3113-3119 (2001).
Du, L., Sanchez, C., Chen, M., Edwards, D. J., Shen, B., 2000. The biosynthetic gene cluster for the antitumor drug bleomycin from Streptomyces verticillus ATCC15003 supporting functional interactions between nonribosomal peptide synthesis and a polyketide synthase. Chem. Biol. 7, 623-642.
Duitman, E. H., Hamoen, L. W., Rembold, M., Venema, G., Seitz, H., Saenger, W., Bernhard, F., Reinhardt, R., Schmidt, M., Ullrich, C., Stein, T., Leenders, F., Vater, J., 1999. The mycosubtilin synthetase of Bacillus subtilis ATCC6633: A multifunctional hybrid between a peptide synthetase, an amino transferase, and a fatty acid synthase. Proc Natl Acad Sci USA 96, 13294-13299.
Edwards, U., Rogall, T., Blöcker, H., Emde, M. & Böttger, E. C. Isolation and direct complete nucleotide determination of entire genes. Characterization of a gene coding for 16S ribosomal RNA. Nucleic Acids Res. 17, 7843-7853 (1989).
Ekman-Ekebom, M., M. Kauppi, K Sivonen, M. Niemi, and L. Lepistö. 1992. Toxic cyanobacteria in some Finnish lakes. Environ. Toxicol. Wat. Qual. 7:201-213.
Falconer, I., J. Bartram, I. Chorus, T. Kuiper-Goodman, H. Utkilen, M. Burch, and G. A. Codd. 1999. Safe levels and safe practices. p. 155-178. In I. Chorus and J. Bartram (ed.), Toxic cyanobacteria in water. E & FN Spon. London and New York.
Fastner, J., Erhard, M., von Döhren H., 2001. Determination of oligopeptide diversity within a natural population of Microcystis spp. (cyanobacteria) by typing single colonies by matrix-assisted laser desorption ionization-time of flight mass spectrometry. Appl. Environ. Microbiol. 67, 5069-5076.
Felsenstein, J. PHYLIP (Phylogenetic Inference Package), version 3.5c. (Department of Genetics, Univ. Washington, Seattle, 1993).
Fujii, K, Harada, K, Suzuki, M., Kondo, F., Ikai, Y., Oka, H., Carmichael, W. W., Sivonen K, 1996. Occurrence of novel cyclic peptides together with microcystins from toxic cyanobacteria, Anabaena species. In: Yasumoto, T., Oshima, Y., Fukuyo, Y. (Eds.), Harmful and Toxic Algal Blooms. Intergovermental Oceanographic Commission of UNESCO, Paris, pp. 559-562.
Gerry, N. P. et al. Universal DNA mnicroarray method for multiplex detection of low abundance point mutations. J. Mol. Biol. 292, 251-262 (1999).
Gilroy, D. J., Kauffman, K. W., Hall, R. A., Huang, X. & Chu, F. S. Assessing potential health risks from microcystin toxins in blue-green algae dietary supplements. Environ. Health Perspect. 108,435439 (2000).
Giovannoni, S. J., E. F. DeLong, T. M. Schmidt, and N. R. Pace. 1990. Tangential flow filtration and preliminary phylogenetic analysis of marine picoplankton. Appl. Environ. Microbiol. 56:2572-2575.
Goldberg, J., Huang, H. B., Kwon, Y. G., Greengard, P., Nairn, A-C., Kuriyan, J., 1995. Three-dimensional structure of the catalytic subunit of protein serine/threonine phosphatase-1. Nature 376, 745-53.
Grüntzig, V., S. C. Nold, J. Zhou, and J. M. Tiedje. 2001. Pseudomonas stutzeri nitrite reductase gene abundance in environmental samples measured by real-time PCR. Appl. Environ. Microbiol. 67:760-768.
Gugger, M., C. Lyra, P. Henriksen, A. Couté, J.-F. Humbert, and K Sivonen. 2002. Phylogenetic comparison of the cyanobacterial genera Anabaena and Aphanizomenon. Int. J. Syst. Evol. Microbiol. 52:1867-1880.
Hall, T. A. BioEdit: a user-friendly biological sequence alignment editor and analysis program for Windows 95/98/NT. Nucl. Acids. Symp. Ser. 41, 95-98 (1999).
Harada, K.-I., F. Kondo, and L. Lawton. 1999. Laboratory analysis of cyanotoxins. p. 369-405. In I. Chorus and J. Bartram (ed.), Toxic cyanobacteria in water. E & FN Spon. London and New York.
Heckman, D. S. et al. Molecular evidence for the early colonization of land by fungi and plants. Science 293, 1129-1133 (2001).
Heid, C. A., J. Stevens, K J. Livak, and P. M. Williams. 1996. Real time quantitative PCR. Genome Res. 6:986-994.
Herdman, M., M. Janvier, R. Rippka, and R. Y. Stanier. 1979. Genome size of cyanobacteria. J. Gen. Microbiol. 111:73-85.
Hisbergues, M., Christiansen, G., Rouhiainen, L., Sivonen, K & Börner, T. PCR-based identification of microcystin producing genotypes of different cyanobacterial genera: application to environmental samples. Arch Microbiol. 180, 402-410 (2003).
Hopwood, D. A. Genetic contributions to understanding polyketide synthases. Chem. Rev. 97, 2465-2497 (1997).
Ikeda, H., Nonomiya, T., Usami, M., Ohta, T., Omura, S., 1999. Organization of the biosynthetic gene cluster for the polyketide anthelmintic macrolide avermectin in Streptomyces avermitilis. Proc. Natl. Acad. Sci. USA 96, 9509-9514.
Kaebernick, M., T. Rohrlack, K Chiistoffersen, and B. A. Neilan. 2001. A spontaneous mutant of microcystin biosynthesis: genetic characterization and effect on Daphnia. Environ. Microbiol. 3:669-679.
Kagan, R. M., Clarke, S., 1994. Widespread occurrence of three sequence motifs in diverse S-adenosylmethionine-dependent methyltransferases suggests a common structure for these enzymes. Arch. Biochem. Biophys. 310, 417-427.
Kohli, R. M., Trauger, J. W., Schwarzer, D., Marahiel, M. A., Walsh, C. T., 2001. Generality of peptide cyclization catalyzed by isolated thioesterase domains of nonribosomal peptide. Biochemistry 40, 7099-7108.
Kotai, J., 1972. Instructions for preparation of modified nutrient solution Z8 for algae, publication B-11/69, Norwegian Institute for Water Research. Blindern, Oslo.
Kuiper-Goodman, T., Falconer, I. & Fitzgerald, J. in Toxic Cyanobacteria in Water. A Guide to their Public Health Consequences, Monitoring and Management (eds Chorus, I. & Bartham, J.) 113-153 (E and FN Spoon, London, 1999).
Kurmayer, R., E. Dittmann, J. Fastner, and I. Chorus. 2002. Diversity of microcystin genes within a population of the toxic cyanobacterium Microcystis spp. in Lake Wannsee (Berlin, Germany). Microb. Ecol. 43:107-118.
Labarre, J., F. Chauvat, and P. Thuriaux. 1989; Insertional mutagenesis by random cloning of antibiotic resistance genes into the genome of the cyanobacterium Synechocystis strain PCC 6803. J. Bacteriol. 171:3449-3457.
Lahti, K, J. Rapala, M. Färdig, M. Niemelä, and K Sivonen. 1997. Persistence of cyanobacterial hepatotoxin, microcystin-LR in particulate material and dissolved in lake water. Wat. Res. 31:1005-1012.
Lee, S. J., M.-H. Jang, H.-S. Kim, B.-D. Yoon, and H.-M. Oh. 2000. Variation of microcystin content of Microcystis aeruginosa relative to medium N:P ratio and growth stage. J. Appl. Microbiol. 89:323-329.
Lepere, C., Wilmotte, A. & Meyer B. Molecular diversity of Microcystis strains (Cyanophyceae, Chroococcales) based on 16S rDNA sequences. Syst. Geogr. Pl. 70, 275-283 (2000).
Lindroos K et al, 2002 Nucleic Acid Research vol 30, no 14e70.
Lyra, C., Suomalainen, S., Gugger, M., Vezie, C., Sundman, P., Paulin, L., Sivonen, K, 2001. Molecular characterization of planktic cyanobacteria of Anabaena, Aphanizomenon, Microcystis and Planktothrix genera Int. J. System. Evol. Microbiol. 51, 513-526.
MacKintosh, C., Beattie, K. A., Klumpp, S., Cohen, P., Codd, G. A., 1990. Cyanobacterial microcystin-LR is a potent and specific inhibitor of protein phosphatase 1 and 2A from both mammals and higher plants. FEBS Lett. 264, 187-192;
Marahiel, M. A., Stachelhaus, T., Mootz, H. D., 1997. Modular peptide synthetases involved in nonribosomal peptide synthesis. Chem. Rev. 97, 2651-2673.
Mehta, P. K, Hale, T. I., Christen, P., 1993. Aminotranferases: demonstration of homology and division into evolutionary subgroups. Eur. J. Biochem. 214, 549-561.
Moffitt, M. C. & Neilan, B. A. On the presence of peptide synthetase and polyketide synthase genes in the cyanobacterial genus Nodularia. FEMS Microbiol. Lett. 196, 207-214 (2001).
Moon-van der Staay, S-Y., Wachter, R. & Vaulot, D. Oceanic 18S rDNA sequences from picoplankton reveal unsuspected eukaryotic diversity. Nature 409, 607-610 (2001).
Moore, R. E., Chen, J. L, Moore, B. S., Patterson, G. M. L., Charmichael, W. W., 1991. Biosynthesis of microcystin-LR. Origin of the carbons in the Adda and Masp units. J. Am Chem. Soc. 113, 5083-5084.
Namikoshi, M., K L. Rinehart, R. Sakai, R. R. Stotts, A. M. Dahlem, V. R. Beasley, W. W. Carmichael, and W. R. Evans. 1992. Identification of 12 hepatotoxins from a Horner Lake bloom of the cyanobacteria Microcystis aeruginosa, Microcystis viridis, and Microcystis wesenbergii: nine new microcystins. J. Org. Chem. 57:866-872.
Namikoshi, M., Rinehart, KL., 1996. Bioactive combounds produced by cyanobacteria. J. Ind. Microbiol. 17, 373-384.
Neilan, B. A, D. Jacobs, T. del Dot, L. L. Blackall, P. R. Hawkins, P. T. Cox, and A. E. Goodman. 1997. rRNA sequences and evolutionary relationships among toxic and nontoxic cyanobacteria of the genus Microcystis. hit. J. Syst. Bacteriol. 47:693-697.
Neilan, B. A. et al. Nonribosomal peptide synthesis and toxigenity of cyanobacteria. J. Bacteriol. 181, 4089-4097 (1999).
Nishizawa, T., M. Asayama, K. Fujii, K−1. Harada, and M. Shirai. 1999. Genetic analysis of the peptide synthetase genes for a cyclic heptapeptide microcystin in Microcystis spp. J. Biochem. 126:520-529.
Nishizawa, T., Ueda, A., Asayama, M., Fujii, K., Harada, K-, Ochi, K, Shirai, M., 2000. Polyketide synthase gene coupled to the peptide synthetase module involved in the biosynthesis of the cyclic heptapeptide microcystin. J. Biochem. 127, 779-789.
Nishizawa, T., Asayama, M., Shirai, M., 2001. Cyclic heptapeptide microcystin biosynthesis requires the glutamate racemase gene. Microbiology 147, 1235-1241.
Normeman, D., and P. V. Zimba. 2002. A PCR-based test to assess the potential for microcystin occurrence in channel catfish production ponds. J. Phycol. 38:230-233.
Ohtake, A., M. Shirai, T. Aida, N. Mori, K.-I. Harada, KI Matsuura, M. Suzuki, and M. Nakano. 1989. Toxicity of Microcystis species isolated from natural blooms and purification of the toxin. Appl. Environ. Microbiol. 55:3202-3207.
Orr, P. T., and G. J. Jones. 1998. Relationship between microcystin production and cell division rates in nitrogen-limited Microcystis aeruginosa cultures. Limnol. Oceanogr. 43:1604-1614.
Otsuka, S. et al. Phylogenetic relationships between toxic and non-toxic strains of the genus Microcystis based on 16S to 23S internal transcribed spacer sequence. FEMS Microbiol. Lett. 172, 15-21 (1999).
Pan, H., L. Song, Y. Liu, and T. Börner. 2002. Detection of hepatotoxic Microcystis strains by PCR with intact cells from both culture and environmental samples. Arch. Microbiol. 178:421-427.
Rapala, J., K Sivonen, C. Lyra, and S. L Niemeli. 1997. Variation of microcystins, cyanobacterial hepatotoxins in Anabaena spp. as a function of growth stimuli. Appl. Environ. Microbiol. 63:2206-2212.
Rapala, J., and K Sivonen. 1998. Assessment of environmental conditions that favor hepatotoxic and neurotoxic Anabaena spp. strains cultured under light limitation at different temperatures. Microb. Ecol. 36:181-192.
Repka, S., J. Mehtonen, J. Vaitomaa, L. Saari, and K. Sivonen. 2001. Effects of nutrients on growth and nodularin production of Nodularia strain GR8b. Microb. Ecol. 42:606-613.
Ressom, R. et al. Health Effects of Toxic Cyanobacteria (Blue-green Algae) (Australian Government Publishing Service, Canberra, 1994).
Reynolds, C. S. 1997. Vegetation processes in the pelagic: a model for ecosystem theory. Excellence in Ecology, Ecology Institute, Oldendorf/Luhe, 371 p.
Rippka, R., and M. Herdman. 1992. Pasteur culture collection of cyanobacterial strains in axenic culture. Catalogue & taxonomic handbook, volume I: catalogue of strains 1992/1993. Paris, Institut Pasteur. p. 103.
Rippka, R., Castenholz, R. W., Iteman, I., Herdman, M., 2001. Form-genus I. Anabaena Bory de St. Vincent 1822 sensu Rippka, Demelles, Waterbury, Herdman and Stanier 1979. In: Boone, D. R., Castenholz, R. W. (Eds.), Bergey's Manual of Systematic Bacteriology. Springer-Verlag, New York, pp. 566-568. 2nd edn, vol. 1.
Rouhiainen, L., Sivonen, K, Buikema, W. J., Haselkorn, R., 1995. Characterization of toxin-producing cyanobacteria by using an oligonucleotide probe containing a tandemly repeated heptamer. J. Bacteriol. 177, 6021-6026.
Rouhiainen, L., Paulin, L., Suomalainen S., Hyytiäinen, H., Buikema, W., Haselkorn, R., Sivonen, K, 2000. Genes encoding synthetases of cyclic depsipeptides, anabaenopeptilides, in Anabaena strain 90. Mol. Microbiol. 37, 156-167.
Saitou N., Nei M. The neighbor-joining method: a new method for reconstructing phylogenetic trees. Mol Biol Evol. 4, 406-25 (1987).
Sambrook, J., Fritsch, E. F., Maniatis, T., 1989. Molecular cloning: A Laboratory Manual. Cold Spring Harbor Laboratory Press, Plainview, N.Y., 2nd edn.
Scrutton, N. S., Beny, A., Perham, R. N., 1990. Redesign of the coenzyme specificity of a dehydrogenase by protein engineering. Nature 343, 38-43.
Silakowski, B., Nordsiek, G., Kunze, B., Blocker, H., Müller R., 2001. Novel features in a combined polyketide synthase/non-ribosomal peptide synthetase: the myxalamid biosynthetic gene cluster of the myxobacterium Stigmatella aurantiaca Sga 15. Chem. Biol. 8, 59-69.
Sivonen, K., K Himberg, R. Luukkainen, S. L. Niemelä, G. K Poon, and G. A. Codd. 1989. Preliminary characterization of neurotoxic cyanobacteria blooms and strains from Finland Toxic. Assess. 4:339-352.
Sivonen, K, W. W. Carmichael M. Namikoshi, K. L. Rinehart, A. M. Dahlem, and S. I. Niemelä. 1990. Isolation and characterization of hepatotoxic microcystin homologs from the filamentous freshwater cyanobacterium Nostoc sp. strain 152. Appl. Environ. Microbiol. 56:2650-2657.
Sivonen, K, Namikoshi, M., Evans, W. R., Carmichael W. W., Sun, F., Rouhiainen, L., Luukkainen, R., Rinehart, K. L., 1992. Isolation and characterization of a variety of microcystins from seven strains of the cyanobacterial genus Anabaena. Appl. Environ. Microbiol. 58, 2495-2500.
Sivonen, K, M. Namikoshi, R. Luukkainen, M. Fardig, L. Rouhiainen, W. R. Evans, W. W. Carmichael, K L. Rinehart, and S. I. Niemela. 1995. Variation of cyanobacterial hepatotoxins in Finland, p. 163-169. In M. Munavar and M. Luotola (ed.), The contaminants in the nordig ecosystem: dynamics, processes & fate. Ecovision world monograph series, Asssterdam, the Neatherlands.
Sivonen, K, Jones, G., 1999. Cyanobacterial toxins. In: Chorus, I., Bertram J. (Eds.), Toxic Cyanobacteria in Water: A Guide to their Public Health Consequences, Monitoring and Management. E. & F. N. Spon, London, pp. 41-111.
Stachelhaus, T., Mootz, H. D., Marahiel M. A., 1999. The specificity-conferring code of adenylation domains in normbosomal peptide synthetases. Chem. Biol. 6, 493-505.
Suzuki, M. T., and S. J. Giovannoni. 1996. Bias caused by template annealing in the amplification of mixtures of 16S rRNA genes by PCR. Appl. Environ. Microbiol. 62:625-630.
Swofford, D. L., Olsen, G. J., Waddell, P. J. & Hillis, D. M. in Molecular Systematics (eds Hillis, D. M., Moritz, C. & Mable, B. K) 407-514 (Sinauer, Sunderland, Mass., 1996).
Swofford, D. L. PAUP*. Phylogenetic Analysis Using Parsimony (*and Other Methods) Version 4.0b8 (Sinaeur, Sunderland, Mass., 2001).
Tang, L., Yoon, Y. J., Choi, C-Y., Hutchinson, C. R., 1998. Characterization of the enzymatic domains in the modular polyketide synthase involved in rifamycin B biosynthesis by Amycolatopsis mediterranei. Gene 216, 255-265.
Taton, A., S. Grubisic, E. Brambilla, R. De Wit and A. Wilmotte (2003). Cyanobacterial Diversity in Natural and Artificial Microbial Mats of Lake Fryxell (McMurdo Dry Valleys, Antarctica): a Morphological and Molecular Approach. Appl. Environ. Microbiol. 69, 5157-5169.
Thompson J D, Higgins D G, Gibson T J. CLUSTAL W: improving the sensitivity of progressive multiple sequence alignment through sequence weighting, position-specific gap penalties and weight matrix choice. Nucleic Acids Res 22, 4673-80 (1994).
Tillett, D., Dittmann, E., Erhard, M., von Döhren, H., Borner, T., Neilan, B. A., 2000. Structural organization of microcystin biosynthesis in Microcystis aeruginosa PCC7806: an integrated peptide-polyketide synthetase system. Chem. Biol. 7, 753-764.
Tillett, D., Parker, D. L. & Neilan, B. A. Detection of toxigenicity by a probe for the microcystin synthetase A gene (mcyA) of the cyanobacterial genus Microcystis: comparison of toxicities with 16S rRNA and phycocyanin operon (phycocyanin intergenic spacer) phylogenies. Appl. Environ. Microbiol. 67, 2810-2818 (2001).
Tsuge, K, Akiyama, T., Shoda, M., 2001. Cloning, sequencing, and characterization of the iturin A operon. J. Bacteriol. 183, 6265-6273.
Utermöhl, H. 1958. Zur Vervollkommnung der quantitativen phytoplankton-methodik. Mitt. Int. Ver. Limnol. 9:1-38.
Utkilen, H. & Gjøhme, N. Iron-stimulated toxin production in Microcystis aeruginosa. Appl. Environ. Microbiol. 61, 797-800 (1995).
Vasconcelos, V. M., K Sivonen, W. R. Evans, W. W. Carmichael, and M. Namikoshi. 1995. Isolation and characterization of microcystins (haptapeptide hepatotoxins) from Portuguese strains of Microcystis aeruginosa KUTZ. emend ELEKIN. Arch. Hydrobiol. 134:295-305.
Vezie, C., L. Brient, K. Sivonen, G. Bertru, J.-C. Lefeuvre, and M. Salkinoja-Salonen. 1998. Variation of microcystin content of cyanobacterial blooms and isolated strains in Lake Grand-Lieu (France). Microb. Ecol. 35:126-135.

Vézie, C., J. Rapala, J. Vaitomaa, J. Seitsonen, and K. Sivonen. 2002. Effect of nitrogen and phosphorus on growth of toxic and nontoxic Microcystis strains and on intracellular microcystin concentrations. Microb. Ecol. 43:443-454.
Wawrik, B., J. H. Paul, and F. R. Tabita. 2002. Real-time PCR quantification of rbcL (ribulose-1,5-bisphosphate carboxylase/oxygenase) mRNA in diatoms and pelagophytes. Appl. Environ. Microbiol. 68:3771-3779.
Willén, T. 1962. Studies on the phytoplankton of some lakes connected with or recently isolated from the Baltic. Oikos 13:169-199.
Wintzingerode F., U. B. Gobel, and E. Stackebrandt. 1997. Determination of microbial diversity in environmental samples: pitfalls of PCR-based rRNA analysis. FEMS Microbiol. Rev. 21:213-229.

1. A method for detecting toxic cyanobacteria, characterized in that the method comprises that nucleic acid from a biological sample is brought into contact with an oligonucleotide designed to be specific for the mcyE gene, and the presence or absence of toxic cyanobacteria is detected by a suitable molecular biology method.

2. The method according to claim 1, wherein the oligonucleotide is designed to be specific for a region of the mcyE gene responsible for adding Adda and D-glutamate to the immature synthesis product of microcystin.

3. The method according to claim 1, wherein the oligonucleotide is designed to be specific for a region of the mcyE gene comprising two domains, the adenylation domain and the domain which catalyses a peptide bond between Adda-D-glutamate dipeptide and dehydroalanine.

4. The method according to claim 1, wherein the oligonucleotide is designed to be specific for a fragment of the mcyE gene selected from the group of genera Anabaena, Microcystis, Planktothrix, Nostoc and Nodularia.

5. The method according to claim 1, wherein the nucleic acid from a biological sample is DNA or RNA.

6. The method according to claim 1, wherein the oligonucleotide is designed to be specific for a fragment of the mcyE gene selected from the group of sequences SEQ ID NO. 1 to SEQ ID NO: 34 as shown in FIG. 19 A to H or to a fragment of said sequences.

7. The method according to claim 1, wherein the oligonucleotide is designed to be specific for a fragment of the mcyE gene selected from the group of consensus sequences SEQ ID NO: 35 to SEQ ID NO: 39 as shown in FIG. 15 A to C or to a fragment of said sequences.

8. The method according to claim 1, wherein the oligonucleotide is selected from the group of mcyE-F2 (SEQ ID NO: 64), AnamcyE-12R (SEQ ID NO: 65) and MicmcyE-R8 (SEQ ID NO:66).

9. The method according to claim 1, wherein the oligonucleotide is selected from the group of discriminating probes SEQ ID NO: 40 to SEQ ID NO: 45.

10. The method according to claim 1, wherein the oligonucleotide is selected from the group of common probes SEQ ID NO: 46 to SEQ ID NO: 51.

11. The method according to claim 1, wherein the detection is combined with a detection method using oligonucleotides designed to be specific for any other mcy gene, such as mcyA or mcyD, or for 16S rRNA.

12. The method according to claim 1, wherein detection is combined with a detection method selected from the group of measuring microcystin concentration, determining cell number, cell density or determining biomass.

13. A fragment of the mcyE gene, characterized in that it is on the region of the mcyE gene responsible for adding Adda and D-glutamate to the immature synthesis product of microcystin and that it is or is located in any of the sequences selected from the group comprising SEQ ID NO. 1 to SEQ ID NO: 34 as shown in FIG. 19 A to H, or is a sequence having at least 80% identity, preferably 90% identity to the sequence.

14. A fragment of the mcyE gene, characterized in that it is on the region of the mcyE gene responsible for adding Adda and D-glutamate to the immature synthesis product of microcystin and that it is or is located in any of the sequences selected from the group comprising consensus sequences SEQ ID NO: 35 to SEQ ID NO: 39 as shown in FIG. 15 A to C, or is a sequence having at least 80% identity, preferably 90% identity to the sequence.

15. An oligonucleotide, characterized in that it is designed to be specific for the region of the mcyE gene responsible for adding Adda and D-glutamate to the immature synthesis product of microcystin that is or is located in any of the sequences selected from the group comprising SEQ ID NO: 1 to SEQ ID NO: 34 as shown in FIG. 19 A to H or selected from the group comprising any of the consensus sequences SEQ ID NO: 35 to SEQ ID NO: 39 as shown in FIG. 15 A to C.

16. An oligonucleotide selected from the group of mcyE-F2 (SEQ ID NO: 64), AnamcyE-12R (SEQ ID NO: 65) and MicmcyE-R8 (SEQ ID NO:66).

17. An oligonucleotide selected from the group of discriminating probes of SEQ ID NO: to SEQ ID NO: 45.

18. An oligonucleotide selected from the group of common probes of SEQ ID NO: 46 to SEQ ID NO: 51.

19. mcyE gene from Anabaena genus encoding the amino acid sequence of SEQ ID NO: or a sequence having at least 80% identity, preferably 90% identity to the sequence, or a fragment of said sequence having polymorphic sites which make possible of designing oligonucleotides to be specific for the fragment.

20. mcyE gene from Anabaena genus having the nucleic acid sequence SEQ ID NO: 68 or a sequence having at least 80% identity, preferably 90% identity to the sequence, or a fragment of said sequence having polymorphic sites, which make possible of designing oligonucleotides to be specific for the fragment.

21. The method according to claim 12, wherein the detection is combined with a detection method using oligonucleotides designed to be specific for a fragment of the mcyD gene which is on the region of the mcyD gene responsible for chain elongation of the growing Adda amino acid in the synthesis of microcystin and that it is or is located in any of the sequences selected from the group comprising sequences SEQ ID NO: 131 to SEQ ID NO: 149 as shown in FIG. 38 A to F or is a sequence having at least 85% identity, preferably 90% identity to the sequence.

22. The method according to claim 12, wherein the detection is combined with a detection method using oligonucleotides designed to be specific for mcyD gene from Anabaena genus encoding the amino acid sequence of SEQ ID NO: 69 or a sequence having at least 80% identity, preferably 90% identity to the sequence, or a fragment of said sequence having polymorphic sites which make possible of designing oligonucleotides to be specific for the fragment.

23. The method according to claim 12, wherein the detection is combined with a detection method using oligonucleotides designed to be specific for mcyD gene from Anabaena genus having the nucleic acid sequence SEQ ID NO: 70 or a sequence having at least 80% identity, preferably 90% identity to the sequence or a fragment of said sequence having polymorphic sites which make possible of designing oligonucleotides to be specific for the fragment.

24. An oligonucleotide selected from the group of discriminating probes of SEQ ID NO:71 to SEQ ID NO:90.

25. An oligonucleotide selected from the group of common probes of SEQ ID NO:91 to SEQ ID NO:110.

26. An oligonucleotide selected from the group of discriminating probes of SEQ ID NO:150 to SEQ ID NO:163.

27. An oligonucleotide selected from the group of common probes of SEQ ID NO:157 to SEQ ID NO:163.

28. A kit for detection of toxic cyanobacteria by microarray method, characterized in that it comprises
discriminating probes and common probes designed to be specific for mcy-E gene, and optionally for mcyD gene;
DNA or RNA zip and complementary zip codes assigned to be specific for selected cyanobacteria genera.

29. A kit for detection of toxic cyanobacteria by hybridization, characterized in that it comprises
primers designed to be specific for the mcyE gene, and optionally for mcyD gene;
probes designed to be specific for selected cyanobacteria genera.

30. The kit according to claim 29, which comprises in addition to primers and probes designed to be specific for mcy gene also primers and probes for 16S rRNA gene.

31. A method for detecting toxic and non-toxic cyanobacteria, characterized in that the method comprises that nucleic acid from a biological sample is brought into contact with an oligonucleotide designed to be specific for mcyE gene or for other mcy genes, such as mcyD gene, and with an oligonucleotide designed to be specific for the 16SrRNA gene, and the presence or absence of toxic cyanobacteria is detected by a suitable molecular biology method.

32. The method according to claim 31, wherein the oligonucleotides are designed to be specific for a region of the mcyE and for a region of 16SrRNA gene.

33. The method according to claim 1, wherein the molecular biology method is selected from the group comprising hybridization, PCR, reverse transcriptase PCR, QTR-PCR, LCR, LDR and minisequencing.

34. The method according to claim 1, wherein the detection is made by microarray method.

35. The kit according to claim 28, which comprises in addition to primers and probes designed to be specific for mcy gene also primers and probes for 16SrRNA gene.

36. The method according to claim 31, wherein the molecular biology method is selected form the group comprising hybridization, PCR, reverse transcriptase PCR, QTR-PCR, LCR, LDR and minisequencing.

37. The method according to claim 31, wherein the detection is made by microarray method.
